(19) United States
(12) Patent Application Publication
(10) Pub. No.: US 2019/0017117 A1
(43) Pub. Date: Jan. 17, 2019

(54) MARKERS OF STROKE AND STROKE SEVERITY

(71) Applicant: WEST VIRGINIA UNIVERSITY, Morgantown, WV (US)

(72) Inventors: Taura L. BARR, Waynesburg, PA (US); Richard GIERSCH, Morgantown, WV (US); Grant O'CONNELL, Morgantown, WV (US)

(21) Appl. No.: 15/743,610

(22) PCT Filed: Jul. 8, 2016
(86) PCT No.: PCT/US16/41585

§ 371 Date: Jan. 10, 2018

Related U.S. Application Data
(60) Provisional application No. 62/191,096, filed on Jul. 10, 2015.
(60) Provisional application No. 62/300,342, filed on Feb. 26, 2016.
(60) Provisional application No. 62/352,680, filed on Jun. 21, 2016.

(51) Int. Cl. C12Q 1/6883; G06F 19/20; G16H 50/20; C12Q 1/686

(52) U.S. Cl. CPC C12Q 1/6883; G06F 19/20; C12Q 2600/158; C12Q 1/686; G16H 50/20

(57) ABSTRACT

Provided herein are methods, kits, and devices for detecting ischemic stroke and identifying biomarkers of ischemic stroke. Evaluating the expression patterns of ischemic stroke biomarkers in biological samples can allow for the diagnosis of stroke in a time-sensitive and bedside manner.

### CROSS-REFERENCE

This application claims the benefit of U.S. Provisional Patent Application No. 62/191,096, filed on Jul. 10, 2015, and U.S. Provisional Patent Application No. 62/300,342, filed on Feb. 26, 2016, and U.S. Provisional Patent Application No. 62/352,680, filed on Jun. 21, 2016 which are herein incorporated by reference in their entireties.

### GOVERNMENT SUPPORT

This invention was made with the support of National Institute of Nursing Research (NINR) Grant Number HHSN263201100872P and Robert Wood Johnson Foundation Nurse Faculty Scholars Award #70319.

### BACKGROUND

Stroke is often defined as the interruption of blood flow to brain tissue. Specifically, strokes often occur when there is an interruption in blood flow by the blockage or rupture of a blood vessel that serves the brain. The administration of thrombolytic agents are an effective treatment for strokes, however, thrombolytic agents such as tissue plasminogen activator (tPA) must be administered within a finite period. Thus, early and rapid diagnosis of stroke is critical for treatment. In many cases, expert neurological assessment is often needed for accurate diagnosis of ischemic stroke. In institutions where advanced neuroimaging is available, CT or Mill is often used as a diagnostic and/or confirmatory tool. However, most health care institutions do not have access to advanced imaging technologies or the expertise required to make a confirmatory diagnosis of strokes. Ideally, it would be desirable to provide additional tools to diagnose strokes in a time sensitive manner. Evaluating the expression patterns of biomarkers in peripheral blood can allow for the diagnosis of stroke in a time-sensitive and bedside manner.

### BRIEF SUMMARY

Provided herein are methods, kits, and devices for assessing ischemic stroke in a subject.

In one aspect, disclosed herein are methods. In one aspect, the method can comprise measuring a level of cell-free nucleic acids in a sample from a subject. In one aspect, the method can further comprise comparing a level of cell-free nucleic acids to a reference level of cell-free nucleic acids in a reference sample. In one aspect, a reference sample can be from a stroke mimic subject. In one aspect, the method can further comprise determining whether a sample or a reference sample has a higher level of cell-free nucleic acids. In one aspect, the method can further comprise assessing ischemic stroke. In one aspect, assessing can differentiate an ischemic stroke from a stroke mimic. In one aspect, assessing can differentiate ischemic stroke from stroke mimic with a sensitivity of at least 80% and a specificity of at least 75%. In one aspect, determining that a sample has a higher level of cell-free nucleic acids as compared to a reference can be indicative of a subject being an ischemic stroke subject. In one aspect, at least one of the cell-free nucleic acids can comprise an epigenetic marker. In one aspect, an epigenetic marker can be specific to one or more types of cells. In one aspect, an epigenetic marker can be specific to a cell from a neurovascular unit. In one aspect, an epigenetic marker can comprise acetylation, methylation, ubiquitylation, phosphorylation, sumoylation, ribosylation, citrullination, or any combination thereof. In one aspect, assessing can differentiate ischemic stroke from stroke mimic with a sensitivity of at least 85%. In one aspect, assessing can differentiate ischemic stroke from stroke mimic with a specificity of at least 80%. In one aspect, measuring a level of cell-free nucleic acids in a sample can be performed by using a probe that binds to at least one of the cell-free nucleic acids in a sample. In one aspect, measuring a level of cell-free nucleic acids in a sample can be performed by polymerase chain reaction. In one aspect, the polymerase chain reaction can be real-time polymerase chain reaction. In one aspect, measuring a level of cell-free nucleic acids in a sample can be performed by determining a level of a gene or a fragment thereof in the sample. In one aspect, the gene can encode telomerase reverse transcriptase, beta-globin, cluster of differentiation 240D, a member of albumin family, ribonuclease P RNA component H1, Alu J element, endogenous retrovirus group 3, glyceraldehyde 3-phosphate dehydrogenase, N-acetylglucosamine kinase, or alcohol dehydrogenase. In one aspect, the gene can be telomerase reverse transcriptase. In one aspect, measuring a level of cell-free nucleic acids in a sample can comprises adding an exogenous polynucleotide to a sample. In one aspect, an exogenous polynucleotide can comprise a fragment of a gene encoding a green fluorescence protein. In one aspect, comparing a level of cell-free nucleic acids to a reference level of cell-free nucleic acids in a reference sample can be performed using an epigenetic marker detecting probe that binds to at least one of the subgroup of the cell-free nucleic acids. In one aspect, a probe can comprise a label. In one aspect, a label can comprise a fluorochrome or radioactive isotope. In one aspect, a probe can comprise a polynucleotide. In one aspect, a polynucleotide can hybridize with at least one of the cell-free nucleic acids in a sample. In one aspect, cell-free nucleic acids can comprise cell-free DNA. In one aspect, cell-free nucleic acids can comprise cell-free RNA. In one aspect, cell-free RNA can comprise mRNA. In one aspect, mRNA can be specific to one or more types of cells. In one aspect, cell-free RNA can comprise microRNA. In one aspect, microRNA can be specific to one or more types of cells. In one aspect, mRNA can be specific to a cell in a neurovascular unit. In one aspect, at least one of the cell-free nucleic acids can be derived from a neutrophil extracellular trap. In one aspect, a sample can comprise a body fluid. In one aspect, a body fluid can comprise urine. In one aspect, a body fluid can comprise blood or a fraction thereof. In one aspect, a body fluid can comprise a fraction of blood. In one aspect, a fraction of blood can be plasma. In one aspect, plasma can be isolated by centrifuging blood. In one aspect, a fraction of blood can be serum. In one aspect, a subject can exhibit an ischemic stroke symptom. In one aspect, a sample can be obtained from a subject within 12 hours from onset of an ischemic stroke symptom. In one aspect, a sample can be obtained from a subject within 4.5 hours from onset of an ischemic stroke symptom. In one aspect, assessing can comprise assessing stroke severity of a subject. In one aspect, assessing can comprise assessing activation of innate immune system. In one aspect, assessing activation of innate immune system can comprise determining a neutrophil count in a subject. In one aspect, a neutrophil count can be determined based on a level of cell-free nucleic acids in a sample. In one aspect, assessing can comprise assessing a stroke-induced injury in a subject. In one aspect, a stroke-induced injury can comprise a myocardial infarction. In one aspect, a stroke-induced injury can be assessed based on a level of cell-free nucleic acids in a sample. In one aspect, a stroke-induced injury can be as indicated by National Institutes of Health Stroke Scale. In one aspect, the method can further comprise assessing a level of cell-free nucleic acids derived from neutrophil extracellular traps. In one aspect, a method described herein can further comprise triaging a subject to a stroke-treatment facility based on the assessing. In one aspect, a method can further comprise administering a treatment to a subject. In one aspect, the administrating can be performed if a level of cell-free nucleic acids in a subject is higher than a reference level of cell-free nucleic acids and the administering may not be performed if a level of cell-free nucleic acids in a subject is equal to or less than a reference level of cell-free nucleic acids. In one aspect, a treatment can comprise a drug. In one aspect, a drug can be tissue plasminogen activator. In one aspect, a treatment can be administered within 4.5 hours of onset of an ischemic stroke symptom. In one aspect, a treatment can reduce a level of cell-free nucleic acids in a subject. In one aspect, a subject can be a mammal. In one aspect, a mammal can be a human. In one aspect, a reference level of cell-free nucleic acids can be stored in a database or on a server. In one aspect, the method further comprises determining a time of ischemic stroke symptom onset in a subject. In one aspect, a time of ischemic stroke symptom onset can be determined by correlating a level of cell-free nucleic acids in a sample with a time of ischemic stroke symptom onset. In one aspect, the method can further comprise assessing a risk of ischemic stroke in a subject. In one aspect, the method can further comprise detecting ischemic stroke in a subject when a level of cell-free nucleic acids in a subject is at least 1 fold higher as compared to a reference level of cell-free nucleic acids. In one aspect, an ischemic stroke can be detected in a subject when a level of cell-free nucleic acids in a subject is at least 3 fold higher as compared to a reference level of cell-free nucleic acids. In one aspect, ischemic stroke can be detected in a subject when a ratio of cell-free nucleic acids is higher as compared to a reference ratio. In one aspect, the method can further comprise measuring a profile of blood cells in a subject. In one aspect, a profile of blood cells can comprise white blood cell differentiation, levels of muscle-type creatine kinase and brain-type creatine kinase, a hematocrit percent, a prothrombin time, a white blood cell count, a lymphocyte count, a platelet count, a neutrophil percent in a sample, or a combination thereof. In one aspect, a method can be performed using a portable device. In one aspect, the method can further comprise repeating any one or more method described herein at different time points to monitor ischemic stroke in a subject. In one aspect, the method can further comprise repeating any one or more method described herein at different time points to monitor a subject. In one aspect, different time points can comprise 1 week, 2 weeks, 1 month, 2 months, 3 months, 6 months, 8 months, or 1 year. In one aspect, repeating any one or more method described herein can be performed following administration of a treatment to a subject. In one aspect, a level of cell-free nucleic acids in a sample can be determinative of a subject's response to a treatment. In one aspect, a response can be a favorable reaction to a treatment. In one aspect, a response can be an adverse reaction to a treatment. In one aspect, the level of cell-free nucleic acids in a sample can be determinative at least in part for whether a subject can be eligible for a clinical trial.

In one aspect, disclosed herein are methods. In one aspect, a method can comprise measuring a level of cell-free nucleic acids in a sample from a subject. In one aspect, the method can comprise comparing a level of cell-free nucleic acids to a reference level of cell-free nucleic acids in a reference sample. In one aspect, the reference sample can be from a non-ischemic stroke subject. In one aspect, the method can further comprise assessing ischemic stroke in a subject using a computer system. In one aspect, assessing can differentiate ischemic stroke from non-ischemic stroke with a sensitivity of at least 80% and a specificity of at least 75%. In one aspect, at least one of the cell-free nucleic acids can comprise an epigenetic marker. In one aspect, an epigenetic marker can be specific to one or more types of cells. In one aspect, an epigenetic marker can be specific to a cell from a neurovascular unit. In one aspect, an epigenetic marker can comprise acetylation, methylation, ubiquitylation, phosphorylation, sumoylation, ribosylation, citrullination, or any combination thereof. In one aspect, assessing can differentiate ischemic stroke from stroke mimic with a sensitivity of at least 85%. In one aspect, assessing can differentiate ischemic stroke from stroke mimic with a specificity of at least 80%. In one aspect, measuring a level of cell-free nucleic acids in a sample can be performed using a probe that binds to at least one of the cell-free nucleic acids in a sample. In one aspect, measuring a level of cell-free nucleic acids in a sample can be performed by polymerase chain reaction. In one aspect, the polymerase chain reaction can be real-time polymerase chain reaction. In one aspect, measuring a level of cell-free nucleic acids in a sample can be performed by determining a level of a gene or a fragment thereof in a sample. In one aspect, the gene can encode telomerase reverse transcriptase, beta-globin, cluster of differentiation 240D, a member of albumin family, ribonuclease P RNA component H1, Alu J element, endogenous retrovirus group 3, glyceraldehyde 3-phosphate dehydrogenase, N-acetylglucosamine kinase, or alcohol dehydrogenase. In one aspect, gene can be telomerase reverse transcriptase. In one aspect, measuring a level of cell-free nucleic acids in a sample can comprise adding an exogenous polynucleotide to a sample. In one aspect, an exogenous polynucleotide can comprise a fragment of a gene encoding a green fluorescence protein. In one aspect, comparing a level of cell-free nucleic acids to a reference level of cell-free nucleic acids in a reference sample can be performed using an epigenetic marker detecting probe that binds to at least one of the subgroup of the cell-free nucleic acids. In one aspect, a probe can comprise a label. In one aspect, a label can comprise a fluorochrome or radioactive isotope. In one aspect, a probe can comprise a polynucleotide. In one aspect, a polynucleotide can hybridize with at least one of the cell-free nucleic acids in a sample. In one aspect, cell-free nucleic acids can comprise cell-free DNA. In one aspect, cell-free nucleic acids can comprise cell-free RNA. In one aspect, cell-free RNA can comprise mRNA. In one aspect, mRNA can be specific to one or more types of cells. In one aspect, cell-free RNA can comprise microRNA. In one aspect, microRNA can be specific to one or more types of cells. In one aspect, mRNA can be specific to a cell in a neurovascular unit. In one aspect, at least one of the cell-free nucleic acids can be derived from a neutrophil extracellular trap. In one aspect, a sample can comprise a body fluid. In one aspect, a body fluid can comprise urine. In one aspect, a body fluid can comprise blood or a fraction thereof. In one aspect, a body fluid can comprise a fraction of blood. In one aspect, a fraction of blood can be plasma. In one aspect, plasma can be isolated by centrifuging blood. In one aspect, a fraction of blood can be serum. In one aspect, a subject can exhibit an ischemic stroke symptom. In one aspect, a sample can be obtained from a subject within 12 hours from onset of an ischemic stroke symptom. In one aspect, a sample can be obtained from a subject within 4.5 hours from onset of an ischemic stroke symptom. In one aspect, assessing can comprise assessing stroke severity of a subject. In one aspect, assessing can comprise assessing activation of innate immune system. In one aspect, assessing activation of innate immune system can comprise determining a neutrophil count in a subject. In one aspect, a neutrophil count can be determined based on a level of cell-free nucleic acids in a sample. In one aspect, assessing can comprise assessing a stroke-induced injury in a subject. In one aspect, a stroke-induced injury can comprise a myocardial infarction. In one aspect, a stroke-induced injury can be assessed based on a level of cell-free nucleic acids in a sample. In one aspect, a stroke-induced injury can be as indicated by National Institutes of Health Stroke Scale. In one aspect, the method can further comprise assessing a level of cell-free nucleic acids derived from neutrophil extracellular traps. In one aspect, the method can further comprise triaging a subject to a stroke-treatment facility based on the assessing. In one aspect, the method can further comprising administering a treatment to a subject. In one aspect, administrating can be performed if a level of cell-free nucleic acids in a subject is higher than a reference level of cell-free nucleic acids and administering may not be performed if a level of cell-free nucleic acids in a subject is equal to or less than a reference level of cell-free nucleic acids. In one aspect, a treatment can comprise a drug. In one aspect, a drug can be tissue plasminogen activator. In one aspect, a treatment can be administered within 4.5 hours of onset of an ischemic stroke symptom. In one aspect, a treatment can reduce a level of cell-free nucleic acids in a subject. In one aspect, a subject can be a mammal. In one aspect, a mammal can be a human. In one aspect, a reference level of cell-free nucleic acids can be stored in a database or on a server. In one aspect, the method can further comprise determining a time of ischemic stroke symptom onset in a subject. In one aspect, a time of ischemic stroke symptom onset can be determined by correlating a level of cell-free nucleic acids in a sample with a time of ischemic stroke symptom onset. In one aspect, the method can further comprise assessing a risk of ischemic stroke in a subject. In one aspect, the method can further comprise detecting ischemic stroke in a subject when a level of cell-free nucleic acids in a subject is at least 1 fold higher as compared to a reference level of cell-free nucleic acids. In one aspect, an ischemic stroke can be detected in a subject when a level of cell-free nucleic acids in a subject is at least 3 fold higher as compared to a reference level of cell-free nucleic acids. In one aspect, ischemic stroke can be detected in a subject when a ratio of cell-free nucleic acids is higher as compared to the reference ratio. In one aspect, the method can further comprise measuring a profile of blood cells in a subject. In one aspect, a profile of blood cells can comprise white blood cell differentiation, levels of muscle-type creatine kinase and brain-type creatine kinase, a hematocrit percent, a prothrombin time, a white blood cell count, a lymphocyte count, a platelet count, a neutrophil percent in a sample, or a combination thereof. In one aspect, a method can be performed using a portable device. In one aspect, the method can further comprise repeating any one or more method described herein at different time points to monitor ischemic stroke in a subject. In one aspect, the method can further comprise repeating any one or more method described herein at different time points to monitor a subject. In one aspect, different time points can comprise 1 week, 2 weeks, 1 month, 2 months, 3 months, 6 months, 8 months, or 1 year. In one aspect, repeating any one or more method described herein can be performed following administration of a treatment to a subject. In one aspect, a level of cell-free nucleic acids in a sample can be determinative of a subject's response to a treatment. In one aspect, a response can be a favorable reaction to a treatment. In one aspect, a response can be an adverse reaction to a treatment. In one aspect, the level of cell-free nucleic acids in a sample can be determinative at least in part for whether a subject can be eligible for a clinical trial.

In one aspect, disclosed herein are methods. In one aspect, a method can comprise measuring a level of cell-free nucleic acids carrying an epigenetic marker. In one aspect, cell-free nucleic acids are in a sample from a subject suspected of having an ischemic stroke. In one aspect, the method can further comprise comparing a level of cell-free nucleic acids to a reference level of cell-free nucleic acids carrying an epigenetic marker in a reference sample. In one aspect, a reference sample can be from a healthy control subject or a stroke mimic subject. In one aspect, the method can further comprise assessing ischemic stroke in a subject using a computer system, wherein assessing can differentiate ischemic stroke from a healthy control or a stroke mimic. In one aspect, assessing can differentiate ischemic stroke from a healthy control or a stroke mimic with a sensitivity of at least 80% and a specificity of at least 75%. In one aspect, at least one of the cell-free nucleic acids can comprise an epigenetic marker. In one aspect, an epigenetic marker can be specific to one or more types of cells. In one aspect, an epigenetic marker can be specific to a cell from a neurovascular unit. In one aspect, an epigenetic marker can comprise acetylation, methylation, ubiquitylation, phosphorylation, sumoylation, ribosylation, citrullination, or any combination thereof. In one aspect, assessing can differentiate ischemic stroke from stroke mimic with a sensitivity of at least 85%. In one aspect, assessing can differentiate ischemic stroke from stroke mimic with a specificity of at least 80%. In one aspect, measuring a level of cell-free nucleic acids in a sample can be performed using a probe that binds to at least one of the cell-free nucleic acids in a sample. In one aspect, measuring a level of cell-free nucleic acids in a sample can be performed by polymerase chain reaction. In one aspect, the polymerase chain reaction can be real-time polymerase chain reaction. In one aspect, measuring a level of cell-free nucleic acids in a sample can be performed by determining a level of a gene or a fragment thereof in a sample. In one aspect, the gene can encode telomerase reverse transcriptase, beta-globin, cluster of differentiation 240D, a member of albumin family, ribonuclease P RNA component H1, Alu J element, endogenous retrovirus group 3, glyceraldehyde 3-phosphate dehydrogenase, N-acetylglucosamine kinase, or alcohol dehydrogenase. In one aspect, gene can be telomerase reverse transcriptase. In one aspect, measuring a level of cell-free nucleic acids in a sample can comprises adding an exogenous polynucleotide to a sample. In one aspect, an exogenous polynucleotide can comprise a fragment of a gene encoding a green fluorescence protein. In one aspect, comparing a level of cell-free nucleic acids to a reference level of cell-free nucleic acids in a reference sample can be performed using an epigenetic marker detecting probe that binds to at least one of the subgroup of the cell-free nucleic acids. In one aspect, a probe can comprise a label. In one aspect, a label can comprise a fluorochrome or radioactive isotope. In one aspect, a probe can comprise a polynucleotide. In one aspect, a polynucleotide can hybridize with at least one of the cell-free nucleic acids in a sample. In one aspect, cell-free nucleic acids can comprise cell-free DNA. In one aspect, cell-free nucleic acids can comprise cell-free RNA. In one aspect, cell-free RNA can comprise mRNA. In one aspect, mRNA can be specific to one or more types of cells. In one aspect, cell-free RNA can comprise microRNA. In one aspect, microRNA can be specific to one or more types of cells. In one aspect, mRNA can be specific to a cell in a neurovascular unit. In one aspect, at least one of the cell-free nucleic acids can be derived from a neutrophil extracellular trap. In one aspect, a sample can comprise a body fluid. In one aspect, a body fluid can comprise urine. In one aspect, a body fluid can comprise blood or a fraction thereof. In one aspect, a body fluid can comprise a fraction of blood. In one aspect, a fraction of blood can be plasma. In one aspect, plasma can be isolated by centrifuging blood. In one aspect, a fraction of blood can be serum. In one aspect, a subject can exhibit an ischemic stroke symptom. In one aspect, a sample can be obtained from a subject within 12 hours from onset of an ischemic stroke symptom. In one aspect, a sample can be obtained from a subject within 4.5 hours from onset of an ischemic stroke symptom. In one aspect, assessing can comprise assessing stroke severity of the subject. In one aspect, assessing can comprise assessing activation of innate immune system. In one aspect, assessing activation of innate immune system can comprise determining a neutrophil count in a subject. In one aspect, a neutrophil count can be determined based on a level of cell-free nucleic acids in a sample. In one aspect, assessing can comprise assessing a stroke-induced injury in a subject. In one aspect, a stroke-induced injury can comprise a myocardial infarction. In one aspect, a stroke-induced injury can be assessed based on a level of cell-free nucleic acids in a sample. In one aspect, a stroke-induced injury can be as indicated by National Institutes of Health Stroke Scale. In one aspect, the method can further comprise assessing a level of cell-free nucleic acids derived from neutrophil extracellular traps. In one aspect, the method can further comprise triaging a subject to a stroke-treatment facility based on the assessing. In one aspect, the method can further comprise administering a treatment to a subject. In one aspect, the administrating can be performed if a level of cell-free nucleic acids in a subject is higher than a reference level of cell-free nucleic acids and administering may not be performed if a level of cell-free nucleic acids in a subject is equal to or less than a reference level of cell-free nucleic acids. In one aspect, treatment can comprise a drug. In one aspect, a drug can be tissue plasminogen activator. In one aspect, a treatment can be administered within 4.5 hours of onset of an ischemic stroke symptom. In one aspect, a treatment can reduce a level of cell-free nucleic acids in a subject. In one aspect, a subject can be a mammal. In one aspect, a mammal can be a human. In one aspect, a reference level of cell-free nucleic acids can be stored in a database or on a server. In one aspect, the method can further comprise determining a time of ischemic stroke symptom onset in a subject. In one aspect, a time of ischemic stroke symptom onset can be determined by correlating a level of cell-free nucleic acids in a sample with a time of ischemic stroke symptom onset. In one aspect, the method further comprises assessing a risk of ischemic stroke in a subject. In one aspect, the method can further comprise detecting ischemic stroke in a subject when a level of cell-free nucleic acids in a subject is at least 1 fold higher as compared to a reference level of cell-free nucleic acids. In one aspect, an ischemic stroke can be detected in a subject when a level of cell-free nucleic acids in a subject is at least 3 fold higher as compared to a reference level of cell-free nucleic acids. In one aspect, ischemic stroke can be detected in a subject when a ratio of cell-free nucleic acids is higher as compared to a reference ratio. In one aspect, the method can further comprise measuring a profile of blood cells in a subject. In one aspect, a profile of blood cells can comprise white blood cell differentiation, levels of muscle-type creatine kinase and brain-type creatine kinase, a hematocrit percent, a prothrombin time, a white blood cell count, a lymphocyte count, a platelet count, a neutrophil percent in a sample, or a combination thereof. In one aspect, a method can be performed using a portable device. In one aspect, the method can further comprise repeating any one or more method described herein at different time points to monitor ischemic stroke in a subject. In one aspect, the method can further comprise repeating any one or more method described herein at different time points to monitor a subject. In one aspect, different time points can comprise 1 week, 2 weeks, 1 month, 2 months, 3 months, 6 months, 8 months, or 1 year. In one aspect, repeating any one or more method described herein can be performed following administration of a treatment to a subject. In one aspect, a level of cell-free nucleic acids in a sample can be determinative of a subject's response to a treatment. In one aspect, a response can be a favorable reaction to a treatment. In one aspect, a response can be an adverse reaction to a treatment. In one aspect, a level of cell-free nucleic acids in a sample can be determinative at least in part for whether a subject can be eligible for a clinical trial.

In one aspect, disclosed herein are methods. In one aspect, the method can comprise measuring a level of cell-free nucleic acids in a sample from a subject suspected of having an ischemic stroke. The method can further comprise measuring a level of a subgroup of cell-free nucleic acids. In one aspect, the subgroup of cell-free nucleic acids can carry an epigenetic marker. In one aspect, the method can further comprise determining a ratio between a level of cell-free nucleic acids and a level of a subgroup of cell-free nucleic acids. The method can further comprise comparing a ratio between a level of cell-free nucleic acids and a level of a subgroup of cell-free nucleic acids to a reference ratio, wherein the reference ratio is a ratio between a level of cell-free nucleic acids in a reference sample and a level of a subgroup of cell-free nucleic acids in a reference sample, wherein the subgroup of cell-free nucleic acids in the reference sample carry an epigenetic marker. In one aspect, the reference sample can be from a healthy control subject or a stroke mimic subject. In one aspect, the method can further comprise assessing ischemic stroke in a subject using a computer system, wherein the assessing can differentiate ischemic stroke from a healthy control or a stroke mimic. In one aspect, assessing can differentiate ischemic stroke from a healthy control or a stroke mimic with a sensitivity of at least 80% and a specificity of at least 75%. In one aspect, at least one of the cell-free nucleic acids can comprise an epigenetic marker. In one aspect, an epigenetic marker can be specific to one or more types of cells. In one aspect, an epigenetic marker can be specific to a cell from a neurovascular unit. In one aspect, an epigenetic marker can comprise acetylation, methylation, ubiquitylation, phosphorylation, sumoylation, ribosylation, citrullination, or any combination thereof. In one aspect, assessing can differentiate ischemic stroke from stroke mimic with a sensitivity of at least 85%. In one aspect, assessing can differentiate ischemic stroke from stroke mimic with a specificity of at least 80%. In one aspect, measuring a level of cell-free nucleic acids in a sample can be performed using a probe that binds to at least one of the cell-free nucleic acids in a sample. In one aspect, measuring a level of cell-free nucleic acids in a sample can be performed by polymerase chain reaction. In one aspect, the polymerase chain reaction can be real-time polymerase chain reaction. In one aspect, measuring a level of cell-free nucleic acids in a sample can be performed by determining a level of a gene or a fragment thereof in a sample. In one aspect, the gene can encode telomerase reverse transcriptase, beta-globin, cluster of differentiation 240D, a member of albumin family, ribonuclease P RNA component H1, Alu J element, endogenous retrovirus group 3, glyceraldehyde 3-phosphate dehydrogenase, N-acetylglucosamine kinase, or alcohol dehydrogenase. In one aspect, gene a can be telomerase reverse transcriptase. In one aspect, measuring a level of cell-free nucleic acids in a sample can comprises adding an exogenous polynucleotide to a sample. In one aspect, an exogenous polynucleotide can comprise a fragment of a gene encoding a green fluorescence protein. In one aspect, comparing a level of cell-free nucleic acids to a reference level of cell-free nucleic acids in a reference sample can be performed using an epigenetic marker detecting probe that binds to at least one of the subgroup of the cell-free nucleic acids. In one aspect, a probe can comprise a label. In one aspect, a label can comprise a fluorochrome or radioactive isotope. In one aspect, a probe can comprise a polynucleotide. In one aspect, a polynucleotide can hybridize with at least one of the cell-free nucleic acids in a sample. In one aspect, cell-free nucleic acids can comprise cell-free DNA. In one aspect, cell-free nucleic acids can comprise cell-free RNA. In one aspect, cell-free RNA can comprise mRNA. In one aspect, mRNA can be specific to one or more types of cells. In one aspect, cell-free RNA can comprise microRNA. In one aspect, microRNA can be specific to one or more types of cells. In one aspect, mRNA can be specific to a cell in a neurovascular unit. In one aspect, at least one of the cell-free nucleic acids can be derived from a neutrophil extracellular trap. In one aspect, a sample can comprise a body fluid. In one aspect, a body fluid can comprise urine. In one aspect, a body fluid can comprise blood or a fraction thereof. In one aspect, a body fluid can comprise a fraction of blood. In one aspect, a fraction of blood can be plasma. In one aspect, plasma can be isolated by centrifuging blood. In one aspect, a fraction of blood can be serum. In one aspect, a subject can exhibit an ischemic stroke symptom. In one aspect, a sample can be obtained from a subject within 12 hours from onset of an ischemic stroke symptom. In one aspect, a sample can be obtained from a subject within 4.5 hours from onset of an ischemic stroke symptom. In one aspect, assessing can comprise assessing stroke severity of a subject. In one aspect, assessing can comprise assessing activation of innate immune system. In one aspect, assessing activation of innate immune system can comprise determining a neutrophil count in a subject. In one aspect, a neutrophil count can be determined based on a level of cell-free nucleic acids in a sample. In one aspect, assessing can comprise assessing a stroke-induced injury in a subject. In one aspect, a stroke-induced injury can comprise a myocardial infarction. In one aspect, a stroke-induced injury can be assessed based on a level of cell-free nucleic acids in a sample. In one aspect, a stroke-induced injury can be as indicated by National Institutes of Health Stroke Scale. In one aspect, the method can further comprise assessing a level of cell-free nucleic acids derived from neutrophil extracellular traps. In one aspect, the method can further comprise triaging a subject to a stroke-treatment facility based on the assessing. In one aspect, the method can further comprise administering a treatment to a subject. In one aspect, administrating can be performed if a level of cell-free nucleic acids in a subject is higher than a reference level of cell-free nucleic acids and administering may not be performed if a level of cell-free nucleic acids in a subject is equal to or less than a reference level of cell-free nucleic acids. In one aspect, a treatment can comprise a drug. In one aspect, a drug can be tissue plasminogen activator. In one aspect, a treatment can be administered within 4.5 hours of onset of an ischemic stroke symptom. In one aspect, a treatment can reduce a level of cell-free nucleic acids in a subject. In one aspect, a subject can be a mammal. In one aspect, a mammal can be a human. In one aspect, a reference level of cell-free nucleic acids can be stored in a database or on a server. In one aspect, the method can further comprise determining a time of ischemic stroke symptom onset in a subject. In one aspect, a time of ischemic stroke symptom onset can be determined by correlating a level of cell-free nucleic acids in a sample with a time of ischemic stroke symptom onset. In one aspect, the method can further comprise assessing a risk of ischemic stroke in the subject. In one aspect, the method can further comprise detecting ischemic stroke in a subject when a level of cell-free nucleic acids in a subject is at least 1 fold higher as compared to a reference level of cell-free nucleic acids. In one aspect, an ischemic stroke can be detected in a subject when a level of cell-free nucleic acids in a subject is at least 3 fold higher as compared to a reference level of cell-free nucleic acids. In one aspect, ischemic stroke can be detected in a subject when a ratio is higher as compared to a reference ratio. In one aspect, the method can further comprise measuring a profile of blood cells in a subject. In one aspect, a profile of blood cells can comprise white blood cell differentiation, levels of muscle-type creatine kinase and brain-type creatine kinase, a hematocrit percent, a prothrombin time, a white blood cell count, a lymphocyte count, a platelet count, a neutrophil percent in the sample, or a combination thereof. In one aspect, a method can be performed using a portable device. In one aspect, the method can further comprise repeating any one or more method described herein at different time points to monitor ischemic stroke in a subject. In one aspect, the method can further comprise repeating any one or more method described herein at different time points to monitor a subject. In one aspect, different time points can comprise 1 week, 2 weeks, 1 month, 2 months, 3 months, 6 months, 8 months, or 1 year. In one aspect, repeating any one or more method described herein can be performed following administration of a treatment to a subject. In one aspect, a level of cell-free nucleic acids in a sample can be determinative of a subject's response to a treatment. In one aspect, a response can be a favorable reaction to a treatment. In one aspect, a response can be an adverse reaction to a treatment. In one aspect, the level of cell-free nucleic acids in a sample can be determinative at least in part for whether a subject can be eligible for a clinical trial.

Disclosed herein are devices. In one aspect, a device can comprise a memory that stores executable instructions. In one aspect, the device can further comprise a processor that executes the executable instructions to perform the method of any one or more of the methods disclosed herein. In one aspect, the device can be a filament-based diagnostic device.

Disclosed herein are kits. In one aspect, a kit can comprise a probe for measuring a level of cell-free nucleic acids in a sample from the subject, wherein the probe binds to at least one of the cell-free nucleic acid in the sample. In one aspect, a kit can further comprise a detecting reagent to examining binding of the probe to at least one of the cell-free nucleic acids. In one aspect, a probe can be labeled. In one aspect, a probe can be labeled with a fluorochrome or radioactive isotope. In one aspect, a probe can be a polynucleotide.

Disclosed herein are kits. In one aspect, a kit can comprise a probe for measuring a level of cell-free nucleic acids carrying an epigenetic marker in a sample from a subject, wherein the probe binds to the cell-free nucleic acids carrying an epigenetic marker. In one aspect, a kit can further comprise a detecting reagent to examining binding of a probe with cell-free nucleic acids. In one aspect, a probe can be labeled. In one aspect, a probe can be labeled with a fluorochrome or radioactive isotope. In one aspect, a probe can be a polynucleotide.

In one aspect, disclosed herein is a method of assessing ischemic stroke in a subject suspected of having a condition. The method can comprise (a) measuring expression of a group of biomarkers comprising two or more biomarkers in a sample from the subject by contacting a panel of probes with the sample, wherein the probes bind to the group of biomarkers or molecules derived therefrom; (b) comparing the expression of the group of biomarkers in the sample to a reference, wherein the reference can comprise expression of the group of biomarkers in a healthy control subject and a stroke mimic subject; and (c) assessing ischemic stroke in the subject using a computer system, wherein the assessing can differentiate ischemic stroke from a healthy control and ischemic stroke from a stroke mimic with a sensitivity of at least 92% and a specificity of at least 92%. In some cases, probes can be labeled. In some cases, labeled probes can be labeled with a fluorochrome or radioactive isotope. In some cases, the group of biomarkers can comprise myelin and lymphocyte protein. In some cases, a group of biomarkers can comprise an inhibitor of Ras-ERK pathway. In some cases, the inhibitor of Ras-ERK pathway can be GRB2-related adaptor protein. In some cases, a group of biomarkers can comprise a member of inhibitor of DNA binding family. In some cases, the member of inhibitor of DNA binding family can be inhibitor of DNA binding 3. In some cases, a group of biomarkers can comprise a lysosomal cysteine proteinase. In some cases, the lysosomal cysteine proteinase can be cathepsin. In some cases, the cathepsin can be cathepsin Z. In some cases, a group of biomarkers can comprise a motor protein. In some cases, the motor protein can be a kinesin-like protein. In some cases, the kinesin-like protein can be kinesin-like protein 1B. In some cases, a group of biomarker can comprise a receptor for pigment epithelium-derived factor. In some cases, the receptor for pigment epithelium-derived factor can be a plexin domain-containing protein. In some cases, the plexin domain-containing protein can be plexin domain-containing protein 2. In some cases, the methods can further comprise detecting ischemic stroke in a subject. In some cases, the methods can further comprise detecting ischemic stroke in a subject when expression of at least one of anthrax toxin receptor 2, serine/threonine-protein kinase 3, pyruvate dehydrogenase lipoamide kinase isozyme 4, and cluster of differentiation 163 is increased compared to a reference. In some embodiments, an increase can be by at least 1 fold compared to a reference. In some cases, the methods can further comprise detecting ischemic stroke in a subject when, expression of at least one of myelin and lymphocyte protein, GRB2-related adaptor protein, and inhibitor of DNA binding 3 is decreased. In some cases, the decrease can be by at least 1 fold compared to a reference. In some cases, the methods can further comprise detecting ischemic stroke in a subject when expression of at least one of anthrax toxin receptor 2, serine/threonine-protein kinase 3, pyruvate dehydrogenase lipoamide kinase isozyme 4, and cluster of differentiation 163 is increased and expression of at least one of myelin and lymphocyte protein, GRB2-related adaptor protein, and inhibitor of DNA binding 3 is decreased compared to a reference. In some cases, the expression of a group of biomarkers can be measured using an immunoassay, polymerase chain reaction, or a combination thereof. In some cases, the expression of a group of biomarkers can be measured by polymerase chain reaction. In some cases, the polymerase chain reaction can be quantitative reverse transcription polymerase chain reaction. In some cases, a reference can be stored in a database or on a server. In some cases, expression of a group of biomarker in a sample from a subject can comprise RNA expression, protein expression, or a combination thereof.

In another aspect, disclosed herein is a method of assessing ischemic stroke in a subject suspected of having a condition. The method can comprise: (a) measuring expression of a group of biomarkers comprising two or more biomarkers in a sample from the subject by contacting a panel of probes with the sample, wherein the probes bind to the group of biomarkers or molecules derived therefrom; (b) comparing the expression of the group of biomarkers in the sample to a reference, wherein the reference can be expression of the group of biomarkers in a non-ischemic stroke subject; and (c) assessing ischemic stroke in the subject using a computer system, wherein the assessing can have a sensitivity of at least 92% and a specificity of at least 92% based on expression of two biomarkers in the group of biomarkers. In some cases, probes can be labeled. In some cases, labeled probes can be labeled with a fluorochrome or radioactive isotope. In some cases, a group of biomarkers can comprise myelin and lymphocyte protein. In some cases, a group of biomarkers can comprise an inhibitor of Ras-ERK pathway. In some cases, the inhibitor of Ras-ERK pathway can be GRB2-related adaptor protein. In some cases, a group of biomarkers comprises a member of inhibitor of DNA binding family. In some cases, the member of inhibitor of DNA binding family can be inhibitor of DNA binding 3. In some cases, a group of biomarkers can comprise a lysosomal cysteine proteinase. In some cases, the lysosomal cysteine proteinase can be cathepsin. In some cases, the cathepsin can be cathepsin Z. In some cases, a group of biomarkers can comprise a motor protein. In some cases, the motor protein can be a kinesin-like protein. In some cases, the kinesin-like protein can be kinesin-like protein 1B. In some cases, a group of biomarker can comprise a receptor for pigment epithelium-derived factor. In some cases, the receptor for pigment epithelium-derived factor can be a plexin domain-containing protein. In some cases, the plexin domain-containing protein can be plexin domain-containing protein 2. In some cases, the methods can further comprise detecting ischemic stroke in a subject. In some cases, the methods can further comprise detecting ischemic stroke in a subject when expression of at least one of anthrax toxin receptor 2, serine/threonine-protein kinase 3, pyruvate dehydrogenase lipoamide kinase isozyme 4, and cluster of differentiation 163 is increased compared to a reference. In some embodiments, the increase can be by at least 1 fold compared to the reference. In some cases, the methods can further comprise detecting ischemic stroke in a subject when, expression of at least one of myelin and lymphocyte protein, GRB2-related adaptor protein, and inhibitor of DNA binding 3 is decreased. In some cases, the decrease can be by at least 1 fold compared to a reference. In some cases, the methods can further comprise detecting ischemic stroke in a subject when expression of at least one of anthrax toxin receptor 2, serine/threonine-protein kinase 3, pyruvate dehydrogenase lipoamide kinase isozyme 4, and cluster of differentiation 163 is increased and expression of at least one of myelin and lymphocyte protein, GRB2-related adaptor protein, and inhibitor of DNA binding 3 is decreased compared to a reference. In some cases, the expression of a group of biomarkers can be measured using an immunoassay, polymerase chain reaction, or a combination thereof. In some cases, the expression of a group of biomarkers can be measured by polymerase chain reaction. In some cases, the polymerase chain reaction can be quantitative reverse transcription polymerase chain reaction. In some cases, a reference can be stored in a database or on a server. In some cases, expression of a group of biomarker in a sample from a subject can comprise RNA expression, protein expression, or a combination thereof.

In another aspect, disclosed herein is a method of assessing ischemic stroke in a subject suspected of having a condition. The method can comprise: (a) measuring expression of a group of biomarkers in a sample from the subject by contacting a panel of labeled probes with the sample, wherein the labeled probes bind to the group of biomarkers or molecules derived therefrom, and wherein the group of biomarkers can comprise two or more of (i) an anthrax toxin receptor, (ii) a serine/threonine-protein kinase, (iii) a pyruvate dehydrogenase lipoamide kinase, and (iv) a cluster of differentiation family member; (b) comparing the expression of the group of biomarkers in the sample to a reference, wherein the reference can be expression of the group of biomarkers in a non-ischemic stroke subject; and (c) assessing ischemic stroke in the subject using a computer system. In some cases, a group of biomarkers can comprise an anthrax toxin receptor. In some cases, the anthrax toxin receptor can be anthrax toxin receptor 2. In some cases, a group of biomarkers can comprise a serine/threonine-protein kinase. In some cases, the serine/threonine-protein kinase can be serine/threonine-protein kinase 3. In some cases, a group of biomarkers can comprise a pyruvate dehydrogenase lipoamide kinase. In some cases, the pyruvate dehydrogenase lipoamide kinase can be pyruvate dehydrogenase lipoamide kinase isozyme 4. In some cases, a group of biomarkers can comprise a cluster of differentiation family member. In some cases, the cluster of differentiation family member can be cluster of differentiation 163. In some cases, the methods can further comprise detecting ischemic stroke in a subject when expression of at least one biomarker in a group of biomarkers is increased compared to the reference. In some embodiments, the increase can be by at least 1 fold compared to the reference. In some cases, a group of biomarkers can further comprise one or more of: (i) myelin and lymphocyte protein, (ii) an inhibitor of Ras-ERK pathway, (iii) a member of inhibitor of DNA binding family, (iv) a lysosomal cysteine proteinase, (v) a motor protein, and (vi) a receptor for pigment epithelium-derived factor. In some cases, a group of biomarkers can comprise myelin and lymphocyte protein. In some cases, a group of biomarkers can comprise an inhibitor of Ras-ERK pathway. In some cases, the inhibitor of Ras-ERK pathway can be GRB2-related adaptor protein. In some cases, a group of biomarkers can comprise a member of inhibitor of DNA binding family. In some cases, the member of inhibitor of DNA binding family can be inhibitor of DNA binding 3. In some cases, a group of biomarkers can comprise a lysosomal cysteine proteinase. In some cases, the lysosomal cysteine proteinase can be cathepsin. In some cases, the cathepsin can be cathepsin Z. In some cases, a group of biomarkers can comprise a motor protein. In some cases, the motor protein can be a kinesin-like protein. In some cases, the kinesin-like protein can be kinesin-like protein 1B. In some cases, a group of biomarker can comprise a receptor for pigment epithelium-derived factor. In some cases, the receptor for pigment epithelium-derived factor can be a plexin domain-containing protein. In some cases, the plexin domain-containing protein can be plexin domain-containing protein 2. In some cases, the methods can further comprise detecting ischemic stroke in a subject. In some cases, the methods can further comprise detecting ischemic stroke in a subject when expression of at least one of anthrax toxin receptor 2, serine/threonine-protein kinase 3, pyruvate dehydrogenase lipoamide kinase isozyme 4, and cluster of differentiation 163 is increased compared to a reference. In some embodiments, the increase can be by at least 1 fold compared to the reference. In some cases, the methods can further comprise detecting ischemic stroke in a subject when, expression of at least one of myelin and lymphocyte protein, GRB2-related adaptor protein, and inhibitor of DNA binding 3 is decreased. In some cases, the decrease can be by at least 1 fold compared to a reference. In some cases, the methods can further comprise detecting ischemic stroke in a subject when expression of at least one of anthrax toxin receptor 2, serine/threonine-protein kinase 3, pyruvate dehydrogenase lipoamide kinase isozyme 4, and cluster of differentiation 163 is increased and expression of at least one of myelin and lymphocyte protein, GRB2-related adaptor protein, and inhibitor of DNA binding 3 is decreased compared to a reference. In some cases, the expression of a group of biomarkers can be measured using an immunoassay, polymerase chain reaction, or a combination thereof. In some cases, the expression of a group of biomarkers can be measured by polymerase chain reaction. In some cases, the polymerase chain reaction can be quantitative reverse transcription polymerase chain reaction. In some cases, a reference can be stored in a database or on a server. In some cases, expression of a group of biomarker in a sample from a subject can comprise RNA expression, protein expression, or a combination thereof.

In another aspect, disclosed herein is a method of assessing ischemic stroke in a subject suspected of having a disease or condition. The method can comprise: (a) measuring expression of a group of biomarkers in a sample from the subject by contacting a panel of labeled probes with the sample, wherein the labeled probes bind to the group of biomarkers or molecules derived therefrom, and wherein the group of biomarkers comprises two or more of anthrax toxin receptor 2, serine/threonine-protein kinase 3, pyruvate dehydrogenase lipoamide kinase isozyme 4, and cluster of differentiation 163; (b) comparing the expression of the group of biomarkers to a reference, wherein the reference can be the expression of the group of biomarkers in a non-ischemic stroke subject; and (c) assessing ischemic stroke in the subject using a computer system, whereby the expression of the two or more biomarkers in the sample in an amount that is greater than expression of the two or more biomarkers in the reference can be indicative of ischemic stroke. In some cases, a group of biomarkers can further comprise one or more of myelin and lymphocyte protein, GRB2-related adaptor protein, inhibitor of DNA binding 3, cathepsin Z, kinesin-like protein 1B, and plexin domain-containing protein 2. In some cases, labeled probes can be labeled with a fluorochrome or radioactive isotope. In some cases, the group of biomarkers can comprise a first subgroup of biomarkers comprising anthrax toxin receptor 2, serine/threonine-protein kinase 3, pyruvate dehydrogenase lipoamide kinase isozyme 4, and cluster of differentiation 163, and a second subgroup of biomarkers comprising one or more of myelin and lymphocyte protein, GRB2-related adaptor protein, and inhibitor of DNA binding 3. In some cases, ischemic stroke can be detected in a subject when expression of a first subgroup of biomarkers is increased by at least 1 fold and expression of a second subgroup of biomarkers is decreased by at least 1 fold compared to a reference. In some cases, a first subgroup of biomarkers further comprises one or more of cathepsin Z, kinesin-like protein 1B, and plexin domain-containing protein 2. In some cases, a group of biomarkers can comprise a first subgroup of biomarkers comprising anthrax toxin receptor 2, serine/threonine-protein kinase 3, pyruvate dehydrogenase lipoamide kinase isozyme 4, cluster of differentiation 163, cathepsin Z, kinesin-like protein 1B, and plexin domain-containing protein 2. In some cases a second subgroup of biomarkers can comprise myelin and lymphocyte protein, GRB2-related adaptor protein, and inhibitor of DNA binding 3. In some aspects, ischemic stroke can be detected in a subject when expression of a first subgroup of biomarkers is increased by at least 1 fold and expression of a second subgroup of biomarkers is decreased by at least 1 fold compared to a reference. In some cases, ischemic stroke in a subject can be detected with a sensitivity of at least 90% and a specificity of at least 90%. In some cases, a group of biomarkers can comprise anthrax toxin receptor 2, serine/threonine-protein kinase 3, pyruvate dehydrogenase lipoamide kinase isozyme 4, and cluster of differentiation 163, and wherein ischemic stroke in a subject can be detected with a sensitivity of at least 98% and a specificity of at least 98%. In some cases, the expression of a group of biomarkers can be measured using an immunoassay, polymerase chain reaction, or a combination thereof. In some cases, the expression of a group of biomarkers can be measured by polymerase chain reaction. In some cases, a polymerase chain reaction can be quantitative reverse transcription polymerase chain reaction. In some cases, probes can be contacted with a sample within 24 hours from ischemic stroke symptom onset in a subject. In some cases, probes can comprise polynucleotides. In some cases, polynucleotides can hybridize with mRNA of a group of biomarkers. In some cases, polynucleotides can hybridize with DNA derived from mRNA of a group of biomarkers. In some cases, probes can comprise polypeptides. In some cases, polypeptides can bind to proteins of a group of the biomarkers. In some cases, polypeptides can be antibodies or fragments thereof. In some cases, a non-ischemic stroke subject can have a transient ischemic attack, a non-ischemic stroke, or a stroke mimic. In some cases, a non-ischemic stroke can be a hemorrhagic stroke. In some cases, a reference can be stored in a database or on a server. In some cases, expression of a group of biomarker in a sample from a subject can comprise RNA expression, protein expression, or a combination thereof. In some cases, expression of a group of biomarker can be a predictive indicator of a future ischemic stroke. In some cases, expression of a group of biomarker can be an indicator of an ischemic stroke severity. In some cases, methods can further comprise determining a time of ischemic stroke symptom onset in a subject. In some cases, a time of ischemic stroke symptom onset can be determined by correlating the expression of the group of biomarkers in a sample with the time of ischemic stroke symptom onset. In some cases, ischemic stroke can be detected within 24 hours from ischemic stroke symptom onset. In some cases, ischemic stroke can be detected within 4.5 hours from ischemic stroke symptom onset. In some cases, methods can further comprise administering a drug for treating ischemic stroke in a subject if ischemic stroke is detected. In some cases, a drug can be tissue plasminogen activator. In some cases, a drug reduces or inhibits expression or function of one or more biomarkers in a group of biomarkers in the subject. In some cases, a drug increases expression or function of one or more biomarkers in a group of biomarkers in a subject. In some cases, a drug can be administered within 4.5 hours from ischemic stroke symptom onset. In some cases, a subject can be a human. In some cases, a sample can be blood or a fraction of blood. In some cases, a fraction of blood can be plasma or serum. In some cases, methods can further comprise measuring a profile of blood cells in a subject. In some cases, a profile of blood cells can comprise white blood cell differentiation, levels of muscle-type creatine kinase and brain-type creatine kinase, a hematocrit percent, a prothrombin time, a white blood cell count, a lymphocyte count, a platelet count, a neutrophil percent in the sample, or a combination thereof. In some cases, measuring and assessing can be performed using a portable device. In some cases, assessing ischemic stroke in a subject can comprise assessing a risk of ischemic stroke in a subject. In some cases, a disease or condition can be ischemic stroke. In some cases, a disease or condition can be a stroke mimic. In some cases, there can be a likelihood of ischemic stroke in the subject if the expression of one or more biomarkers in a group of biomarkers is increased compared to a reference. In some cases, there can be a likelihood of ischemic stroke in the subject if the expression of one or more biomarkers in a group of biomarkers is decreased compared to the reference. In some cases, a likelihood of ischemic stroke can be indicated by a second assessment. In some cases, detection of ischemic stroke can be indicated by a second assessment. In some cases, a second assessment can be performed using a neuroimaging technique. In some cases, a neuroimaging technique can be computerized tomography scan, magnetic resonance imaging, or a combination thereof. In some cases, methods can further comprise repeatedly measuring expression of a group of biomarkers in a sample from, comparing the expression of a group of biomarkers to a reference, and assessing ischemic stroke at different time points to monitor ischemic stroke. In some cases, different time points can be within 1 week, 2 weeks, 1 month, 2 months, 3 months, 6 months, 8 months, or 1 year. In some cases, repeating measuring expression of a group of biomarkers in a sample from, comparing the expression of a group of biomarkers to a reference, and assessing ischemic stroke can be performed following administration of a treatment to a subject. In some cases, the expression of a group of biomarkers can be determinative of a subject's response to a treatment. In some cases, response can be an adverse reaction. In some cases, response can be a beneficial reaction to treatment. In some cases, expression of a group of biomarkers can be determinative at least in part for whether the subject is eligible for a clinical trial.

In another aspect, disclosed herein is a method of assessing ischemic stroke in a subject suspected of having ischemic stroke. The method can comprise: (a) measuring expression of a group of biomarkers in a sample from the subject by contacting a panel of probes with the sample, wherein the probes bind to a group of biomarkers or molecules derived therefrom, and wherein the group of biomarkers comprises two or more of anthrax toxin receptor 2, serine/threonine-protein kinase 3, pyruvate dehydrogenase lipoamide kinase isozyme 4, and cluster of differentiation 163; (b) comparing the expression of the group of biomarkers to a reference; and (c) assessing ischemic stroke in the subject using a computer system, wherein the assessing has a sensitivity of at least 90% and a specificity of at least 90%.

In another aspect, disclosed herein is a method of assessing ischemic stroke in a subject suspected of having ischemic stroke, the method comprising: (a) measuring expression of a group of biomarkers in a sample using an assay selected from the group consisting of an immunoassay, a polymerase chain reaction, and a combination thereof, wherein the group of biomarkers comprises two or more of anthrax toxin receptor 2, serine/threonine-protein kinase 3, pyruvate dehydrogenase lipoamide kinase isozyme 4, and cluster of differentiation 163; (b) comparing the expression of the group of biomarkers to a reference; and (c) assessing ischemic stroke in the subject using a computer system.

In another aspect, disclosed herein is a method of assessing ischemic stroke in a subject suspected of having ischemic stroke, the method comprising: (a) measuring expression of a group of biomarkers in a sample from the subject by contacting a panel of probes with the sample, wherein the probes bind to a group of biomarkers or molecules derived therefrom, and wherein the group of biomarkers comprises two or more of anthrax toxin receptor 2, serine/threonine-protein kinase 3, pyruvate dehydrogenase lipoamide kinase isozyme 4, and cluster of differentiation 163; (b) comparing the expression of the group of biomarkers to a reference; and (c) assessing ischemic stroke in the subject using a computer system, wherein ischemic stroke is detected in the subject if expression of at least one biomarker in the group of biomarkers is increased by at least 1 fold.

In another aspect, disclosed herein is a method of assessing ischemic stroke in a subject suspected of having ischemic stroke, the method comprising: (a) measuring expression of a group of biomarkers in a sample from the subject using an assay selected from the group consisting of an immunoassay, a polymerase chain reaction, and a combination thereof, wherein the assay can be performed by contacting a panel of labeled probes with the sample, wherein the labeled probes bind to a group of biomarkers or molecules derived therefrom, and wherein the group of biomarkers comprises two or more of anthrax toxin receptor 2, serine/threonine-protein kinase 3, pyruvate dehydrogenase lipoamide kinase isozyme 4, and cluster of differentiation 163; (b) comparing the expression of the group of biomarkers to a reference; and (c) assessing ischemic stroke in the subject using a computer system, wherein ischemic stroke is detected in the subject if expression of at least one biomarkers in the group of biomarkers is increased by at least 1 fold, and wherein the assessing has a sensitivity of at least 90% and a specificity of at least 90%.

In another aspect, disclosed herein is a method of predicting a response of a subject suspected of having ischemic stroke to a treatment, the method comprising: (a) measuring expression of a group of biomarkers in a sample from the subject by contacting a panel of labeled probes with the sample, wherein the labeled probes bind to a group of biomarkers or molecules derived therefrom, and wherein the group of biomarkers comprises two or more of anthrax toxin receptor 2, serine/threonine-protein kinase 3, pyruvate dehydrogenase lipoamide kinase isozyme 4, and cluster of differentiation 163; (b) comparing the expression of the group of biomarkers to a reference; (c) administering the treatment to the subject; and (d) predicting the response of the subject to the treatment.

In another aspect, disclosed herein is a method of evaluating a drug, the method comprising: (a) measuring expression of a group of biomarkers in a sample from the subject by contacting a panel of labeled probes with the sample, wherein the labeled probes bind to a group of biomarkers or molecules derived therefrom, and wherein the group of biomarkers comprises two or more of anthrax toxin receptor 2, serine/threonine-protein kinase 3, pyruvate dehydrogenase lipoamide kinase isozyme 4, and cluster of differentiation 163; (b) administering the drug to the subject; (c) contacting the probes to a second sample, wherein the second sample can be obtained from the subject after the subject is administered the drug; (d) comparing the expression of the group of biomarkers in the first sample and the expression of the group of biomarkers in the second sample; and (e) evaluating the drug by analyzing difference between the expression of the group of biomarkers in the first sample and the expression of the group of biomarkers in the second sample.

In another aspect, disclosed herein is a kit for assessing ischemic stroke in a subject suspected of having ischemic stroke, the kit comprising: (a) a panel of probes for measuring expression of a group of biomarkers comprising two or more biomarkers, wherein the probes bind to the group of biomarkers or molecules derived therefrom; and (b) a detecting reagent for examining binding of the probes with the group of biomarkers, wherein the kit assesses ischemic stroke with a sensitivity of at least 92% and a specificity of at least 92% based on expression of two biomarkers in the group of biomarkers.

In another aspect, disclosed herein is a kit for assessing ischemic stroke in a subject suspected of having ischemic stroke, the kit comprising a panel of probes for measuring expression of a group of biomarkers comprising two or more of: (i) an anthrax toxin receptor, (ii) a serine/threonine-protein kinase, (iii) a pyruvate dehydrogenase lipoamide kinase, and (iv) a cluster of differentiation, wherein the probes bind to the group of biomarkers or molecules derived therefrom; and (b) a detecting reagent for examining binding of the probes with the group of biomarkers. In some cases, the group of biomarkers further comprises: myelin and lymphocyte protein, an inhibitor of Ras-ERK pathway, a member of inhibitor of DNA binding family, a lysosomal cysteine proteinase, a motor protein, and a receptor for pigment epithelium-derived factor.

In another aspect, disclosed herein is a kit for assessing ischemic stroke in a subject suspected of having ischemic stroke, the kit comprising: (a) a panel of probes for measuring expression of a group of biomarkers comprising two or more of anthrax toxin receptor 2, serine/threonine-protein kinase 3, pyruvate dehydrogenase lipoamide kinase isozyme 4, cluster of differentiation 163, wherein the probes bind to the group of biomarkers or molecules derived therefrom; and (b) a detecting reagent for examining binding of the probes with the group of biomarkers. In some cases, the group of biomarkers can further comprise myelin and lymphocyte protein, GRB2-related adaptor protein, inhibitor of DNA binding 3, cathepsin Z, kinesin-like protein 1B, and plexin domain-containing protein 2. In some cases, the panel of probes can comprise polynucleotides. In some cases, the polynucleotides can hybridize with mRNA of the group of biomarkers. In some cases, the polynucleotides can hybridize with DNA derived from mRNA of the group of biomarkers. In some cases, the panel of probes cam comprise polypeptides. In some cases, the polypeptides can bind to proteins of the group of biomarkers. In some cases, the polypeptides can be antibodies or fragments thereof. In some cases, at least one probe in the panel of probes can be labeled. In some cases, at least one probe in the panel of probes can be labeled with a fluorochrome or radioactive isotope. In some cases, a detecting reagent can bind to the panel of probes. In some cases, a detecting reagent can comprise a fluorescent or radioactive label. In some cases, the kits can further comprise a computer-readable medium for analyzing difference between the expression of the group of biomarkers and a reference.

In one aspect, provided herein is a method of detecting ischemic stroke in a subject, the method comprising: a) measuring a profile of a first group of biomarkers of ischemic stroke in a first sample from the subject, wherein the first group of biomarkers comprises a first class of biomolecules and the first class of biomolecules comprises at least one of a polynucleotide, a polypeptide, a carbohydrate, adaptamer or a lipid; b) measuring a profile of a second group of biomarkers of ischemic stroke in a second sample from the subject, wherein the second group of biomarkers comprises a second class of biomolecules, wherein the second class of biomolecules can be different from the first class of biomolecules and the second class of biomolecules comprises at least one of a polynucleotide, a polypeptide, a carbohydrate, adaptamer or a lipid; c) analyzing the profile of the first group of biomarkers of ischemic stroke and the profile of the second group of biomarkers of ischemic stroke with a computer system; and d) detecting ischemic stroke in the subject. In some cases, a first class of biomolecules can comprise a polynucleotide. In some cases, a second class of biomolecules can comprise a polypeptide. In some cases, a first class of biomolecules can comprise an adaptamer. In some cases, a second class of biomolecules can comprise an adaptamer. In some cases, a first class of biomolecules can comprise a polynucleotide and a second class of biomolecules can comprise a polypeptide. In some cases, a first class of biomolecules can comprise polynucleotides encoding one or more cytokines, and/or wherein a second class of biomolecules can comprise the one or more cytokines. In some cases, a profile of the second group of biomarkers of ischemic stroke can be measured by mass spectrometry, a multiplex assay, a microarray, an enzyme-linked immunosorbent assay, or any combination thereof. In some cases, analyzing can comprise comparing a profile of a first group of biomarkers of ischemic stroke to a reference profile. In some cases, analyzing can comprise comparing a profile of a second group of biomarkers of ischemic stroke to a reference profile. In some cases, detecting can comprise identifying a pattern of expression in a profile of a first group of biomarkers of ischemic stroke, and/or a profile of a second group of biomarkers of ischemic stroke. In some cases, detecting can comprise identifying a pattern of expression in a profile of a first group of biomarkers of ischemic stroke, and/or a profile of a second group of biomarkers of ischemic stroke.

In another aspect, disclosed herein is a method of detecting ischemic stroke in a subject, the method comprising: a) measuring a profile of biomarkers of ischemic stroke in a first sample from the subject; b) measuring a profile of blood cells in a second sample from the subject; c) analyzing the profile of biomarkers of ischemic stroke and the profile of blood cells with a computer system; and d) detecting ischemic stroke in the subject. In some cases, biomarkers of ischemic stroke can be polynucleotides. In some cases, biomarkers of ischemic stroke can be polypeptides. In some cases, analyzing can comprise comparing a profile of biomarkers of ischemic stroke to a reference profile. In some cases, measuring a profile of blood cells can comprise measuring at least one of CK-MB, a hematocrit percent, a prothrombin time, a white blood cell count, a lymphocyte count, a platelet count or a neutrophil percent. In some cases, measuring a profile of blood cells can comprise measuring white blood cell differential in a second sample. In some cases, analyzing can comprise comparing white blood cell differential to a white blood cell differential reference profile. In some cases, detecting can comprise identifying a pattern of expression in a profile of biomarkers of ischemic stroke, and/or a profile of blood cells. In some cases, a pattern of expression can be indicative of an ischemic stroke in a subject. In some cases, a pattern of expression can be a ratio of biomarker expression. In some cases, a pattern of expression can be the relative expression level of one or more biomarkers in disease and non-disease samples. In some cases, a profile of biomarkers of ischemic stroke can be measured by mass spectrometry, a multiplex assay, a microarray, an enzyme-linked immunosorbent assay, or any combination thereof. In some cases, a subject can be a human. In some cases, detecting can comprise assessing a presence or absence of a stroke mimic in a subject. In some cases, the methods disclosed herein can predict an outcome of ischemic stroke in a subject. In some cases, the methods disclosed herein can determine a time of ischemic stroke symptom onset in a subject. In some cases, the time of ischemic stroke symptom onset can be determined by correlating a profile of biomarkers with a time of ischemic stroke symptom onset. In some cases, ischemic stroke can be detected within 24 hours from ischemic stroke onset. In some cases, ischemic stroke can be detected within 4.5 hours from ischemic stroke onset. In some cases, methods provided herein can further comprise administering tissue plasminogen activator to a subject.

In one aspect, further disclosed herein is a method of identifying one or more biomarkers of ischemic stroke, the method comprising: a) measuring a profile of polynucleotides in a first ischemic stroke sample; b) measuring a profile of polypeptides in a second ischemic stroke sample; c) analyzing the profile of polynucleotides and the profile of polypeptides; and d) identifying the one or more biomarkers of ischemic stroke. In some cases, analyzing can comprise comparing the profile of polynucleotides to a polynucleotide reference profile, thereby identifying a first group of biomarkers in a first ischemic stroke sample. In some cases, a polynucleotide can be identified as one of a first group of biomarkers when an expression level difference in a polynucleotide of at least 1.5 fold is detected in a first ischemic stroke sample when compared to a polynucleotide reference profile. In some cases, analyzing can comprise comparing a profile of polypeptides to a polypeptide reference profile, thereby identifying a second group of biomarkers in a second ischemic stroke sample. In some cases, a polypeptide can be identified as one of a second group of biomarkers when an expression level difference in a polypeptide of at least 1.5 fold is detected in a second ischemic stroke sample when compared to a polypeptide reference profile. In some cases, identifying one or more biomarkers can comprise analyzing a first group of biomarkers and a second group of biomarkers. In some cases, analyzing a first group of biomarkers and second group of biomarkers can comprise identifying a polynucleotide of a first group of biomarkers as one of the one or more biomarkers of ischemic stroke when a polynucleotide encodes a polypeptide of a second group of biomarkers. In some cases, analyzing a first group of biomarkers and a second group of biomarkers can comprise identifying a polypeptide of a second group of biomarkers as one of the one or more biomarkers of ischemic stroke when a polypeptide is encoded by a polynucleotide of a first group of biomarkers. In some cases, profile of polypeptides can be measured by mass spectrometry, a multiplex assay, a microarray, an enzyme-linked immunosorbent assay, or any combination thereof. In some cases, polynucleotides can comprise polynucleotides encoding one or more of Chemokine (C-C motif) ligand 19 (CCL19), Chemokine (C-C motif) ligand 21 (CCL21), Galectin 3, Receptor for advanced glycation end-products (RAGE), Epithelial neutrophil-activating protein 78 (ENA78), Granulocyte-macrophage colony-stimulating factor (GM-CSF), Cluster of differentiation 30 (CD30), chemokine receptor 7 (CCR7), chondroitin sulfate proteoglycan 2 (CSPG2), IQ motif-containing GTPase activation protein 1 (IQGAP1), orosomucoid 1 (ORM1), arginase 1 (ARG1), lymphocyte antigen 96 (LY96), matrix metalloproteinase 9 (MMP9), carbonic anhydrase 4 (CA4), s100 calcium binding proteinA12 (s100A12), toll-like receptor 2 (TLR2), toll-like receptor 4 (TLR4), myeloid differentiation primary response gene 88 (MYD88), Janus Kinase 2 (JAK2), cluster of differentiation 3 (CD3), cluster of differentiation 4 (CD4), spleen tyrosine kinase (SYK), A kinase anchor protein 7 (AKAP7), CCAAT/enhancer binding protein (CEBPB), interleukin 10 (IL10), interleukin 8 (IL8), interleukin 22 receptor (IL22R) or an active fragment thereof. In some cases, polypeptides can comprise at least one of CCL19, CCL21, Galectin 3, RAGE, ENA78, GMCSF, CD30, CCR7, CSPG2, IQGAP1, ORM1, ARG1, LY96, MMP9, CA4, s100A12, Nav3, SAA, IGα, IGγ, IGκ, IGλ, or an active fragment thereof. In some cases, polypeptides can comprise one or more cytokines. In some cases, one or more cytokines can comprise BAFF, MMP9, APP, Aggrecan, Galectin 3, Fas, RAGE, Ephrin A2, CD30, TNR1, CD27, CD40, TNFα, IL6, IL8, IL10, IL1β, IFNγ, RANTES, IL1α, IL4, IL17, IL2, GMCSF, ENA78, IL5, IL12P70, TARC, GroAlpha, IL33, BLCBCA, IL31, MCP2, IGG3, IGG4, Isoform 2 of Teneurin 1, and isoform 2 of a Disintegrin or any active fragment thereof. In some cases, a reference profile can be obtained from a non-ischemic stroke subject. In some cases, a non-ischemic stroke subject can have a transient ischemic attack, a non-ischemic stroke, or a stroke mimic. In some cases, a non-ischemic stroke can be a hemorrhagic stroke. In some cases, polynucleotides can be RNA or DNA. In some cases, RNA can be mRNA. In some cases, DNA can be cell-free DNA. In some cases, DNA can be genomic DNA. In some cases, a first sample and/or a second can be blood or a fraction of blood. In some cases, a first ischemic stroke sample and/or a second ischemic stroke sample can be blood or a fraction of blood. In some cases, blood can be peripheral blood. In some cases, a fraction of blood can be plasma or serum. In some cases, a fraction of blood can comprise blood cells.

In another aspect, provided herein also includes a kit for detecting ischemic stroke in a subject, the kit comprising: a) a first panel of probes for detecting at least one of a first group of biomarkers of ischemic stroke, wherein the first group of biomarkers comprises a first class of biomolecules; and b) a second panel of probes for detecting at least one of a second group of biomarkers of ischemic stroke, wherein the second group of biomarkers comprises a second class of biomolecules. In some cases, a first panel of probes can be oligonucleotides capable of hybridizing to at least one of a first group of biomarkers of ischemic stroke. In some cases, a first class of biomolecules can be polynucleotides. In some cases, a first class of biomolecules can be aptamers. In some cases, polynucleotides can comprise polynucleotides encoding one or more of Chemokine (C-C motif) ligand 19 (CCL19), Chemokine (C-C motif) ligand 21 (CCL21), Galectin 3, Receptor for advanced glycation end-products (RAGE), Epithelial neutrophil-activating protein 78 (ENA78), Granulocyte-macrophage colony-stimulating factor (GM-CSF), Cluster of differentiation 30 (CD30), chemokine receptor 7 (CCR7), chondroitin sulfate proteoglycan 2 (CSPG2), IQ motif-containing GTPase activation protein 1 (IQGAP1), orosomucoid 1 (ORM1), arginase 1 (ARG1), lymphocyte antigen 96 (LY96), matrix metalloproteinase 9 (MMP9), carbonic anhydrase 4 (CA4), s100 calcium binding proteinA12 (s100A12), toll-like receptor 2 (TLR2), toll-like receptor 4 (TLR4), myeloid differentiation primary response gene 88 (MYD88), Janus Kinase 2 (JAK2), cluster of differentiation 3 (CD3), cluster of differentiation 4 (CD4), spleen tyrosine kinase (SYK), A kinase anchor protein 7 (AKAP7), CCAAT/enhancer binding protein (CEBPB), interleukin 10 (IL10), interleukin 8 (IL8), interleukin 22 receptor (IL22R) or an active fragment thereof. In some cases, the second class of biomolecules can be polypeptides. In some cases, polypeptides can comprise at least one of CCL19, CCL21, Galectin 3, RAGE, ENA78, GMCSF, CD30, CCR7, CSPG2, IQGAP1, ORM1, ARG1, LY96, MMP9, CA4, s100A12, Nav3, SAA, IGα, IGγ, IGκ, IGλ, or an active fragment thereof. In some cases a first class of biomolecules can comprise polynucleotides encoding one or more cytokines, and/or wherein a second class of biomolecules can comprise one or more cytokines. In some cases, one or more cytokines can comprise BAFF, MMP9, APP, Aggrecan, Galectin 3, Fas, RAGE, Ephrin A2, CD30, TNR1, CD27, CD40, TNFα, IL6, IL8, IL10, IL1β, IFNγ, RANTES, IL1α, IL4, IL17, IL2, GMCSF, ENA78, IL5, IL12P70, TARC, GroAlpha, IL33, BLCBCA, IL31, MCP2, IGG3, IGG4, Isoform 2 of Teneurin 1, and isoform 2 of aDisintegrin or any active fragment thereof. In some cases, a first group of biomarkers can be mRNA. In some cases, probes can be antibodies capable of binding at least one of a second group of biomarkers of ischemic stroke. In some cases, probes can be labelled with fluorochromes or radioactive isotopes.

### INCORPORATION BY REFERENCE

All publications, patents, and patent applications mentioned in this specification are herein incorporated by reference in their entireties to the same extent as if each individual publication, patent, or patent application was specifically and individually indicated to be incorporated by reference in their entireties.

### BRIEF DESCRIPTION OF THE DRAWINGS

The novel features described herein are set forth with particularity in the appended claims. A better understanding of the features and advantages of the features described herein will be obtained by reference to the following detailed description that sets forth illustrative examples, in which the principles of the features described herein are utilized, and the accompanying drawings of which:

FIGS. 1A-1D depict the mRNA expression of genes in the ischemic stroke group, the transient ischemic attack (TIA) group, and the stroke mimic group. FIG. 1A depicts the mRNA expression of ARG1. FIG. 1B depicts the mRNA expression of CCR7. FIG. 1C depicts the mRNA expression of LY96. FIG. 1D depicts the mRNA expression of CSPG2.

FIGS. 2A-2D depicts the mRNA expression of genes in the ischemic stroke group and the TIA group. FIG. 2A depicts the mRNA expression of IQGAP1. FIG. 2B depicts the mRNA expression of LY96. FIG. 2C depicts the mRNA expression of MMP9. FIG. 2D depicts the mRNA expression of s100a12.

FIG. 3 depicts the interaction among ARG1, CCR7, LY96, CSPG2, MMP9 and s100a12 across the ischemic stroke, the stroke mimic group, and the TIA group.

FIGS. 4A-4B depict the ratios of the mRNA expression of genes in the ischemic stroke group, the TIA group, and the stroke mimic group. FIG. 4A depicts the ratio between the mRNA expression of CCR7 and LY96. FIG. 4B depicts the ratio between the mRNA expression of MMP9 and s100a12.

FIGS. 5A-5B depict the ratios of the mRNA expression of genes in the ischemic stroke group and the TIA group. FIG. 5A depicts the ratio between the mRNA expression of MMP9 and s100a12. FIG. 5B depicts the ratio between the mRNA expression of ARG1 and s100a12.

FIGS. 6A-6D depict the genomic expression of genes in the ischemic stroke group and the metabolic disease control group. FIG. 6A depicts the genomic expression of ARG1. FIG. 6B depicts the genomic expression of MMP9. FIG. 6C depicts the genomic expression of s100a12. FIG. 6D depicts the genomic expression of CCR7.

FIG. 7 depicts the interaction among ARG1, MMP9, and s100a12 in the ischemic stroke group and the metabolic disease control group.

FIGS. 8A-8B depict protein expression in the ischemic stroke group, the TIA group and the stroke mimic group. FIG. 8A depicts the protein expression of ARG1. FIG. 8B depicts the protein expression of LY96.

FIGS. 9A-9B depict the ratios of the protein expression in the ischemic stroke group, the TIA group, and the stroke mimic group. FIG. 9A depicts the ratio between LY96 and ARG1. FIG. 9B depicts the ration between LY96 and CCR7.

FIGS. 10A-10H depict the results of whole proteomic scan of blood samples in the ischemic stroke group and the TIA group. FIG. 10A depicts the proteins whose expression levels are different between the ischemic stroke group and the TIA group. FIGS. 10B and 10C depict expression differences between stroke and TIA in males and females. FIGS. 10D-10H depict the transcriptional markers most associated with the proteins found to be different between male and female; hepatocyte nuclear factor 4 accounts for roughly 26% of the transcribed targets. In addition, complement and coagulation cascades are the most highly expressed, as 30% of the markers are involved in these pathways.

FIGS. 11A-11E depict the protein expression of cytokines in the ischemic stroke group, the TIA group, and the stroke mimic group. FIG. 11A depicts the protein expression of MMP9. FIG. 11B depicts the protein expression of Galectin 3. FIG. 11C depicts the protein expression of ENA78. FIG. 11D depicts the protein expression of RAGE. FIG. 11E depicts the protein expression of GMCSF.

FIGS. 12A-12B depict the protein expression of cytokines in the ischemic stroke group and the TIA group. FIG. 12A depicts the protein expression of Galectin 3. FIG. 12B depicts the protein expression of RAGE.

FIG. 13 depicts the interaction among MMP9, RAGE, and ENA7 in in the ischemic stroke group, the TIA group, and the stroke mimic group.

FIGS. 14A-14D depict the blood profile in the ischemic stroke group, the TIA group, the hemorrhagic stroke group, the traumatic brain injury (TBI) group, and the stroke mimic group. FIG. 14A depicts the white blood cell counts. FIG. 14B depicts the prothrombin times. FIG. 14C depicts the hematocrit percent. FIG. 14D depicts the troponin-1 concentrations.

FIGS. 15A-15B depict the blood profile in the ischemic stroke group, the TIA group, the hemorrhagic stroke group, and the stroke mimic group. FIG. 15A depicts neutrophil percentages. FIG. 15B depicts the white blood cell counts.

FIGS. 16A-16B depict the lymphocyte counts and neutrophil lymphocyte ratios in the ischemic stroke group, the TIA group, the hemorrhagic stroke group, the TBI group and the stroke mimic group. FIG. 16A depicts the lymphocyte counts and FIG. 16B depicts the neutrophil lymphocyte ratios.

FIGS. 17A-17H depict the correlations between time from ischemic stroke symptom onset and biomarkers at select time points. FIG. 17A depicts MYD88 expression. FIG. 17B depicts JAK2 expression. FIG. 17C depicts CD3 expression. FIG. 17D depicts SYK expression. FIG. 17E depicts CEBPB expression. FIG. 17F depicts IL10 expression. FIG. 17G depicts CA4 expression. FIG. 17H depicts CCR7 expression.

FIG. 18 depicts the correlations between time of ischemic stroke symptom onset and select biomarkers (Fas Ligand expression).

FIGS. 19A-19B depict the correlation between time of ischemic stroke symptom onset and select proteomic biomarkers. FIG. 19A depicts IGG3 expression. FIG. 19B depicts IGG4 expression.

FIGS. 20A-20B depict the correlation between time of ischemic stroke symptom onset and select immune biomarkers. FIG. 20A depicts CK-MB levels. FIG. 20B depicts Platelet counts.

FIG. 21 depicts an exemplary method for assessing ischemic stroke in a subject.

FIG. 22 depicts the use of GA-kNN for the identification of genes with strong discriminatory ability.

FIGS. 23A-23B show top 50 peripheral blood transcripts identified by GA-kNN for identification of AIS. FIG. 23A shows the top 50 peripheral blood transcripts ranked by GA-kNN based on their ability to discriminate between discovery cohort AIS patients and neurologically asymptomatic controls, ordered by the number of times each transcript was selected as part of a near-optimal solution. FIG. 23B shows differential peripheral blood expression of the top 50 transcripts between discovery cohort AIS patients and neurologically asymptomatic controls.

FIGS. 24A-24D show peripheral blood transcripts identified by GA-kNN displayed a strong ability to diagnose AIS in the discovery cohort. FIG. 24A shows a combination of the top ten ranked transcripts identified by GA-kNN (ANTXR2, STK3, PDK4, CD163, MAL, GRAP, ID3, CTSZ, KIF1B, and PLXDC2) were adequate to classify 98.4% of subjects in the discovery cohort correctly with a sensitivity of 97.4% and specificity of 100%. FIGS. 24B, 24C and 24D show the coordinate expression levels of the top ten ranked transcripts observed in discovery cohort neurologically asymptomatic controls and their AIS counterparts. AIS patients displayed a different pattern expression across the top ten markers in comparison to controls.

FIGS. 25A-25D show that the top 10 transcriptional markers identified in the discovery cohort demonstrated a strong ability to differentiate between AIS patients and controls in the validation cohort. FIG. 25A shows peripheral blood differential expression of the top ten transcripts between validation cohort AIS patients and neurologically asymptomatic controls. FIG. 25B shows that when comparing AIS patients to neurologically asymptomatic controls in the validation cohort, the top 10 transcripts used in combination were able to correctly identify 95.6% of subjects with a sensitivity of 92.3% and a specificity of 100%. FIG. 25C shows peripheral blood differential expression of the top ten transcripts between validation cohort AIS patients and stroke mimics. FIG. 25D shows when comparing AIS patients to stroke mimics in the validation cohort, the top 10 transcripts used in combination were able to correctly identify 96.3% of subjects with a specificity of 97.4% and a sensitivity of 93.3%.

FIGS. 26A-26D depict paradigm used for detection of plasma cfDNA using qPCR. FIG. 26A shows primers used for generation of the GFP605 spike-in control. FIG. 26B shows post-purification electrophoresis of purified GFP605. FIG. 26C shows primers designed for the detection of TERT and the GFP605 spike in control. FIG. 26D shows PCR products generated using primers designed to target TERT and the 108 bp internal fragment of GFP605 using total human DNA, purified GFP605 spike-in, or a combination of both as template.

FIG. 27 shows patient clinical and demographic characteristics.

FIGS. 28A and 28B depict circulating cfDNA levels in AIS patients and stroke mimics. FIG. 28A shows comparison of circulating cfDNA levels between AIS patients and stroke mimics. FIG. 28B shows sensitivity and specificity of circulating cfDNA levels as an identifier of AIS when discriminating between AIS patients and stroke mimics.

FIGS. 29A and 29B depict relationship between circulating cfDNA levels and injury severity in AIS patients. FIG. 29A shows relationship between circulating cfDNA levels and NIHSS. FIG. 29B shows relationship between circulating cfDNA levels and infarct volume.

FIG. 30 shows relationship between circulating cfDNA levels and neutrophil count in AIS patients.

### DETAILED DESCRIPTION

### Overview

Provided herein are methods for assessing ischemic stroke in a patient. The methods can comprise measuring a level of cell-free nucleic acids (e.g., cell-free DNA) in a body fluid (e.g., blood) obtained from a patient. The level of the cell-free nucleic acids in the body fluid can be compared to a reference value, e.g., a level of cell-free nucleic acids in the body fluid from a healthy individual or stroke mimics. Ischemic stroke can be detected in the patient if the level of the cell-free nucleic acids in the body fluid is higher (e.g., 3-fold higher) than the reference value. In some cases, the methods disclosed herein can distinguish ischemic stroke from ischemic mimics with a sensitivity of at least 86% and a specificity of at least 75%. In some cases, the level of cell-free nucleic acids is an indicator of the status of innate immune system activation by stroke (e.g., represented by peripheral blood neutrophil count), or an indicator of the severity of injury caused by the stroke. In some cases, as the level of cell-free nucleic acids increases stroke severity increases. In some cases, as the level of cell-free nucleic acids increases stroke severity decreases. The methods for assessing ischemic stroke can comprise measuring a level of cell-free nucleic acids carrying one or more epigenetic markers. The level of the cell-free nucleic acids with the epigenetic markers can be compared to a reference level. In some cases, a ratio of the cell-free nucleic acids carrying an epigenetic marker to the total cell-free nucleic acids in the sample is calculated. In some embodiments, ischemic stroke can be assessed based on the ratio.

Also provided herein are devices for performing the methods for assessing ischemic stroke in a patient. Such device can comprise a memory that stores executable instructions, and a processor that executes the executable instructions to perform the method described herein. In some cases, the devices are portable devices. For example, the devices can be point-of-care devices that are used to rule-in or rule-out ischemic stroke and the severity of the stroke to aid in transportation and triage of patients to stroke certified centers, facilitate early administration of thrombolytic therapy or in the cases of no-stroke, appropriate follow up care.

The methods for assessing ischemic stroke can include any combination of the methods described throughout this disclosure. For example, the methods for assessing ischemic stroke can comprise one or more of: a) measuring a gene profile in a patient, b) measuring an RNA profile in a patient, c) measuring a protein profile in the patient, d) measuring expression (e.g., at a mRNA level, a protein level, or both) of a group of biomarkers disclosed herein, e) measuring cell-free nucleic acid levels in a body fluid in the patient, e) other assessment of stroke, including neuropathological imaging and measuring a blood profile of blood cells in the patient.

Provided herein are methods, devices and kits for assessing ischemic stroke in a subject (e.g., a subject suspected of having ischemic stroke). The methods of accessing ischemic stroke in a subject can comprise measuring the expression of a group of biomarkers in a sample from a subject, comparing the expression of the group of biomarkers to a reference, and assessing ischemic stroke in a subject (e.g., using a computer system). The methods provided herein can comprise measuring expression of two or more (e.g., two, three, four, five, six, seven, eight, nine or ten) biomarkers comprising for example anthrax toxin receptor 2, serine/threonine-protein kinase 3, pyruvate dehydrogenase lipoamide kinase isozyme 4, cluster of differentiation 163, myelin and lymphocyte protein, GRB2-related adaptor protein, inhibitor of DNA binding 3, cathepsin Z, kinesin-like protein 1B, and plexin domain-containing protein 2.

The methods, devices and kits provided herein can achieve a specificity of at least about 96% and a sensitivity of at least about 96% in assessing ischemic stroke. In some cases, the methods, devices and kits can achieve a specificity of at least about 96% and a sensitivity of at least about 96% in assessing ischemic stroke based on the expression of two biomarkers. In some cases, the methods, devices and kits can achieve a specificity of about 100% and a sensitivity of about 100% in assessing ischemic stroke based on the expression of four biomarkers.

The kits provided herein can comprise a panel of probes for measuring the expression of two or more (e.g., two, three, four, five, six, seven, eight, nine, or ten) biomarkers in a sample from a subject. The probes can be used for measuring the expression of two or more (e.g., four, five, six, seven, eight, nine or ten) of for example anthrax toxin receptor 2, serine/threonine-protein kinase 3, pyruvate dehydrogenase lipoamide kinase isozyme 4, cluster of differentiation 163, myelin and lymphocyte protein, GRB2-related adaptor protein, inhibitor of DNA binding 3, cathepsin Z, kinesin-like protein 1B, and plexin domain-containing protein 2.

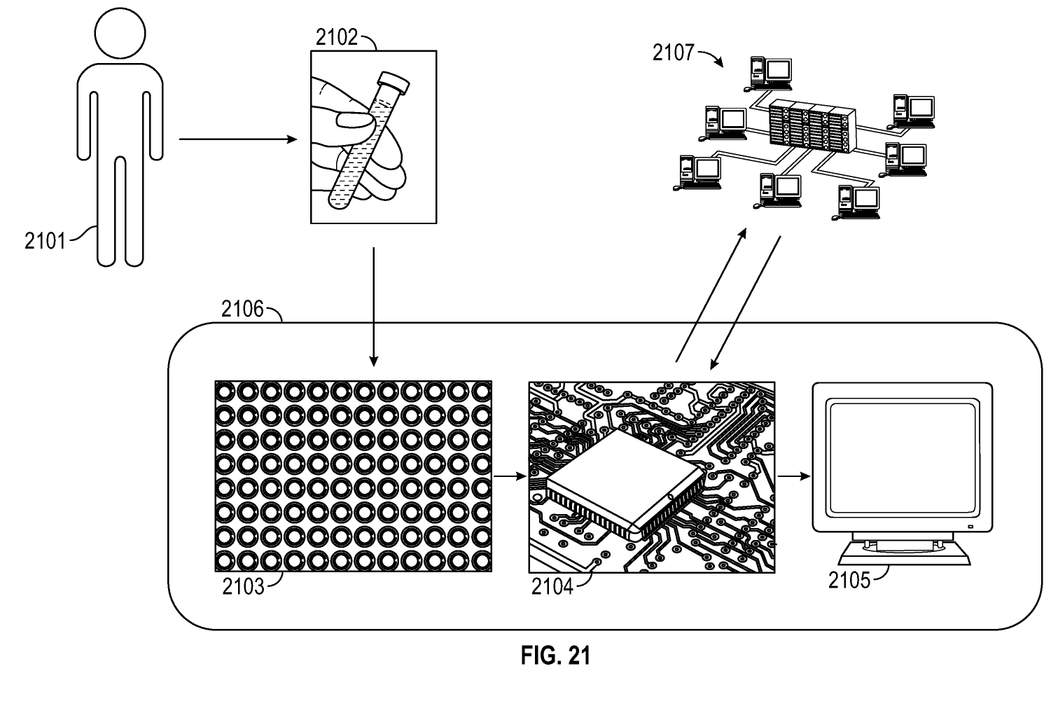

FIG. 21 shows an exemplary method for assessing ischemic stroke in a subject. Peripheral blood (FIG. 21, 2102) can be drawn from a subject (FIG. 21, 2101). The expression of a group of biomarkers in the blood can be measured by an assay (FIG. 21, 2103). In some cases, the assay can be a protein-based assay, such as enzyme-linked immunosorbent assay (ELISA). In some cases, the assay can be a nucleic acid-based assay, such as an assay involving nucleic acid amplification. Exemplary nucleic acid-based assays include polymerase chain reaction (PCR), e.g., quantitative reverse transcription PCR (q-RT PCR). In some cases, both protein and RNA expression of the group of biomarkers can be measured for assessing ischemic stroke. In further cases, other assays such as blood cell profile assays can be used in combination with the expression of biomarkers for assessing ischemic stroke. The expression levels of the group of biomarkers can be analyzed by a computer system (FIG. 21, 2104). In some cases, the computer system can compare the expression of the biomarkers to a reference. The reference can be stored in the computer system. Alternatively, the reference can be stored in other computers, databases, and/or servers, and accessible through a network (e.g. Internet) (FIG. 21, 2107). The result of whether a subject has ischemic stroke can be transmitted to an output device, e.g., a monitor (FIG. 21, 2105). The assay, the computer system, and the output device (FIGS. 21, 2103, 2104 and 2105) can be integrated into a single device (FIG. 21, 2106). In some cases, such device can be a point of care device, e.g., a portable point of care device. In some cases, the computer system can be a smartphone.

Provided herein include methods for identifying one or more biomarkers of ischemic stroke. The methods can comprise measuring a profile of polynucleotides in an ischemic stroke sample and a profile of polypeptides in the same or a different ischemic stroke sample, and analyzing the profiles by comparing the profile of polynucleotides and/or the profile polypeptides to reference profiles. The analyzing can identify biomarkers that have different expression levels under an ischemic stroke condition compared to a non-ischemic stroke condition. In addition, the analyzing can also determine a plurality of biomarkers that have different expression patterns under an ischemic stroke condition compared to a non-ischemic stroke condition. One or more polynucleotides and the polypeptides encoded by the one or more polynucleotides can be identified as biomarkers of ischemic stroke if the expression levels of both the polynucleotides and the polypeptides are increased or decreased under an ischemic stroke condition compared to their expression levels under a non-ischemic stroke condition.

Provided herein include methods, devices and kits for detecting ischemic stroke by evaluating the profiles (e.g., expression level) of biomarkers in a biological sample. The methods can allow for the detection of ischemic stroke in a timely manner, which can be critical for effective treatments. Such methods can comprise measuring the expression pattern of a group of one or more polynucleotide biomarkers of ischemic stroke and the expression pattern of a group of one or more adaptamer biomarkers of ischemic stroke in a subject, analyzing the expression patterns and detecting ischemic stroke in the subject. Such methods can comprise measuring the expression pattern of a group of one or more polynucleotide biomarkers of ischemic stroke and the expression pattern of a group of one or more adaptamer biomarkers of ischemic stroke in a subject, analyzing the expression patterns and detecting ischemic stroke in a subject. Such methods can comprise measuring the expression pattern of a group of one or more polynucleotide biomarkers of ischemic stroke and the expression pattern of a group of one or more polypeptide biomarkers of ischemic stroke in a subject, analyzing the expression patterns and detecting ischemic stroke in the subject. The expression patterns of the biomarkers can be used to distinguish ischemic stroke from non-ischemic stroke, traumatic brain injuries and/or stroke mimics, which can be important for selecting suitable treatment for a subject. The expression patterns of the biomarkers can also be used to determine the time of ischemic stroke onset. The expression patterns of the biomarkers can also be used to predict ischemic stroke outcome. The expression patterns of the biomarkers can also be used to predict ischemic stroke severity. In some cases, the methods can be used to detect ischemic stroke within about 4.5 hours. Diagnosis of ischemic stroke within about 4.5 hours can enhance the effectiveness of stroke treatments (e.g., tissue plasminogen activator (tPA)). In some aspects, the expression patterns of biomarkers can be used to measure the effectiveness of treatment. In some aspects, the expression patterns of biomarkers can be measured before, during, or after treatment. The expression patterns of biomarkers of ischemic stroke can be measured by an enzyme-linked immunosorbent assay (ELISA), bead-based multiplex assay, microarray, mass spectrometry or any other assays that can be performed in a time-sensitive and/or bedside manner.

Also provided herein include kits for detecting ischemic stroke in a subject. The kits can comprise a first panel of probes for detecting one or more polynucleotide biomarkers of ischemic stroke and a second panel of probes for detecting one or more polypeptide biomarkers of ischemic stroke. Probes for detecting polynucleotide biomarkers can be oligonucleotides capable of hybridizing to the polynucleotide biomarkers. Probes for detecting polypeptide biomarkers can be antibodies capable of binding to the polypeptide biomarkers. In some cases, probes can be labeled (e.g., with a fluorochrome) to provide a detectable signal used in ischemic stroke diagnosis.

Further disclosed herein include devices for detecting ischemic stroke in a subject. The devices can be a computer system. A device can comprise a memory that stores executable instructions and a processor to execute the executable instructions to perform any methods for detecting ischemic stroke. In some cases, a device can detect biomarkers of ischemic stroke in a subject using probes in kits disclosed herein. In some aspects, devices can detect ischemic stroke. A device can be contemplated to be portable devices for use in a hospital and/or a pre-hospital setting (e.g., in an ambulance or patient's home). In some cases, a device can be filament-based devices.

### Methods

Provided herein are methods of assessing ischemic stroke in a subject (e.g., a subject suspected of having ischemic stroke).

The methods disclosed herein can distinguish ischemic stroke from stroke mimic. In some cases, one of such methods comprises one or more steps of a) measuring a level of cell-free nucleic acids in a sample from a subject; b) comparing the level of cell-free nucleic acids to a reference level of cell-free nucleic acids in a reference sample, wherein the reference sample is from a stroke mimic subject; and c) determining whether the sample or the reference sample has a higher level of cell-free nucleic acids.

The methods disclosed herein can assess stroke (e.g., ischemic stroke) with high specificity and sensitivity. In some case, one of such methods comprise one or more steps of a) measuring a level of cell-free nucleic acids in a sample from a subject; b) comparing the level of cell-free nucleic acids to a reference level of cell-free nucleic acids in a reference sample, wherein the reference sample is from a non-ischemic stroke subject; and c) assessing ischemic stroke in the subject using a computer system, wherein the assessing can differentiate ischemic stroke from non-ischemic stroke with a sensitivity of at least about 80% and a specificity of at least about 75%.

The methods disclosed herein can assess ischemic stroke based on the level of cell-free nucleic acids carrying one or more epigenetic markers. In some cases, one of such methods comprises one of more steps of a) measuring a level of cell-free nucleic acids carrying an epigenetic marker, wherein the cell-free nucleic acids are in a sample from a subject suspected of having an ischemic stroke, b) comparing the level of the cell-free nucleic acids to a reference level of cell-free nucleic acids carrying the epigenetic marker in a reference sample, wherein the reference sample is from a healthy control subject or a stroke mimic subject. The methods can also assess ischemic stroke based on the ratio of the cell-free nucleic acids carrying an epigenetic marker to the total cell-free nucleic acids level in a sample. In some aspects, a ratio of the cell-free nucleic acids carrying an epigenetic marker to the total cell-free nucleic acids in a sample can be in a range from about 0.01 to about 10000. In some aspects, a ratio of cell-free nucleic acids carrying an epigenetic marker to total cell-free nucleic acids in a sample can be at least about 0.0005, 0.5, 1, 1.5, 2, 3, 4, 5, 6, 7, 8, 9, 10, 20, 30, 40, 50, 60, 70, 80, 90, 100, 200, 500, or at least about 1000. In some aspects, a ratio of the total cell-free nucleic acids in a sample to cell-free nucleic acids carrying an epigenetic marker can be at least about 0.0005, 0.5, 1, 1.5, 2, 3, 4, 5, 6, 7, 8, 9, 10, 20, 30, 40, 50, 60, 70, 80, 90, 100, 200, 500, or at least about 1000. One of such methods can comprise one or more steps of: a) measuring a level of cell-free nucleic acids in a sample from a subject suspected of having an ischemic stroke; b) measuring a level of a subgroup of the cell-free nucleic acids, wherein the subgroup of the cell-free nucleic acids carry an epigenetic marker; c) determining a ratio between the level of cell-free nucleic acids and the level of the subgroup of the cell-free nucleic acids; d) comparing the ratio to a reference, wherein the reference is a ratio between a level of cell-free nucleic acids in a reference sample and a level of a subgroup of the cell-free nucleic acids in the reference sample, wherein the subgroup of the cell-free nucleic acids in the reference sample carry the epigenetic marker, and wherein the reference sample is from a healthy control subject or a stroke mimic subject. In some cases, ischemic stroke is detected in a subject if a ratio of cell-free nucleic acids carrying an epigenetic marker to total cell-free nucleic acids in a sample is higher than a ratio between a level of cell-free nucleic acids in a reference sample and a level of a subgroup of cell-free nucleic acids in a reference sample, wherein a subgroup of the cell-free nucleic acids in a reference sample carry the epigenetic marker. In some cases, ischemic stroke is not detected in a subject if a ratio of cell-free nucleic acids carrying an epigenetic marker to total cell-free nucleic acids in a sample is higher than a ratio between a level of cell-free nucleic acids in a reference sample and a level of a subgroup of cell-free nucleic acids in the reference sample, wherein the subgroup of the cell-free nucleic acids in the reference sample carry the epigenetic marker.

In some embodiments, a level of cell-free nucleic acids can determine infarct volume in a subject. In some embodiments, as a level of cell-free nucleic acids increase infarct volume increases. In some embodiments, as a level of cell-free nucleic acids decrease infarct volume increases. In some embodiments, a higher level of cell-free nucleic acids correlates with a larger infarct volume. In some embodiments, a lower level of cell-free nucleic acids correlates with a smaller infarct volume.

Any step of the methods herein can be performed using a computer system. A computer system can comprise a memory that stores executable instructions and a processor to execute the executable instructions to perform any step of the methods herein. In some cases, one or more of the assessing steps herein can be performed using a computer system.

The methods can comprise measuring a level of cell-free nucleic acids in a sample from a subject. Any conventional DNA or RNA detection methods can be used for measuring the cell-free nucleic acids. Measuring cell-free nucleic acids can comprise detection of amount, concentration, or both of the cell-free nucleic acids. In some cases, any means for detecting low copy number nucleic acids can be used to detect the nucleic acids. Methods for detecting and quantifying low copy number nucleic acids include analytic biochemical methods such as electrophoresis, capillary electrophoresis, high performance liquid chromatography (HPLC), thin layer chromatography (TLC), hyperdiffusion chromatography, mass spectroscopy, spectrophometry, electrophoresis (e.g., gel electrophoresis), and the like. Measuring the level of cell-free nucleic acids can be performed using a polymerase chain reaction (PCR), e.g., any PCR technology described in the disclosure. In some cases, the level of cell-free nucleic acids can be measured by quantitative PCR (e.g., quantitative real-time PCR).

Measuring the level of cell-free nucleic acids can be performed by measuring the level of one or more markers (one or more genes or fragments thereof) whose level is indicative of the level of cell-free nucleic acids in the sample. In some cases, such markers can be present in ischemic stroke subject at a higher level compared to a healthy or stroke mimic subject. The level of cell-free nucleic acids can be measured by detecting the level of human leukocyte antigen (HLA) locus, mitochondrial DNA, mitochondrial RNA (e.g., mitochondrial mRNA), Y chromosomal genes blood group antigen genes like RHD (cluster of differentiation 240D (CD240D)), ribonuclease P RNA component H1, Alu J element, endogenous retrovirus group 3, glyceraldehyde 3-phosphate dehydrogenase, N-acetylglucosamine kinase, alcohol dehydrogenase, beta-globin, a member of the albumin family, telomerase reverse transcriptase (TERT), or any combination thereof. Detection of the level of these markers include the detection the level of the gene (or a fragment thereof), or transcripts, e.g., mRNA (or a fragment thereof) of the markers. In some cases, such a marker can be TERT.

Measuring the level of cell-free nucleic acids can be performed using a probe. Similarly, measuring the level of cell-free nuclei acids carrying one or more epigenetic markers can be performed using a probe. A probe can bind (e.g., directly or indirectly) to at least one of the cell-free nucleic acids, or at least one of the cell-free nucleic acids carrying one or more epigenetic markers. In some cases, a probe can be labeled. Such probes and labels are disclosed herein. In some cases, a probe can be a polynucleotide. For example the polynucleotide can hybridize with at least one of the cell-free nucleic acids in the sample. In some embodiments, a polynucleotide can be double stranded or single stranded.

When measuring a level of cell-free nucleic acids in a sample, a polynucleotide can be added into the sample as a control (e.g. Exogenous polynucleotide). The level of the exogenous polynucleotide can be indicative of loss or bias during nucleic acid manipulation steps (e.g., isolation, purification or concentration). For example, when isolating or purifying nucleic acid from a sample, the isolating or purification efficiency can be determined by comparing the level of the polynucleotide before and after the isolation or purification step. In some cases, such polynucleotide is one of a nucleic acid in the sample (e.g., an endogenous polynucleotide). In some cases, such polynucleotide does not exist in the sample, e.g., an exogenous polynucleotide. An exogenous polynucleotide can be synthetic or from another species different from the subject being tested. In some case, an exogenous polynucleotide is a fluorescence protein (e.g., green fluorescent protein (GFP)) or a fragment thereof. For example, an exogenous polynucleotide can be a fragment of a DNA fragment (e.g., a 605 bp fragment) originating from the GFP-encoding portion of the pontellina plumata genome.

In some embodiments, after measuring a level of cell-free nucleic acids in a sample in a subject, a level of cell-free nucleic acids in a sample can be compared to a reference. A reference can be a level of cell-free nucleic acids in a reference sample from any reference subject described in this disclosure, e.g., a healthy subject or a stroke mimic subject.

Ischemic stroke can be assessed based on comparison of cell-free nucleic acid with a reference. In some cases, ischemic stroke is detected in a subject if a level of the cell-free nucleic acids is increased compared to a reference. For example, ischemic stroke is detected in a subject if a level of cell-free nucleic acids is increased by at least about 1%, 5%, 7%, 10%, 20%, 40%, 60%, 80%, 1 fold, 2 fold, 3 fold, 4 fold, 5 fold, 10 fold, or 20 fold compared to a reference. Alternatively, ischemic stroke can be detected in a subject if a level of cell-free nucleic acids is decreased compared to a reference. For example, ischemic stroke is detected in a subject if a level of cell-free nucleic acids is decreased by at least about 1%, 5%, 7%, 10%, 20%, 40%, 60%, 80%, 1 fold, 2 fold, 3 fold, 4 fold, 5 fold, 10 fold, or 20 fold compared to a reference.

The methods herein can comprise measuring a level of cell-free nucleic acids that carry one or more epigenetic markers. In some cases, cell-free nucleic acids carrying one or more epigenetic markers are a subgroup of cell-free nucleic acids in a sample from a subject. In some cases, a subgroup of cell-free nucleic acids can comprise a gene or a fragment thereof carrying an epigenetic marker. In some cases, the subgroup of cell-free nucleic acids can be a plurality of genes or fragments thereof that carry an epigenetic marker. The subgroup of cell-free nucleic acids can carry more than one epigenetic marker.

An epigenetic marker can include one or more of acetylation, methylation, ubiquitylation, phosphorylation, sumoylation, ribosylation, citrullination of a polynucleotide. In some cases, an epigenetic modification can include histone modification, including acetylation, methylation, ubiquitylation, phosphorylation, sumoylation, ribosylation, or citrullination of a histone.

A subgroup of cell-free nucleic acids can be RNA transcripts specific for one or limited types of cells or tissues. For example, the subgroup of cell-free nucleic acids can be RNA that is only or predominantly transcribed in one or a limited types of cells or tissues. Such RNA can be mRNA or microRNA. In some cases, the subgroup of cell-free DNA can be mRNA transcripts specific to cells from a neurovascular unit in a subject.

A sample can be obtained from a subject after the subject exhibits a stroke symptom (e.g., an ischemic stroke symptom). For example, a sample can be obtained from a subject at least about 0.5, 1, 1.5, 2, 2.5, 3, 3.5, 4, 4.5, 5, 5.5, 6, 6.5, 7, 7.5, 8, 10, 12, 15, 20, 24, 50, 72, 96, or 120 hours from the onset of a stroke symptom (e.g., an ischemic stroke symptom).

Assessing stroke (e.g., ischemic stroke) in a subject can comprise one or more of the following: a) determining whether the subject has a stroke (e.g., ischemic stroke); b) assessing the risk of the subject for having a stroke (e.g., ischemic stroke); c) assessing the stroke severity in the subject; d) predicting the stroke severity in the subject; e) assessing the activation of innate immune system (e.g., assessing the neutrophil count in the subject); and f) assessing a stroke-induced injury (e.g., myocardial infarction). One or more of assessment can be performed based on the level of cell-free nucleic acids. For example, neutrophil count can be determined based on the level of cell-free nucleic acids in the sample.

The level of cell-free nucleic acids can be compared to a reference level. The reference level can be the level of cell-free nucleic acids in a reference sample. A reference sample can be a sample taken from a healthy subject. A reference sample can be a sample taken from a non-stroke subject. For example, a reference sample can be a sample taken from a subject with a stroke mimic. In some cases, a reference can be stored in a database or on a server.

The methods disclosed herein can comprise determining a time of ischemic stroke symptom onset in a subject. In some cases, a time of ischemic stroke symptom onset can be determined by correlating the level of cell-free nucleic acids in a sample with the time of ischemic stroke symptom onset. Prior to this invention, the determination of time of stroke symptom onset was often difficult and inaccurate, and especially when patients are severely comprised or the events are un-witnessed. These problems are due in part to limitations in the technology currently used to evaluate a patient for when their stroke began (clinician and patient/surrogate interaction) and limitations in the level of experience and/or proper training possessed by medical clinicians who engage the patients. These circumstances are detrimental to stroke and brain injury victims because accurate, nonbiased prediction of time of stroke onset is extremely important to the health and outcome of the patients at the point of care. tPA was approved by FDA in 1996 for treating stroke within 3 hours of symptom onset and remains the only FDA approved medication indicated for the treatment of acute ischemic stroke. Currently the AHA/ASA has published a science advisory endorsing the extension of this time window to within 4.5 hours of symptom onset. The present invention is related to methods for determining the onset of stroke symptoms.

Provided herein are methods of assessing ischemic stroke in a subject (e.g., a subject suspected of having ischemic stroke). The methods can comprise measuring expression of a group of biomarkers in a sample from a subject. The expression can then be compared to a reference. Ischemic stroke in the subject can then be assessed based on the expression (e.g., using a computer system). The expression can be RNA expression, protein expression, or a combination thereof.

The provided methods increase the accuracy of diagnosing stroke. The provided methods and the inventions disclosed herein provide increased specificity and specificity.

Provided herein include methods for identifying biomarkers of ischemic stroke. The methods can comprise measuring a profile of polynucleotides in a first ischemic stroke sample and measuring a profile of polypeptides in a second ischemic stroke sample. A first group of biomarkers can be identified by comparing the profile of polynucleotides in the first ischemic stroke sample to a polynucleotide reference profile. For example, a first group of biomarkers can include genes whose expression levels are up-regulated or down-regulated in a first ischemic stroke sample comparing to a polynucleotide reference profile. A second group of biomarkers can be identified by comparing a profile of polypeptides in a second ischemic stroke sample to a polypeptide reference profile. In some cases, a second group of biomarkers can include polypeptides whose expression levels are up-regulated or down-regulated in a second ischemic stroke sample compared to a polypeptides reference profile. The method can further comprise analyzing a first group of biomarkers and a second group of biomarkers, and identifying one or more biomarkers of ischemic stroke. For example, the one or more biomarkers can include genes whose mRNA expression levels and protein expression levels are up-regulated or down-regulated compared to the gene and protein expression levels in a non-ischemic stroke subject.

A sample can be obtained from an organism or from components (e.g., cells) of a subject. A sample can be of any biological tissue or fluid. A sample herein can include brain cells or tissues, cerebrospinal fluid, nerve tissue, sputum, blood, serum, plasma, blood cells (e.g., white cells), tissue samples, biopsy samples, urine, peritoneal fluid, and pleural fluid, saliva, semen, breast exudate, tears, mucous, lymph, cytosols, ascites, amniotic fluid, bladder washes, bronchioalveolar lavages or cells therefrom, among other body fluid samples, and combinations thereof. A sample can be a body fluid. The body fluid can comprise cell-free nucleic acids. Such body fluid can be any fluidic sample described herein. For example a body fluid can be blood or a fraction thereof. In some cases, a body fluid is plasma. In some cases, a body fluid is serum.

A cell-free nucleic acid can be any extracellular nuclei acid that is not attached to a cell. A cell-free nucleic acid can be a nucleic acid circulating in blood. Alternatively, a cell-free nucleic acid can be a nucleic acid in other body fluid, e.g., urine. In some cases, a cell-free nucleic acid is DNA, e.g., genomic DNA, mitochondrial DNA, or a fragment thereof. In some cases, a cell-free nucleic acid is RNA, e.g., mRNA, siRNA, miRNA, cRNA, tRNA, rRNA, small nucleolar RNA (snoRNA), Piwi-interacting RNA (piRNA), long ncRNA, or a fragment thereof. A cell-free nucleic acid can be double stranded, single stranded, or a hybrid thereof. A cell-free nucleic acid can be released into body fluid through secretion or cell death processes, e.g., cellular necrosis and apoptosis.

The methods disclosed herein can comprise measuring cell-free nucleic acids that are specific to one or more types of cells or tissues. In some cases, a cell-free nucleic acid specific to a type of cell or tissue is exclusively or predominantly produced or derived from the type of cell or tissue. In some cases, cell-free nucleic acid specific to a type of cell or tissue is also produced or derived from other types of cells or tissues. For example, the cell-free nucleic acids can be specific to cells of a neurovascular unit. For example, the cell-free nucleic acids can be derived from a neutrophil extracellular trap.

In some cases, a neurovascular unit comprises a dynamic structure comprising one or more of endothelial cells, basal lamina, astrocytic foot processes, pericyte, microglia or neurons. In some cases, a neutrophil extracellular trap can comprises a network of extracellular fibers. In some cases, the extracellular fibers can comprise DNA. In some cases, the extracellular fibers can comprise DNA from neutrophils.

An epigenetic marker can be specific to one or more types of cells, or tissues. In some cases, an epigenetic marker can only or predominantly be carried by a gene from one or limited types of cells or tissues. In some cases, an epigenetic marker is specific to cells from a neurovascular unit in a subject.

The cell-free nucleic acids or epigenetic marker discussed above can be specific to one or more tissues, including brain, lung, liver, heart, spleen, pancreas, small intestine, large intestine, skeletal muscle, smooth muscle, skin, bones, adipose tissues, hairs, thyroid, trachea, gall bladder, kidney, ureter, bladder, aorta, vein, esophagus, diaphragm, stomach, rectum, adrenal glands, bronchi, ears, eyes, retina, genitals, hypothalamus, larynx, nose, tongue, spinal cord, or ureters, uterus, ovary, testis, and/or any combination thereof.

The cell-free nucleic acids or epigenetic marker discussed above can be specific to one or more types of cells, including trichocytes, keratinocytes, gonadotropes, corticotropes, thyrotropes, somatotropes, lactotrophs, chromaffin cells, parafollicular cells, glomus cells melanocytes, nevus cells, merkel cells, odontoblasts, cementoblasts corneal keratocytes, retina muller cells, retinal pigment epithelium cells, neurons, glias (e.g., oligodendrocyte astrocytes), ependymocytes, pinealocytes, pneumocytes (e.g., type I pneumocytes, and type II pneumocytes), clara cells, goblet cells, G cells, D cells, Enterochromaffin-like cells, gastric chief cells, parietal cells, foveolar cells, K cells, D cells, I cells, goblet cells, paneth cells, enterocytes, microfold cells, hepatocytes, hepatic stellate cells (e.g., Kupffer cells from mesoderm), cholecystocytes, centroacinar cells, pancreatic stellate cells, pancreatic α cells, pancreatic β cells, pancreatic δ cells, pancreatic F cells, pancreatic c cells, thyroid (e.g., follicular cells), parathyroid (e.g., parathyroid chief cells), oxyphil cells, urothelial cells, osteoblasts, osteocytes, chondroblasts, chondrocytes, fibroblasts, fibrocytes, myoblasts, myocytes, myosatellite cells, tendon cells, cardiac muscle cells, lipoblasts, adipocytes, interstitial cells of cajal, angioblasts, endothelial cells, mesangial cells (e.g., intraglomerular mesangial cells and extraglomerular mesangial cells), juxtaglomerular cells, macula densa cells, stromal cells, interstitial cells, telocytes simple epithelial cells, podocytes, kidney proximal tubule brush border cells, sertoli cells, leydig cells, granulosa cells, peg cells, germ cells, spermatozoon ovums, lymphocytes, myeloid cells, endothelial progenitor cells, endothelial stem cells, angioblasts, mesoangioblasts, pericyte mural cells, and/or any combination thereof.

A sample can be fresh or frozen, and/or can be treated, e.g. with heparin, citrate, or EDTA. A sample can also include sections of tissues such as frozen sections taken for histological purposes. In some cases, a sample can be an ischemic stroke sample. An ischemic stroke sample can be a sample derived from a subject with ischemic stroke or having a risk of having ischemic stroke. In some cases, an ischemic stroke sample can be a sample derived from a subject with an ischemic stroke. For example, an ischemic stroke sample can be a sample derived from a subject within a range of about 0.5 hours to about 120 hours of an ischemic stroke. In a particular example, an ischemic stroke sample can be a sample derived from a subject within about 0.5, 1, 1.5, 2, 2.5, 3, 3.5, 4, 4.5, 5, 5.5, 6, 6.5, 7, 7.5, 8, 10, 12, 15, 20, 24, 50, 72, 96, 120, 150, or 200 hours of an ischemic stroke.

In some cases, a sample can be a biological fluid. When a sample is a biological fluid, the volume of the fluidic sample can be greater than 1 mL (milliliter). In some cases, the volume of the fluidic sample can be within a range of about 1.0 mL to about 15 mL. For example, the volume of the sample can be about 1.0 mL, 1.1 mL, 1.2 mL, 1.4 mL, 1.6 mL, 1.8 mL, 1.9 mL, 2 mL, 3 mL, 4 mL, 5 mL, 6 mL, 7 mL, 8 mL, 9 mL, or 10 mL. Alternatively, in some cases, the volume of the fluidic sample can be no greater than 1 mL. For example, the volume of the sample can be less than 0.00001 mL, 0.0001 mL, 0.001 mL, 0.01 mL, 0.1 mL, 0.2 mL, 0.4 mL, 0.6 mL, 0.8 mL, 1 mL.

A sample disclosed herein can be blood. For example, a sample can be peripheral blood. In some cases, a sample can be a fraction of blood. In one example, a sample can be serum. In another example, a sample can be plasma. In another example, a sample can include one or more cells circulating in blood. Such cells can include red blood cells (e.g., erythrocytes), white blood cells (e.g., leukocytes, including, neutrophils, eosinophils, basophils, lymphocyte, and monocytes (e.g., peripheral blood mononuclear cell)), platelets (e.g., thrombocytes), circulating tumor cells, or any type of cells circulating in peripheral blood and combinations thereof.

A sample can be derived from a subject. In some cases, a subject can be a human, e.g. a human patient. In some cases, a subject can be a non-human animal, including a mammal such as a domestic pet (e.g., a dog, or a cat) or a primate. A sample can contain one or more polypeptide or protein biomarkers, or a polynucleotide biomarker disclosed herein (e.g., mRNA). A subject can be suspected of having a condition (e.g., a disease). For example, a subject can be suspected of having stroke (e.g., ischemic stroke).

Stroke can refer to a medical condition that occurs when the blood supply to part of the brain is interrupted or severely reduced, depriving brain tissue of oxygen and nutrients. Within minutes, brain cells can begin to die. Stroke can include ischemic stroke, hemorrhagic stroke and transient ischemic attack (TIA). Ischemic stroke can occur when there is a decrease or loss of blood flow to an area of the brain resulting in tissue damage or destruction. Hemorrhagic stroke can occur when a blood vessel located in the brain is ruptured leading to the leakage and accumulation of blood directly in the brain tissue. Transient ischemic attack or mini stroke, can occur when a blood vessel is temporarily blocked. Ischemic stroke can include thrombotic, embolic, lacunar and hypoperfusion types of strokes.

An ischemic stroke subject can refer to a subject with an ischemic stroke or having a risk of having an ischemic stroke. In some cases, an ischemic stroke subject can be a subject that has had ischemic stroke within 24 hours. In a particular example, an ischemic stroke subject can be a subject that has had an ischemic stroke within 4.5 hours. A non-ischemic stroke subject can be a subject who has not had an ischemic stroke. In some cases, a non-ischemic stroke subject can be a subject who has not had an ischemic stroke and has no risk of having an ischemic stroke.

A subject with stroke (e.g., ischemic stroke) can have one or more stroke symptoms. Stroke symptoms can be present at the onset of any type of stroke (e.g., ischemic stroke or hemorrhagic stroke). Stroke symptoms can be present before or after the onset of any type of stroke. Stroke symptoms can include those symptoms recognized by the National Stroke Association, which include: (a) sudden numbness or weakness of the face, arm or leg—especially on one side of the body; (b) sudden confusion, trouble speaking or understanding; (c) sudden trouble seeing in one or both eyes; (d) sudden trouble walking, dizziness, loss of balance or coordination, and (e) sudden severe headache with no known cause.

A non-ischemic stroke subject can have stroke-mimicking symptoms. Stroke-mimicking symptoms can include pain, headache, aphasia, apraxia, agnosia, amnesia, stupor, confusion, vertigo, coma, delirium, dementia, seizure, migraine insomnia, hypersomnia, sleep apnea, tremor, dyskinesia, paralysis, visual disturbances, diplopia, paresthesias, dysarthria, hemiplegia, hemianesthesia, and hemianopia. When a stroke-mimicking symptom is present in a subject that has not suffered a stroke, the symptoms can be referred to as “stroke mimics”. Conditions within the differential diagnosis of stroke include brain tumor (e.g., primary and metastatic disease), aneurysm, electrocution, burns, infections (e.g., meningitis), cerebral hypoxia, head injury (e.g. concussion), traumatic brain injury, stress, dehydration, nerve palsy (e.g., cranial or peripheral), hypoglycemia, migraine, multiple sclerosis, peripheral vascular disease, peripheral neuropathy, seizure (e.g., grand mal seizure), subdural hematoma, syncope, and transient unilateral weakness. Biomarkers of ischemic stroke disclosed herein can be those that can distinguish acute ischemic stroke from these stroke-mimicking conditions. In some cases, the biomarkers disclosed herein can identify a stroke mimicking condition disclosed herein. In some cases, the biomarkers disclosed herein can identify a non-stroke condition disclosed herein.

A biomarker can refer to a biomolecule. In some cases, a biomarker can be a biomolecule associated with a disease. When associated with a disease, a biomarker can have a profile different under the disease condition compared to a non-disease condition. Biomarkers can be any class of biomolecules, including polynucleotides, polypeptides, carbohydrates and lipids. In some cases, a biomarker can be a polynucleotide. In some cases, a biomarker can be a polypeptide. A polynucleotide can be any type of nucleic acid molecule, including DNA, RNA, a hybridization thereof, or any combination thereof. For example, a polynucleotide can be cDNA, genomic DNA, mRNA, tRNA, rRNA, or microRNA. In some cases, a polynucleotide can be a cell-free nucleic acid molecule circulating in blood or a cellular nucleic acid molecule in a cell circulating in blood. A polypeptide or protein can be contemplated to include any fragments thereof, in particular, immunologically detectable fragments. A biomarker can also include one or more fragments of the biomarker having sufficient sequence such that it still possesses the same or substantially the same function as the full-size biomarker. An active fragment of a biomarker retains 100% of the activity of the full-size biomarker, or at least about 99%, 95%, 90%, 85%, 80%, 75%, 70%, 65%, 60%, 55%, or at least 50% of its activity. In certain cases, an active fragment of a biomarker can be detectable (e.g., a polypeptide detectable by an antibody, or a polynucleotide detectable by an oligonucleotide). A biomarker of ischemic stroke can be a biomolecule associated with ischemic stroke. In some cases, a biomarker of ischemic stroke can be a biomolecule associated with ischemic stroke, but not associated with other diseases. In some cases, a biomarker of ischemic stroke can be a biomolecule associated with ischemic stroke and other diseases.

The methods, devices, and kits herein can be used to assess a condition. A condition can be a disease or a risk of a disease in a subject. For example, the methods can comprise measuring the expression of a group of biomarkers in a sample from a subject, and assessing a disease or a risk of a disease in a subject based on the expression. In some cases, a condition can be a risk factor for strokes, e.g., high blood pressure, atrial fibrillation, high cholesterol, diabetes, atherosclerosis, circulation problems, tobacco use, alcohol use, physical inactivity, obesity, age, gender, race, family history, previous stroke, previous transient ischemic attack (TIA), fibromuscular dysplasia, patent foramen ovale, or any combination thereof. If one or more risk factors are known in a subject, the risk factors can be used, e.g., in combination with the expression of a group of biomarkers, to assess ischemic stroke or a risk of ischemic stroke in the subject.

A condition can be a disease. A disease can be ischemic stroke. In some cases, a disease can be Alzheimer's disease or Parkinson's disease. In some cases, a disease can be an autoimmune disease such as acute disseminated encephalomyelitis (ADEM), acute necrotizing hemorrhagic leukoencephalitis, Addison's disease, agammaglobulinemia, allergic asthma, allergic rhinitis, alopecia areata, amyloidosis, ankylosing spondylitis, anti-GBM/anti-TBM nephritis, antiphospholipid syndrome (APS), autoimmune aplastic anemia, autoimmune dysautonomia, autoimmune hepatitis, autoimmune hyperlipidemia, autoimmune immunodeficiency, autoimmune inner ear disease (AIED), autoimmune myocarditis, autoimmune pancreatitis, autoimmune retinopathy, autoimmune thrombocytopenic purpura (ATP), autoimmune thyroid disease, axonal & neuronal neuropathies, Balo disease, Behcet's disease, bullous pemphigoid, cardiomyopathy, Castlemen disease, celiac sprue (non-tropical), Chagas disease, chronic fatigue syndrome, chronic inflammatory demyelinating polyneuropathy (CIDP), chronic recurrent multifocal ostomyelitis (CRMO), Churg-Strauss syndrome, cicatricial pemphigoid/benign mucosal pemphigoid, Crohn's disease, Cogan's syndrome, cold agglutinin disease, congenital heart block, coxsackie myocarditis, CREST disease, essential mixed cryoglobulinemia, demyelinating neuropathies, dermatomyositis, Devic's disease (neuromyelitis optica), discoid lupus, Dressler's syndrome, endometriosis, eosinophillic fasciitis, erythema nodosum, experimental allergic encephalomyelitis, Evan's syndrome, fibromyalgia, fibrosing alveolitis, giant cell arteritis (temporal arteritis), glomerulonephritis, Goodpasture's syndrome, Grave's disease, Guillain-Barre syndrome, Hashimoto's encephalitis, Hashimoto's thyroiditis, hemolytic anemia, Henock-Schoniein purpura, herpes gestationis, hypogammaglobulinemia, idiopathic thrombocytopenic purpura (ITP), IgA nephropathy, immunoregulatory lipoproteins, inclusion body myositis, insulin-dependent diabetes (type 1), interstitial cystitis, juvenile arthritis, juvenile diabetes, Kawasaki syndrome, Lambert-Eaton syndrome, leukocytoclastic vasculitis, lichen planus, lichen sclerosus, ligneous conjunctivitis, linear IgA disease (LAD), Lupus (SLE), Lyme disease, Meniere's disease, microscopic polyangitis, mixed connective tissue disease (MCTD), Mooren's ulcer, Mucha-Habermann disease, multiple sclerosis, myasthenia gravis, myositis, narcolepsy, neuromyelitis optica (Devic's), neutropenia, ocular cicatricial pemphigoid, optic neuritis, palindromic rheumatism, PANDAS (Pediatric Autoimmune Neuropsychiatric Disorders Associated with Streptococcus), paraneoplastic cerebellar degeneration, paroxysmal nocturnal hemoglobinuria (PNH), Parry Romberg syndrome, Parsonnage-Turner syndrome, pars plantis (peripheral uveitis), pemphigus, peripheral neuropathy, perivenous encephalomyelitis, pernicious anemia, POEMS syndrome, polyarteritis nodosa, type I, II & III autoimmune polyglandular syndromes, polymyalgia rheumatic, polymyositis, postmyocardial infarction syndrome, postpericardiotomy syndrome, progesterone dermatitis, primary biliary cirrhosis, primary sclerosing cholangitis, psoriasis, psoriatic arthritis, idiopathic pulmonary fibrosis, pyoderma gangrenosum, pure red cell aplasis, Raynaud's phenomena, reflex sympathetic dystrophy, Reiter's syndrome, relapsing polychondritis, restless legs syndrome, retroperitoneal fibrosis, rheumatic fever, rheumatoid arthritis, sarcoidosis, Schmidt syndrome, scleritis, scleroderma, Slogren's syndrome, sperm and testicular autoimmunity, stiff person syndrome, subacute bacterial endocarditis (SBE), sympathetic ophthalmia, Takayasu's arteritis, temporal arteritis/giant cell arteries, thrombocytopenic purpura (TPP), Tolosa-Hunt syndrome, transverse myelitis, ulcerative colitis, undifferentiated connective tissue disease (UCTD), uveitis, vasculitis, vesiculobullous dermatosis, vitiligo or Wegener's granulomatosis or, chronic active hepatitis, primary biliary cirrhosis, cadilated cardiomyopathy, myocarditis, autoimmune polyendocrine syndrome type I (APS-I), cystic fibrosis vasculitides, acquired hypoparathyroidism, coronary artery disease, pemphigus foliaceus, pemphigus vulgaris, Rasmussen encephalitis, autoimmune gastritis, insulin hypoglycemic syndrome (Hirata disease), Type B insulin resistance, acanthosis, systemic lupus erythematosus (SLE), pernicious anemia, treatment-resistant Lyme arthritis, polyneuropathy, demyelinating diseases, atopic dermatitis, autoimmune hypothyroidism, vitiligo, thyroid associated ophthalmopathy, autoimmune coeliac disease, ACTH deficiency, dermatomyositis, Sjogren syndrome, systemic sclerosis, progressive systemic sclerosis, morphea, primary antiphospholipid syndrome, chronic idiopathic urticaria, connective tissue syndromes, necrotizing and crescentic glomerulonephritis (NCGN), systemic vasculitis, Raynaud syndrome, chronic liver disease, visceral leishmaniasis, autoimmune C1 deficiency, membrane proliferative glomerulonephritis (MPGN), prolonged coagulation time, immunodeficiency, atherosclerosis, neuronopathy, paraneoplastic pemphigus, paraneoplastic stiff man syndrome, paraneoplastic encephalomyelitis, subacute autonomic neuropathy, cancer-associated retinopathy, paraneoplastic opsoclonus myoclonus ataxia, lower motor neuron syndrome and Lambert-Eaton myasthenic syndrome.

In some cases, a disease can be a cancer such as Acute lymphoblastic leukemia, Acute myeloid leukemia, Adrenocortical carcinoma, AIDS-related cancers, AIDS-related lymphoma, Anal cancer, Appendix cancer, Astrocytoma, childhood cerebellar or cerebral, Basal cell carcinoma, Bile duct cancer, extrahepatic, Bladder cancer, Bone cancer, Osteosarcoma/Malignant fibrous histiocytoma, Brainstem glioma, Brain tumor, Brain tumor, cerebellar astrocytoma, Brain tumor, cerebral astrocytoma/malignant glioma, Brain tumor, ependymoma, Brain tumor, medulloblastoma, Brain tumor, supratentorial primitive neuroectodermal tumors, Brain tumor, visual pathway and hypothalamic glioma, Breast cancer, Bronchial adenomas/carcinoids, Burkitt lymphoma, Carcinoid tumor, childhood, Carcinoid tumor, gastrointestinal, Carcinoma of unknown primary, Central nervous system lymphoma, primary, Cerebellar astrocytoma, childhood, Cerebral astrocytoma/Malignant glioma, childhood, Cervical cancer, Childhood cancers, Chronic lymphocytic leukemia, Chronic myelogenous leukemia, Chronic myeloproliferative disorders, Colon Cancer, Cutaneous T-cell lymphoma, Desmoplastic small round cell tumor, Endometrial cancer, Ependymoma, Esophageal cancer, Ewing's sarcoma in the Ewing family of tumors, Extracranial germ cell tumor, Childhood, Extragonadal Germ cell tumor, Extrahepatic bile duct cancer, Eye Cancer, Intraocular melanoma, Eye Cancer, Retinoblastoma, Gallbladder cancer, Gastric (Stomach) cancer, Gastrointestinal Carcinoid Tumor, Gastrointestinal stromal tumor (GIST), Germ cell tumor: extracranial, extragonadal, or ovarian, Gestational trophoblastic tumor, Glioma of the brain stem, Glioma, Childhood Cerebral Astrocytoma, Glioma, Childhood Visual Pathway and Hypothalamic, Gastric carcinoid, Hairy cell leukemia, Head and neck cancer, Heart cancer, Hepatocellular (liver) cancer, Hodgkin lymphoma, Hypopharyngeal cancer, Hypothalamic and visual pathway glioma, childhood, Intraocular Melanoma, Islet Cell Carcinoma (Endocrine Pancreas), Kaposi sarcoma, Kidney cancer (renal cell cancer), Laryngeal Cancer, Leukemias, Leukemia, acute lymphoblastic (also called acute lymphocytic leukemia), Leukemia, acute myeloid (also called acute myelogenous leukemia), Leukemia, chronic lymphocytic (also called chronic lymphocytic leukemia), Leukemia, chronic myelogenous (also called chronic myeloid leukemia), Leukemia, hairy cell, Lip and Oral Cavity Cancer, Liver Cancer (Primary), Lung Cancer, Non-Small Cell, Lung Cancer, Small Cell, Lymphomas, Lymphoma, AIDS-related, Lymphoma, Burkitt, Lymphoma, cutaneous T-Cell, Lymphoma, Hodgkin, Lymphomas, Non-Hodgkin (an old classification of all lymphomas except Hodgkin's), Lymphoma, Primary Central Nervous System, Marcus Whittle, Deadly Disease, Macroglobulinemia, Waldenström, Malignant Fibrous Histiocytoma of Bone/Osteosarcoma, Medulloblastoma, Childhood, Melanoma, Melanoma, Intraocular (Eye), Merkel Cell Carcinoma, Mesothelioma, Adult Malignant, Mesothelioma, Childhood, Metastatic Squamous Neck Cancer with Occult Primary, Mouth Cancer, Multiple Endocrine Neoplasia Syndrome, Childhood, Multiple Myeloma/Plasma Cell Neoplasm, Mycosis Fungoides, Myelodysplastic Syndromes, Myelodysplastic/Myeloproliferative Diseases, Myelogenous Leukemia, Chronic, Myeloid Leukemia, Adult Acute, Myeloid Leukemia, Childhood Acute, Myeloma, Multiple (Cancer of the Bone-Marrow), Myeloproliferative Disorders, Chronic, Nasal cavity and paranasal sinus cancer, Nasopharyngeal carcinoma, Neuroblastoma, Non-Hodgkin lymphoma, Non-small cell lung cancer, Oral Cancer, Oropharyngeal cancer, Osteosarcoma/malignant fibrous histiocytoma of bone, Ovarian cancer, Ovarian epithelial cancer (Surface epithelial-stromal tumor), Ovarian germ cell tumor, Ovarian low malignant potential tumor, Pancreatic cancer, Pancreatic cancer, islet cell, Paranasal sinus and nasal cavity cancer, Parathyroid cancer, Penile cancer, Pharyngeal cancer, Pheochromocytoma, Pineal astrocytoma, Pineal germinoma, Pineoblastoma and supratentorial primitive neuroectodermal tumors, childhood, Pituitary adenoma, Plasma cell neoplasia/Multiple myeloma, Pleuropulmonary blastoma, Primary central nervous system lymphoma, Prostate cancer, Rectal cancer, Renal cell carcinoma (kidney cancer), Renal pelvis and ureter, transitional cell cancer, Retinoblastoma, Rhabdomyosarcoma, childhood, Salivary gland cancer, Sarcoma, Ewing family of tumors, Sarcoma, Kaposi, Sarcoma, soft tissue, Sarcoma, uterine, Sézary syndrome, Skin cancer (nonmelanoma), Skin cancer (melanoma), Skin carcinoma, Merkel cell, Small cell lung cancer, Small intestine cancer, Soft tissue sarcoma, Squamous cell carcinoma—see Skin cancer (nonmelanoma), Squamous neck cancer with occult primary, metastatic, Stomach cancer, Supratentorial primitive neuroectodermal tumor, childhood, T-Cell lymphoma, cutaneous—see Mycosis Fungoides and Sézary syndrome, Testicular cancer, Throat cancer, Thymoma, childhood, Thymoma and Thymic carcinoma, Thyroid cancer, Thyroid cancer, childhood, Transitional cell cancer of the renal pelvis and ureter, Trophoblastic tumor, gestational, Unknown primary site, carcinoma of, adult, Unknown primary site, cancer of, childhood, Ureter and renal pelvis, transitional cell cancer, Urethral cancer, Uterine cancer, endometrial, Uterine sarcoma, Vaginal cancer, Visual pathway and hypothalamic glioma, childhood, Vulvar cancer, Waldenström macroglobulinemia, Wilms tumor (kidney cancer), childhood.

In some cases, a disease can be inflammatory disease, infectious disease, cardiovascular disease and metabolic disease. Specific infectious diseases include, but is not limited to AIDS, anthrax, botulism, brucellosis, chancroid, chlamydial infection, cholera, coccidioidomycosis, cryptosporidiosis, cyclosporiasis, dipheheria, ehrlichiosis, arboviral encephalitis, enterohemorrhagic Escherichia coli, giardiasis, gonorrhea, dengue fever, haemophilus influenza, Hansen's disease (Leprosy), hantavirus pulmonary syndrome, hemolytic uremic syndrome, hepatitis A, hepatitis B, hepatitis C, human immunodeficiency virus, legionellosis, listeriosis, lyme disease, malaria, measles. Meningococcal disease, mumps, pertussis (whooping cough), plague, paralytic poliomyelitis, psittacosis, Q fever, rabies, rocky mountain spotted fever, rubella, congenital rubella syndrome (SARS), shigellosis, smallpox, streptococcal disease (invasive group A), streptococcal toxic shock syndrome, streptococcus pneumonia, syphilis, tetanus, toxic shock syndrome, trichinosis, tuberculosis, tularemia, typhoid fever, vancomycin intermediate resistant staphylocossus aureus, varicella, yellow fever, variant Creutzfeldt-Jakob disease (vCJD), Eblola hemorrhagic fever, Echinococcosis, Hendra virus infection, human monkeypox, influenza A, H5N1, lassa fever, Margurg hemorrhagic fever, Nipah virus, O'nyong fever, Rift valley fever, Venezuelan equine encephalitis and West Nile virus.

In some embodiments, the methods, device and kits described herein can detect one or more of the diseases disclosed herein. In some embodiments, one or more of the biomarkers disclosed herein can be used to assess one or more disease disclosed herein. In some embodiments, one or more of the biomarkers disclosed herein can be used to detect one or more diseases disclosed herein.

The group of biomarkers disclosed herein can comprise one or more of an anthrax toxin receptor, a serine/threonine-protein kinase, a pyruvate dehydrogenase lipoamide kinase, a cluster of differentiation family member, myelin and lymphocyte protein (MAL), an inhibitor of Ras-ERK pathway, a member of inhibitor of DNA binding family, a lysosomal cysteine proteinase, a motor protein, and a receptor for pigment epithelium-derived factor. For example, the group of biomarkers disclosed herein can comprise one, two, three, four, five, six, seven, eight, nine or ten of an anthrax toxin receptor, a serine/threonine-protein kinase, a pyruvate dehydrogenase lipoamide kinase, a cluster of differentiation family member, MAL, an inhibitor of Ras-ERK pathway, a member of inhibitor of DNA binding family, a lysosomal cysteine proteinase, a motor protein, and a receptor for pigment epithelium-derived factor.

Among the biomarkers, an anthrax toxin receptor can include anthrax toxin receptor 1 (ANTXR1) and anthrax toxin receptor 2 (ANTXR2). In some cases, an anthrax toxin receptor can be ANTXR1. Among the biomarkers, a serine/threonine-protein kinase can include serine/threonine-protein kinase 3 (STK3) and serine/threonine-protein kinase 4(STK4). In some cases, a serine/threonine-protein kinase can be STK3. Among the biomarkers, a pyruvate dehydrogenase lipoamide kinase can include pyruvate dehydrogenase lipoamide kinase isoenzyme 1 (PDK1), pyruvate dehydrogenase lipoamide kinase isoenzyme 2 (PDK2), pyruvate dehydrogenase lipoamide kinase isoenzyme 3 (PDK3), and pyruvate dehydrogenase lipoamide kinase isoenzyme 4 (PDK4). In some cases, a pyruvate dehydrogenase lipoamide kinase can be PDK4. Among the biomarkers, a cluster of differentiation family member can be cluster of differentiation 163 (CD163). Among the biomarkers, an inhibitor of Ras-ERK pathway can include GRB2-related adaptor protein (GRAP) and GRB2-related adaptor protein 2 (GRAP2). In some cases, an inhibitor of Ras-ERK pathway can be GRAP. Among the biomarkers, a member of inhibitor of DNA binding family can include inhibitor of DNA binding 1 (ID1), inhibitor of DNA binding 2 (ID2), inhibitor of DNA binding 3 (ID3), and inhibitor of DNA binding 4 (ID4). In some cases, a member of inhibitor of DNA binding family can be ID3. Among the biomarkers, a lysosomal cysteine proteinase can be cathepsins (CTS), including CTSB, CTSC, CTSF, CTSH, CTSK, CTSL1, CTSL2, CTSO, CTSS, CTSW, and CTSZ. Other CTS can be used as biomarkers herein, including CTSA, CTSD, CTSE, and CTSG. In some cases, a lysosomal cysteine proteinase can be CTSZ. Among the biomarkers, a motor protein can include a kinesin-like protein, including kinesin-like protein 5A (KIF5A), kinesin-like protein 5B (KIF5B), kinesin-like protein 5C (KIF5C), kinesin-like protein 3A (KIF3A), kinesin-like protein 3B (KIF3B), kinesin-like protein 17 (KIF17), kinesin-like protein 1A (KIF1A), kinesin-like protein 1B (KIF1B), kinesin-like protein 1C (KIF1C), kinesin-like protein 13A (KIF13A), kinesin-like protein 13B (KIF13B), kinesin-like protein 16B (KIF16B), kinesin-like protein 4 (KIF4), and kinesin-like protein 21B (KIF21B). In some cases, a kinesin-like protein can be KIF1B. Among the biomarkers, a receptor for pigment epithelium-derived factor includes plexin domain-containing protein 1 (PLXDC1) and plexin domain-containing protein 2 (PLXDC2). In some cases, a receptor for pigment epithelium-derived factor can be PLXDC1. In some cases, the group of biomarkers disclosed herein can comprise one, two, three, four, five, six, seven, eight, nine or ten of ANTXR2, STK3, PDK4, CD163, MAL, GRAP, ID3, CTSZ, KIF1B, and PLXDC2.

The group of biomarkers disclosed herein can comprise any combination of the biomarkers disclosed herein. The group of biomarkers disclosed herein can comprise an anthrax toxin receptor. The group of biomarkers disclosed herein can comprise an anthrax toxin receptor and a serine/threonine-protein kinase. The group of biomarkers disclosed herein can comprise an anthrax toxin receptor, a serine/threonine-protein kinase, and a pyruvate dehydrogenase lipoamide kinase. The group of biomarkers disclosed herein can comprise an anthrax toxin receptor, a serine/threonine-protein kinase, a pyruvate dehydrogenase lipoamide kinase, and a cluster of differentiation family member. The group of biomarkers disclosed herein can comprise an anthrax toxin receptor, a serine/threonine-protein kinase, a pyruvate dehydrogenase lipoamide kinase, a cluster of differentiation family member, and myelin and lymphocyte protein. The group of biomarkers disclosed herein can comprise an anthrax toxin receptor, a serine/threonine-protein kinase, a pyruvate dehydrogenase lipoamide kinase, a cluster of differentiation family member, myelin and lymphocyte protein, and an inhibitor of Ras-ERK pathway. The group of biomarkers disclosed herein can comprise an anthrax toxin receptor, a serine/threonine-protein kinase, a pyruvate dehydrogenase lipoamide kinase, a cluster of differentiation family member, myelin and lymphocyte protein, an inhibitor of Ras-ERK pathway, and a member of inhibitor of DNA binding family. The group of biomarkers disclosed herein can comprise an anthrax toxin receptor, a serine/threonine-protein kinase, a pyruvate dehydrogenase lipoamide kinase, a cluster of differentiation family member, myelin and lymphocyte protein, an inhibitor of Ras-ERK pathway, a member of inhibitor of DNA binding family, and a lysosomal cysteine proteinase. The group of biomarkers disclosed herein can comprise an anthrax toxin receptor, a serine/threonine-protein kinase, a pyruvate dehydrogenase lipoamide kinase, a cluster of differentiation family member, myelin and lymphocyte protein, an inhibitor of Ras-ERK pathway, a member of inhibitor of DNA binding family, a lysosomal cysteine proteinase, and a motor protein. The group of biomarkers disclosed herein can comprise an anthrax toxin receptor, a serine/threonine-protein kinase, a pyruvate dehydrogenase lipoamide kinase, a cluster of differentiation family member, myelin and lymphocyte protein, an inhibitor of Ras-ERK pathway, a member of inhibitor of DNA binding family, a lysosomal cysteine proteinase, a motor protein, and a receptor for pigment epithelium-derived factor. In some cases, the group of biomarkers can comprise 1, 2, 3, 4, 5, 6, 7, 8, 9, or 10 of an anthrax toxin receptor, a serine/threonine-protein kinase, a pyruvate dehydrogenase lipoamide kinase, a cluster of differentiation family member, myelin and lymphocyte protein, an inhibitor of Ras-ERK pathway, a member of inhibitor of DNA binding family, a lysosomal cysteine proteinase, a motor protein, and a receptor for pigment epithelium-derived factor.

The group of biomarkers disclosed herein can comprise ANTXR2. The group of biomarkers disclosed herein can comprise ANTXR2 and STK3. The group of biomarkers disclosed herein can comprise ANTXR2, STK3, and PDK4. The group of biomarkers disclosed herein can comprise ANTXR2, STK3, PDK4, and CD163. The group of biomarkers disclosed herein can comprise ANTXR2, STK3, PDK4, CD163, and MAL. The group of biomarkers disclosed herein can comprise ANTXR2, STK3, PDK4, CD163, MAL, and GRAP. The group of biomarkers disclosed herein can comprise ANTXR2, STK3, PDK4, CD163, MAL, GRAP, and ID3. The group of biomarkers disclosed herein can comprise ANTXR2, STK3, PDK4, CD163, MAL, GRAP, ID3, and CTSZ. The group of biomarkers disclosed herein can comprise ANTXR2, STK3, PDK4, CD163, MAL, GRAP, ID3, CTSZ, and KIF1B. The group of biomarkers disclosed herein can comprise ANTXR2, STK3, PDK4, CD163, MAL, GRAP, ID3, CTSZ, KIF1B, and PLXDC2. In some cases, the group of biomarkers can comprise 1, 2, 3, 4, 5, 6, 7, 8, 9, or 10 of ANTXR2, STK3, PDK4, CD163, MAL, GRAP, ID3, CTSZ, KIF1B, and PLXDC2.

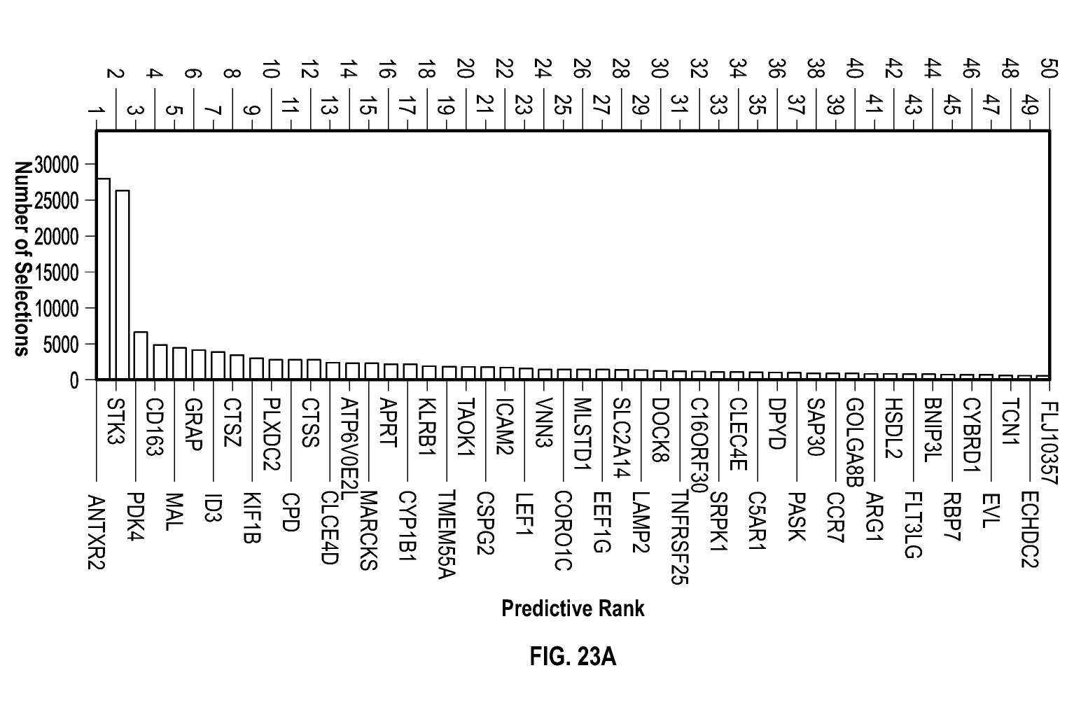

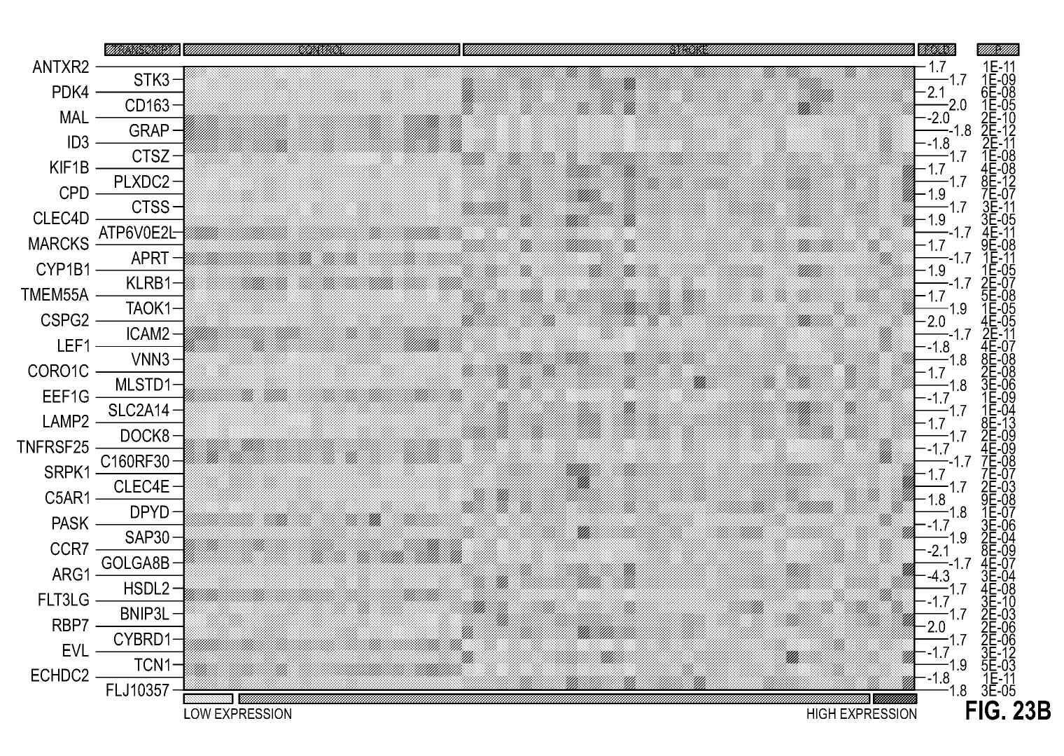

The group of biomarkers herein can comprise any number of biomarkers. For example, the group of biomarkers can comprise at least about 1, 2, 3, 4, 5, 6, 7, 8, 9, 10, 11, 12, 13, 14, 15, 16, 17, 18, 19, 20, 25, 30, 40, 50, 60, 70, 80, 90, 100, 200, 400, 800, or 1000 biomarkers. In some cases, the group of biomarkers comprises about 1, 2, 3, 4, 5, 6, 7, 8, 9, 10, 11, 12, 13, 14, 15, 16, 17, 18, 19, 20 biomarkers. In some cases, the group of biomarkers can comprise about 1, 2, 3, 4, 5, 6, 7, 8, 9, or 10 of ANTXR2, STK3, PDK4, CD163, MAL, GRAP, ID3, CTSZ, KIF1B, and PLXDC2. In some cases, the group of biomarkers can comprise at least about 1, 2, 3, 4, 5, 6, 7, 8, 9, 10, 15, 20, 30, 40, or 50 of the biomarkers shown in FIGS. 23A and 23B.

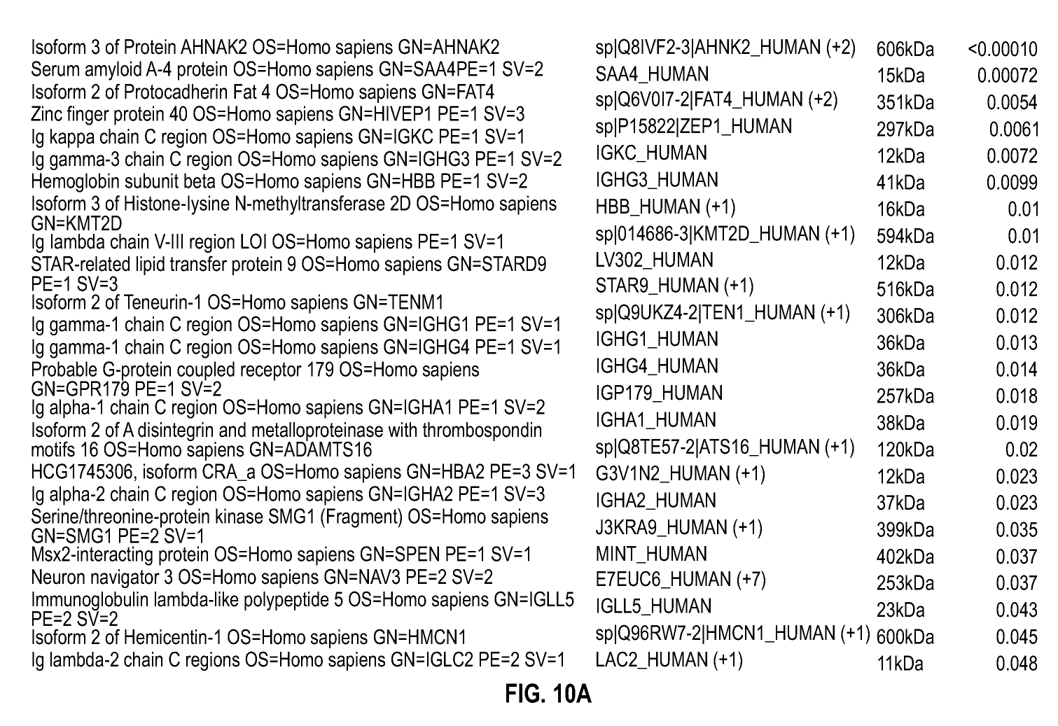

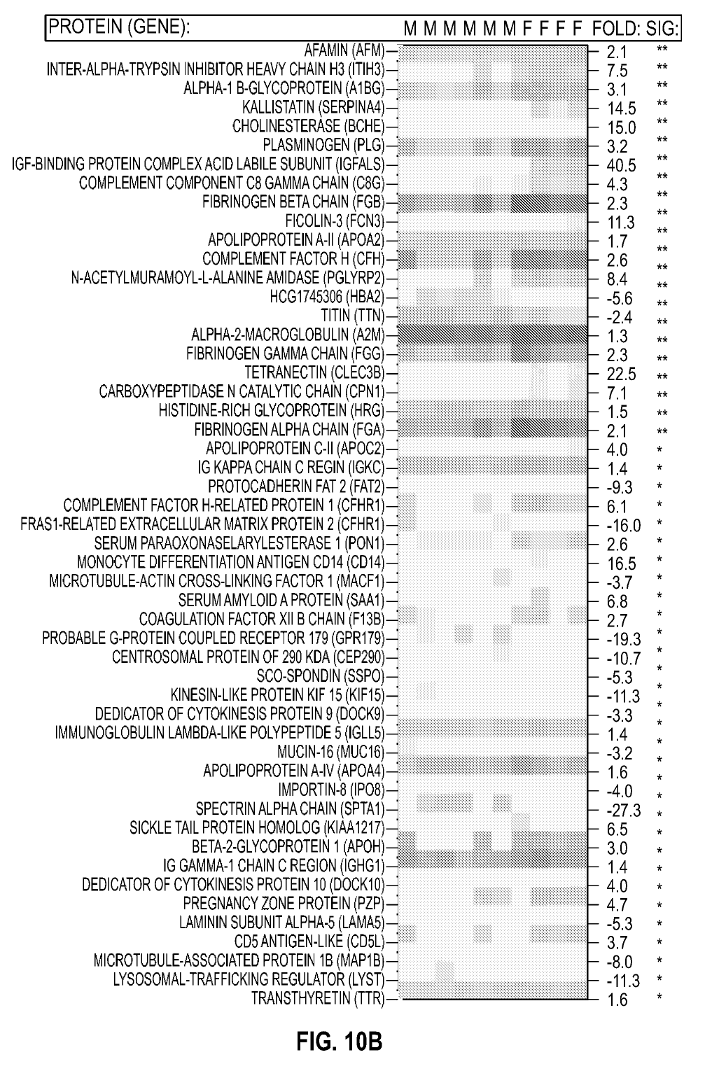

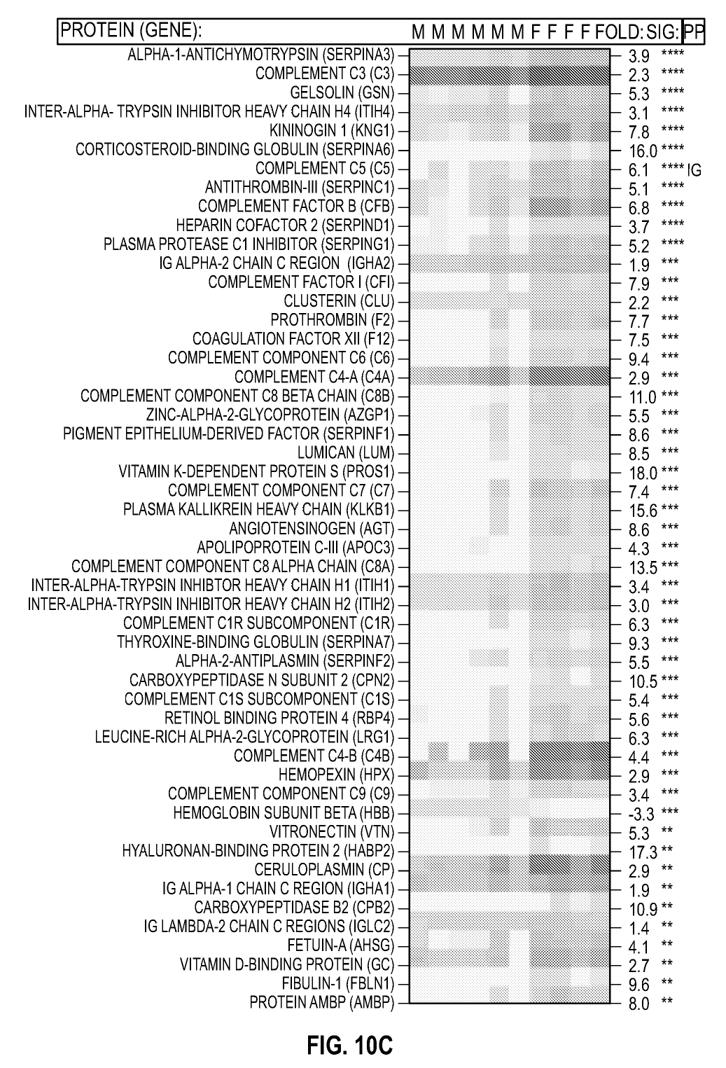

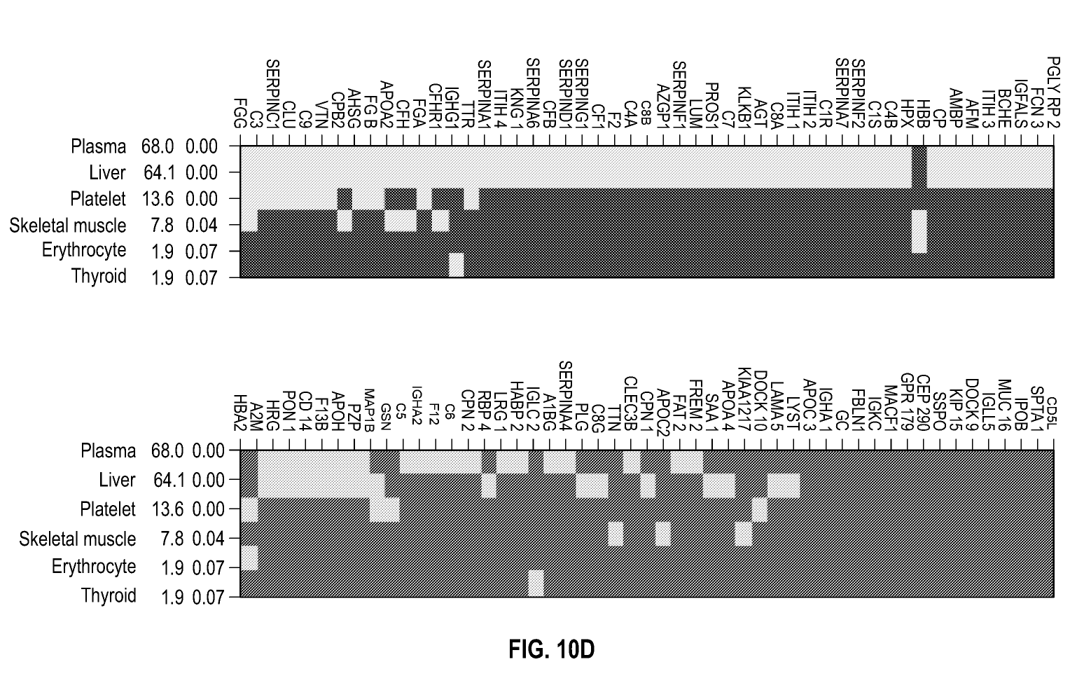

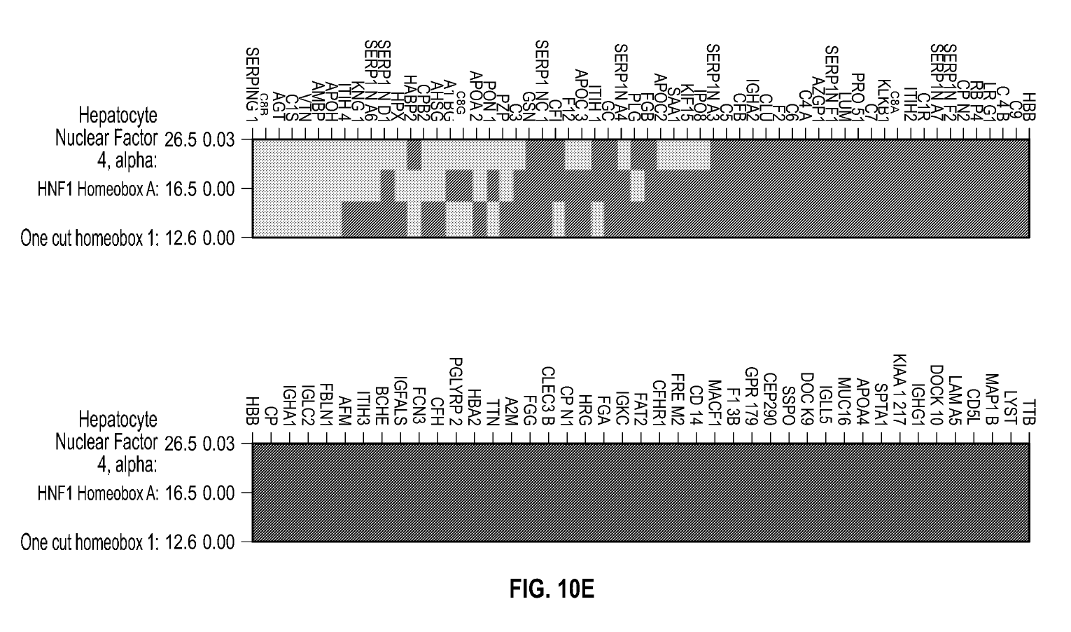

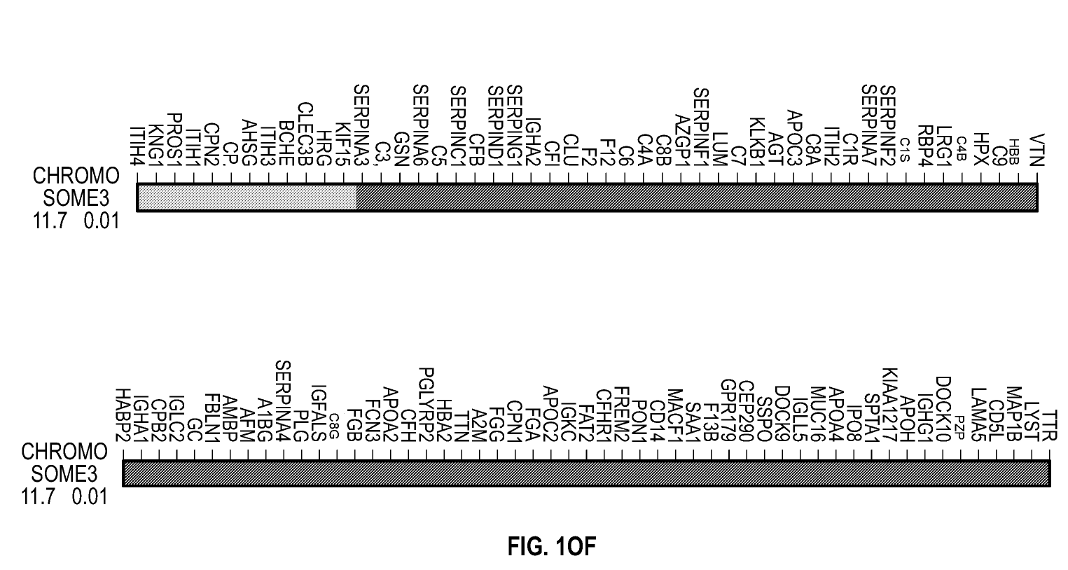

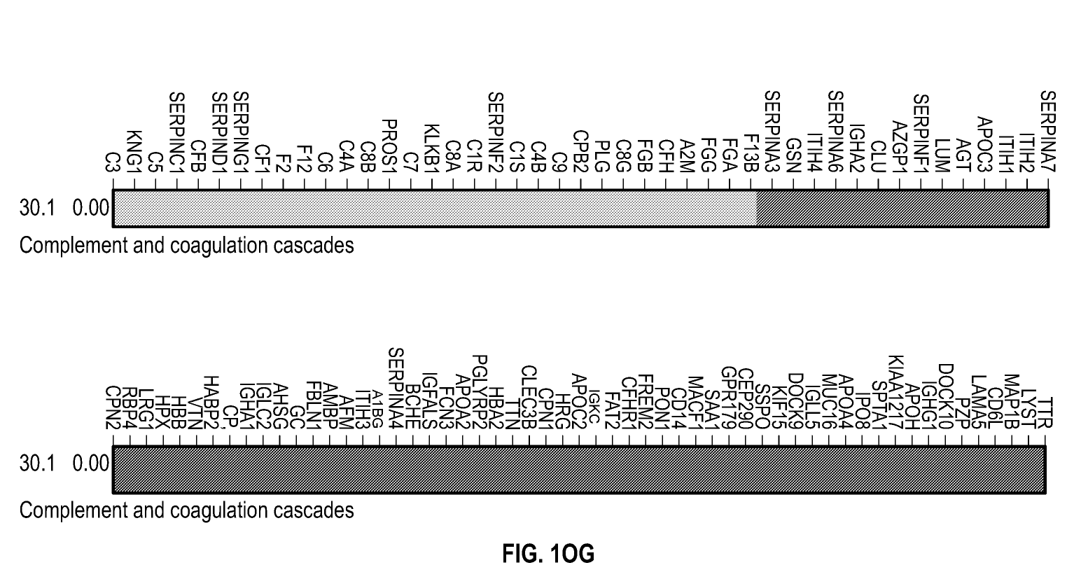

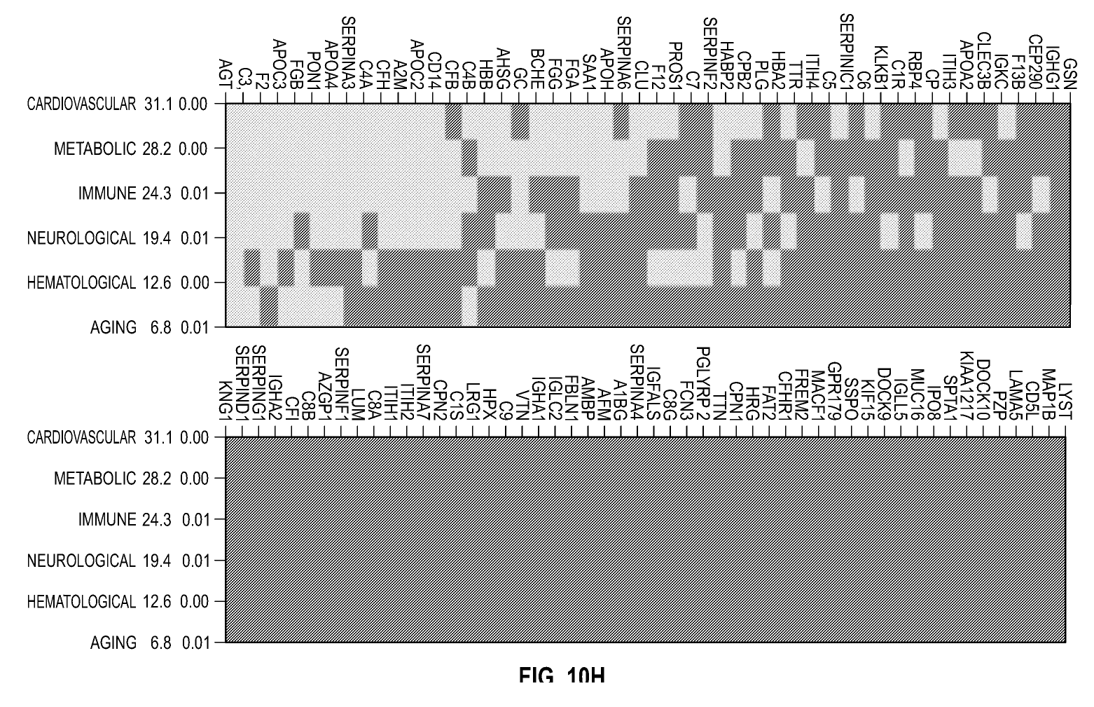

Biomarkers (e.g., biomarkers of ischemic stroke) disclosed herein can include at least one of Chemokine (C-C motif) ligand 19 (CCL19), Chemokine (C-C motif) ligand 21 (CCL21), Galectin 3, Receptor for advanced glycation end-products (RAGE), Epithelial neutrophil-activating protein 78 (ENA78), Granulocyte-macrophage colony-stimulating factor (GM-CSF), Cluster of differentiation 30 (CD30), chemokine receptor 7 (CCR7), chondroitin sulfate proteoglycan 2 (CSPG2), IQ motif-containing GTPase activation protein 1 (IQGAP1), orosomucoid 1 (ORM1), arginase 1 (ARG1), lymphocyte antigen 96 (LY96), matrix metalloproteinase 9 (MMP9), carbonic anhydrase 4 (CA4), s100 calcium binding proteinA12 (s100A12), or an active fragment thereof. In some cases, biomarkers (e.g., biomarkers of ischemic stroke) disclosed herein can include at least one polynucleotide encoding CCL19, CCL21, Galectin 3, RAGE, ENA78, GMCSF, CD30, CCR7, CSPG2, IQGAP1, ORM1, ARG1, LY96, MMP9, CA4, s100A12, Nav3, SAA, IGα, IGγ, IGκ, IGλ, or an active fragment thereof. Biomarkers (e.g., biomarkers of ischemic stroke) disclosed herein can include at least one cytokine or polynucleotide encoding thereof. In some cases, biomarkers (e.g., biomarkers of ischemic stroke) disclosed herein can include at least one of BAFF, MMP9, APP, Aggrecan, Galectin 3, Fas, RAGE, Ephrin A2, CD30, TNR1, CD27, CD40, TNFα, IL6, IL8, IL10, IL1β, IFNγ, RANTES, IL1α, IL4, IL17, IL2, GMCSF, ENA78, IL5, IL12P70, TARC, GroAlpha, IL33, BLCBCA, IL31, MCP2, IGG3, IGG4, Isoform 2 of Teneurin 1, and isoform 2 of aDisintegrin or an active fragment thereof. In some cases, biomarkers (e.g., biomarkers of ischemic stroke) disclosed herein can include at least one polynucleotide encoding BAFF, MMP9, APP, Aggrecan, Galectin 3, Fas, RAGE, Ephrin A2, CD30, TNR1, CD27, CD40, TNFα, IL6, IL8, IL10, IL1β, IFNγ, RANTES, IL1α, IL4, IL17, IL2, GMCSF, ENA78, IL5, IL12P70, TARC, GroAlpha, IL33, BLCBCA, IL31, MCP2, TLR2, TLR4, JAK2, CCR7, AKAP7, IL10, SYK, IL8, MyD88, CD3, CD4, IL22R, IL22, CEBPB, polypeptides listed in FIGS. 10A-10H or an active fragment thereof.

The amino acid and corresponding nucleic acid sequences of the biomarkers of the invention are known in the art and can be found in publicly available publications and databases. Exemplary sequences are set forth in Table 1 in the form of GenBank accession numbers.

TABLE 1
Exemplary biomarkers and accession numbers

| | Accession No. | Accession No. |
|---|---|---|
| Gene name | (mRNA) | (protein) |
| Chemokine (C-C motif) | NM_001838.2 | NP 001829 |
| receptor 7 (CCR7) | | |
| Versican (VCAN) | NM_004385.2 | NP 004376 |
| (CSPG2) | | |
| IQ motif containing | NM_003870.3 | NP 003861.1 |
| GTPase activating | | |
| protein 1 (IQGAP 1) | | |
| Orosomucoid 1 (ORM 1) | NM_000607.2 | NP 000598.2 |
| Arginase, liver (ARG 1) | NM_000045.2 | NP 000036.2 |
| Lymphocyte antigen 96 | NM_015364.3 | NP 056179.2 |
| (L Y96) | | |
| Matrix metallopeptidase | NM_004994.2 | NP 004985.2 |
| 9 (gelatinase B, 92 kDa | | |
| gelatinase, 92 kDa type | | |
| IV collagenase) (MMP9) | | |
| Carbonic anhydrase IV | NM 000717.3 | NP 000708.1 |
| (CA4) | | |
| S100 calcium binding | NM_005621.1 | NP 005612.1 |
| protein A12 (S100A12) | | |
| B-cell activating factor | AF116456 | AAD25356 |
| (BAFF) | | |
| Amyloid precursor | NM_000484 | BAA22264 |
| protein (APP) (transcript | | |
| variant 1) | | |
| Aggrecan (transcript | NM_001135 | AAH36445 |
| variant 1) | | |
| Galectin-3 | AB006780 | BAA22164 |
| Fas (isoform 1 precursor) | NM_007987.2 | AAH12479 |
| Receptor for Advanced | NM_001136.4 | AAH26069 |
| Glycation Endproducts | | |
| (RAGE) (isoform 1 | | |
| precursor) | | |
| Ephrin-A2 | NM_001405.3 | EAW69517 |
| CD30 (isoform 1 | NM_001243.4 | CAC16652 |
| precursor) | | |
| TNFR1 | NM_001065.3 | AAA61201 |
| CD27 | NM_001242.4 | NP_001233.1 |
| CD40 (isoform 1) | NM_001250.5 | AAH64518 |
| TNFα | NM_000594.3 | NP_000585.2 |
| IL-6 | NM_000600.3 | NP_000591.1 |
| IL-8 | NM_000584.3 | NM_000584.3 |
| IL-10 | XM_011509506.1 | XP_011507808.1 |
| IL-Iβ | NM_000576.2 | NP_000567.1 |
| IFNγ | NM_000619.2 | NP_000610.2 |
| Regulated on activation, | NM_002985.2 | EAW80120 |
| normal T cell expressed | | |
| and secreted (RANTES) | | |
| (isoform 1) | | |
| IL-1α | NM_000575.3 | NP_000566.3 |
| IL-4 (isoform 1) | NM_000589.3 | AAH70123 |
| IL-17 | NM_002190.2 | AAC50341 |
| IL-2 | NM_000586.3 | AAB46883 |
| Granulocyte-macrophage | NM_000758.3 | AAA98768 |
| colony-stimulating factor | | |
| (GMCSF) | | |
| Epithelial-derived | NM_002994.4 | CAG33709 |
| neutrophil-activating | | |
| peptide 78 (ENA-78) | | |
| IL-5 | NM_000879.2 | AAA98620.1 |
| IL12/IL-23 p70 | NM_002187.2 | NP_002178.2 |
| Thymus and activation- | NM_002987.2 | EAW82921.1 |
| regulated chemokine | | |
| (TARC) | | |
| GRO-alpha | NM_001511. | AAH11976.1 |
| IL-33 (isoform 3) | NM_033439.3 | AAH47085.1 |
| CXCL13 (BLCBCA) | NM_006419.2 | AAH12589.1 |
| IL-31 | XM_011538326.1 | EAW98310 |
| Monocyte chemotactic | NM_005623.2 | CAA71760.1 |
| protein 2 (MCP-2) | | |

A biomarker can exist in multiple forms, each of which is encompassed herein. For example, variants of a biomarker herein can exist in which a small number, e.g., 1, 2, 3, 4, 5, 6, 7, 8, 9, 10 or more, of nucleotides or amino acid residues are different in relation to the exemplary accession numbers set forth in Table 1. However, these variants are intended to be used in the methods, kits and devices herein. In addition, a biomarker herein can also include the “derivatives” of the biomarker. A “derivative” of a biomarker (or of its encoding nucleic acid molecule) to a modified form of the biomarker. A modified form of a given biomarker can include at least one amino acid substitution, deletion, insertion or combination thereof, wherein said modified form retains a biological activity of an unmodified form. An amino acid substitution can be considered “conservative” when the substitution results in similar structural or chemical properties (e.g., replacement of leucine with isoleucine). An amino acid substitution can be “non-conservative” in nature wherein the structure and chemical properties vary (e.g., replacement of arginine with alanine). A modified form of a given biomarker can include chemical modifications, wherein a modified form retains a biological activity of a given biomarker. Such modifications include, but are not limited to, glycosylation, phosphorylation, acetylation, alkylation, methylation, biotinylation, glutamylation glycylation, isoprenylation, lipoylation, pegylation, phosphopantetheinylation, sulfation, selenation, and C-terminal amidation. Other modifications include those involving other proteins such as ISGylation, SUMOylation, and ubiquitination. In addition, modifications can also include those involved in changing the chemical nature of an amino acid such as deimination and deamidation.

Biomarkers herein can include biomarkers that pertain to other diseases or conditions other than ischemic stroke, including any other type of stroke, or other non-stroke conditions, in the event a user wishes to test or detect not only ischemic stroke, but also other conditions at the same time or using the same panel or set of biomarkers. Non-limiting examples of other such biomarkers include those related to blood pressure (e.g., A-type natriuretic peptide, C-type antriuretic peptide, urotensin II, vasopressen, calcitonin, angiotensin II, adrenomedullin, and endothenlins), coagulation and hemostasis (e.g., D-dimer, plasmin, b-thromboglobulin, platelet factor 4, fibrinopeptide A, platelet-derived growth factor, prothrombin, P-selectin and thrombin), acute phase response (e.g., C-reactive protein, mannose-binding protein, human neutrophil elastase, inducible nitric oxide synthase, lysophosphatidic acid, malondialdehyde LDL, lipopolysaccharide binding protein) and biomarkers related to inflammation (e.g., interleukins, tumor necrosis factor, myeloperoxidase, soluble intercellular adhesion molecule, vascular cell adhesion molecule, monocyte chemotactic protein-1). Such other biomarkers can assist in gaining a better overall clinical picture of the health of a patient and the potential causes of stroke. Such biomarkers can be selected on the basis of the knowledge of one of ordinary skill in the art. Additional examples of such biomarkers can be found in the art, for example, in U.S. Pat. No. 7,608,406, which is incorporated herein by reference in its entirety.

Methods for identifying one or more biomarkers of ischemic stroke can comprise measuring a profile of polynucleotides in a first ischemic stroke sample, and measuring a profile of polypeptides in a second ischemic stroke sample. In some cases, the first and second ischemic stroke samples can be from the same subject (e.g., the same ischemic stroke patient). In some cases, the first and second ischemic stroke samples can be from different subjects. The first and second ischemic stroke samples can be different aliquots of a single sample. For example, the first and second ischemic stroke samples can be different aliquots of the same blood sample from an ischemic stroke subject. In some cases, the first and second ischemic stroke samples can be from different samples (e.g., blood samples drawn from different subjects or from the same subject but at different times). In some cases, the first and second ischemic stroke samples can be different types of samples. For example, one ischemic stroke sample can be a blood sample and the other ischemic stroke sample can be a solid tissue sample. In another example, one ischemic stroke sample can be plasma and the other ischemic stroke sample can be blood cells.

A profile of polynucleotides can include the characteristics and/or the quantities of the polynucleotides. A profile of polynucleotides can include the expression levels, epigenetic modifications, and/or genetic variations of one or more polynucleotides in a sample of a subject. In some cases, the expression levels of one or more polynucleotides can be the mRNA level of one or more genes. For example, a profile of polynucleotides can be mRNA level of one or more genes in a whole blood sample of a patient. The epigenetic modifications of one or more polynucleotides can include acetylation, methylation, ubiquitylation, phosphorylation, sumoylation, ribosylation, or citrullination of one or more polynucleotides or active fragments thereof. For example, a profile of polynucleotides can be the methylation level of one or more polynucleotides in a sample. Genetic variations of one or more polynucleotides can include single nucleotide variations (SNV), insertions, deletions, insertion/deletions, rearrangements, copy number variations (CNV) of one or more genes or fragments thereof. For example, a profile of polynucleotides can be the level of genes that carry one or more deletions in a sample. A profile of polynucleotides can also include polymorphism (e.g., single nucleotides polymorphism (SNP)) of one or more genes in a sample. In some cases, a profile of polynucleotides can be the expression level of any types of nucleic acids. For example, a profile of polynucleotides can be the level of miRNA expressed from the genome. In some cases, a profile of polynucleotides can also include the concentration of cell-free polynucleotides in a bodily fluid (e.g., blood). For example, a profile of polynucleotides can be the level of cell-free DNA of one or more genomic DNA fragments in blood. In another example, a profile of polynucleotides can be the level of one or more species of microRNA circulating in blood.

In some cases, a profile of polynucleotides can comprise an expression pattern of the polynucleotides. For example, an expression pattern of the polynucleotides can be the expression level of the polynucleotides. In another example, an expression pattern of the polynucleotides can be the expression level differences of the polynucleotides compared to a polynucleotides reference profile.

A profile of polynucleotides can be measured by a nucleic acid analysis method. In some cases, a nucleic acid analysis method can be a polymerase chain reaction (PCR). Examples of PCR include amplified fragment length polymorphism PCR, allele-specific PCR, Alu PCR, asymmetric PCR, colony PCR, helicase dependent PCR, hot start PCR, inverse PCR, in situ PCR, intersequence-specific PCR, digital PCR, droplet digital PCR, linear-after-the-exponential-PCR (Late PCR), long PCR, nested PCR, duplex PCR, multiplex PCR, quantitative PCR, or single cell PCR. In a particular example, the nucleic acid analysis method can be quantitative PCR. In some cases, quantitative PCR can be real-time PCR, e.g., real-time quantitative PCR. In real-time quantitative PCR, the accumulation of amplification product can be measured continuously in both standard dilutions of target DNA and samples containing unknown amounts of target DNA. A standard curve can be constructed by correlating initial template concentration in the standard samples with the number of PCR cycles (Ct) necessary to produce a specific threshold concentration of product. In the test samples, target PCR product accumulation can be measured after the same Ct, which allows interpolation of target DNA concentration from the standard curve. In some cases, quantitative PCR can be competitive quantitative PCR. In competitive quantitative PCR, an internal competitor DNA can be added at a known concentration to both serially diluted standard samples and unknown (environmental) samples. After co-amplification, ratios of the internal competitor and target PCR products can be calculated for both standard dilutions and unknown samples, and a standard curve can be constructed that plots competitor-target PCR product ratios against the initial target DNA concentration of the standard dilutions. Given equal amplification efficiency of competitor and target DNA, the concentration of the latter in environmental samples can be extrapolated from this standard curve. In some cases, quantitative PCR can be relative quantitative PCR. Relative quantitative PCR can determine the relative concentrations of specific nucleic acids. For example, reverse transcriptase PCR can be performed on mRNA species isolated from a subject. By determining that the concentration of a specific mRNA species varies, the method can determine whether the gene encoding the specific mRNA species is differentially expressed. Quantitative PCR can be used to measure level of DNA or RNA in a sample. In some cases, a profile of polynucleotides can be measured using a microarray. For example, a profile of polynucleotides can be measured by a genomic scan using a genomic microarray.

The nucleic acid analysis method can also include a sequencing step. A sequencing step can be used to identify and/or quantify the polynucleotides analyzed by other methods herein. Sequencing can be performed by basic sequencing methods, including Maxam-Gilbert sequencing, chain-termination sequencing, shotgun sequencing or Bridge PCR. Sequencing can also be performed by massively parallel sequencing methods, including high-throughput sequencing, pyro-sequencing, sequencing-by-synthesis, single-molecule sequencing, nanopore sequencing, semiconductor sequencing, sequencing-by-ligation, sequencing-by-hybridization, RNA-Seq (Illumina), Digital Gene Expression (Helicos), Next generation sequencing, Single Molecule Sequencing by Synthesis (SMSS)(Helicos), massively-parallel sequencing, Clonal Single Molecule Array (Solexa), shotgun sequencing, Maxam-Gilbert or Sanger sequencing, primer walking, sequencing using Illumina, PacBio, SOLiD, Ion Torrent, 454, or nanopore platforms.

The expression of a group of biomarkers in a sample can be measured by contacting a panel of probes with the sample, where the probes bind to one or more biomarkers of the group of biomarkers. In some cases, one probe can bind to multiple biomarkers in the group of biomarkers. In some cases, one probe can specifically bind to only one particular biomarker in the group of biomarkers. In some cases, the panel of probes can bind to all biomarkers in the group of biomarkers. In some cases, the panel of probes can bind some, but not all, of the biomarkers in the group of biomarkers. In some cases, the panel of probes can bind to molecules derived from the biomarkers. For example, the probes can bind to DNA derived (e.g., reversely transcribed) from the RNA (e.g., mRNA or miRNA) of the biomarkers.

The expression of a group of biomarkers can be measured using an assay. The assay can be any nucleic acid analysis method or polypeptide analysis method disclosed herein. In some cases, the assay can be a combination of any nucleic acid method and polypeptide analysis method disclosed herein. The assay can be PCR, an immunoassay, or a combination thereof. The assay can be any type of PCR used in nucleic acid analysis disclosed herein. For example, the PCR can be a quantitative reverse transcription polymerase chain reaction. The assay can be an immunoassay. Examples of immunoassays include immunoprecipitation, particle immunoassays, immunonephelometry, radioimmunoassays, enzyme immunoassays (e.g., ELISA), fluorescent immunoassays, chemiluminescent immunoassays, and Western blot analysis.

A profile of polypeptides can include the characteristics and/or the quantities of the polypeptides. In some cases, a profile of polypeptides can be the expression level of the polypeptides. The expression level of polypeptides can be the concentration or absolute quantity of the polypeptides. In some cases, a profile of polypeptides can be the level of post-translational modification of the polypeptides. Polypeptides or proteins can exist in a plurality of different forms. These forms can result from either or both of pre- and post-translational modification. Pre-translationally modified forms include allelic variants, splice variants and RNA editing forms. Post-translationally modified forms include forms resulting from proteolytic cleavage (e.g., cleavage of a signal sequence or fragments of a parent protein), glycosylation, phosphorylation, lipidation, oxidation, methylation, cysteinylation, sulphonation and acetylation. The Post-translational modification of the polypeptides can include phosphorylation, acetylation, amination, methylation, glycosylation, lipidation, or any other chemical modifications of the polypeptides.

In some cases, a profile of polypeptides can comprise an expression pattern of the polypeptides. For example, an expression pattern of the polypeptides can be the expression level of the polypeptides. In another example, an expression pattern of the polypeptides can be the expression level differences of the polypeptides compared to a polypeptide reference profile. In some cases, an expression pattern can be an increase/decrease in expression of one or more biomarkers in a first group of biomarker in a disease condition. In some cases, an expression pattern can be an increase/decrease in expression of one or more biomarkers in a first group of biomarkers in a non-disease condition. In some cases, an expression pattern can be an increase/decrease in expression of one or more biomarkers in a second group of biomarker in a disease condition. In some cases, an expression pattern can be an increase/decrease in expression of one or more biomarkers in a second group of biomarker in a non-disease condition. In some cases, the expression pattern can be the level of CK-MB, a hematocrit percent, a prothrombin time, a white blood cell count, a lymphocyte count, a platelet count or a neutrophil percent in a disease and/or non-disease condition. In some cases, the expression pattern can be at least 1 biomarker is increased and/or at least 1 biomarker is decreased in a sample. In some cases at least about 1, 2, 3, 4, 5, 6, 7, 8, 9, 10, 15, 20, 30, 40, 50, 75, 100, 200, 500, 1000 biomarkers are increased in a sample. In some cases at least about 1, 2, 3, 4, 5, 6, 7, 8, 9, 10, 15, 20, 30, 40, 50, 75, 100, 200, 500, 1000 are decreased in a sample.

Expression patterns of biomarkers can be determined by statistical analysis. In some cases, an expression pattern of biomarkers can be measured by statistical regression. In another example, an expression pattern of biomarkers can be a multiple score of a first biomarker expression and a second biomarker expression. For example, the multiple score of biomarker 1×biomarker 2. In another example, an expression pattern of biomarkers can be a multiple score of a first biomarker expression and a second biomarker expression, wherein the first and second biomarkers are in the same or different treatment group and/or disease group. In another example, an expression pattern of biomarkers can be a ratio of a first biomarker expression to a second biomarker expression. In another example, an expression pattern of biomarkers can be a ratio of a first biomarker expression to a second biomarker expression, wherein the first and second biomarkers are in the same or different treatment group and/or disease group. In some aspects, the ratio of a first biomarker expression to a second biomarker expression can be in a range from about 0.01 to about 10000. In some aspects, the ratio of a first biomarker expression to a second biomarker expression can be at least about 0.5, 1, 1.5, 2, 3, 4, 5, 6, 7, 8, 9, 10, 20, 30, 40, 50, 60, 70, 80, 90, 100, 200, 500, or at least 1000. In another example, an expression pattern of biomarkers can be determined by multivariate statistical analysis. The multivariate statistical analysis may be principal component analysis, discriminant analysis, principal component analysis with discriminant analysis, partial least squares, partial least squares with discriminant analysis, canonical correlation, kernel principal component analysis, non-linear principal component analysis, factor analysis, multidimensional scaling, and cluster analysis. In another example, an expression pattern of biomarkers can be determined by principal components analysis. In another example, an expression pattern of biomarkers can be determined by machine learning and or pattern recognition.

A profile of polypeptides can be measured by a polypeptide analysis method. A polypeptide analysis method can include mass spectrometry, a multiplex assay, a microarray, an enzyme-linked immunosorbent assay (ELISA), or any combination thereof. Mass spectrometry (MS) can be used to resolve different forms of a protein because the different forms typically have different masses that can be resolved by mass spectrometry. Accordingly, if one form of a polypeptide or protein is a better biomarker for a disease than another form of the biomarker, mass spectrometry can be used to specifically detect and measure the useful form. MS can include time-of-flight (TOF) MS (e.g., Matrix-assisted laser desorption/ionization (MALDI) TOF MS), surface-enhanced laser desorption/ionization (MELDI) MS, electrospray ionization MS, or Fourier transform ion cyclotron resonance (FT-ICR) MS. A multiplex assay can include a phage display, an antibody profiling, or an assay using a Luminex platform. A microarray for analyzing a profile of polypeptides can include analytical microarrays, functional protein microarrays, or reverse phase protein microarrays. In some cases, a profile of polypeptides or proteins can be measured by a proteomic scan (e.g. a whole proteomic scan) using a proteomic microarray.

The ability of an analysis method to differentiate between different forms of a protein biomarker can depend upon the nature of the differences and the method used to measure. For example, an immunoassay using a monoclonal antibody can detect all forms of a protein containing the epitope and will not distinguish between them. However, a sandwich immunoassay that uses two antibodies directed against different epitopes on a protein can detect all forms of the protein that contain both epitopes and will not detect those forms that contain only one of the epitopes. One methodology for measuring a profile of biomarkers can combine mass spectrometry with immunoassay. First, a biospecific capture reagent (e.g., an antibody that recognizes the biomarker and other forms of it) can be used to capture the biomarker of interest. The biospecific capture reagent can be bound to a solid phase, such as a bead, a plate, a membrane or an array. After unbound materials are washed away, the captured analytes can be detected and/or measured by mass spectrometry. This method can also result in the capture of protein binding partners that are bound to the proteins or that are otherwise recognized by antibodies and that, themselves, can be biomarkers. Various forms of mass spectrometry are useful for detecting protein forms, including laser desorption approaches, such as traditional MALDI or SELDI, and electrospray ionization. The use of immobilized antibodies specific for biomarkers is also contemplated. The antibodies could be immobilized onto a variety of solid supports, such as magnetic or chromatographic matrix particles, the surface of an assay place (such as microtiter wells), pieces of a solid substrate material or membrane (such as plastic, nylon, paper), and the like. An assay strip could be prepared by coating the antibody or a plurality of antibodies in an array on solid support. This strip could then be dipped into the test sample and then processed quickly through washes and detection steps to generate a measurable signal, such as a colored spot.

The presence or level of a biomarker can be measured using any suitable immunoassay, for example, enzyme-linked immunoassays (ELISA), radioimmunoassays (RIAs), competitive binding assays, and the like. Specific immunological binding of an antibody to the biomarker can be detected directly or indirectly. Direct labels include fluorescent or luminescent tags, metals, dyes, radionuclides, and the like, attached to the antibody. Indirect labels include various enzymes well known in the art, such as alkaline phosphatase, horseradish peroxidase and the like.

The analysis of a plurality of biomarkers can be carried out separately or simultaneously with one test sample. For separate or sequential assay of biomarkers, suitable apparatuses can include clinical laboratory analyzers such as the ELECSYS® (Roche), the AXSYM® (Abbott), the ACCESS® (Beckman), the ADVIA® CENTAUR® (Bayer) immunoassay systems, the NICHOLS ADVANTAGE® (Nichols Institute) immunoassay system, etc. Apparatuses or protein chips can perform simultaneous assays of a plurality of biomarkers on a single surface. Useful physical formats comprise surfaces having a plurality of discrete, addressable locations for the detection of a plurality of different analytes. Such formats can include protein microarrays, or “protein chips” (see, e.g., Ng and Ilag, J. Cell Mol. Med. 6: 329-340 (2002)) and certain capillary devices (see e.g., U.S. Pat. No. 6,019,944). In these embodiments each discrete surface location can comprise antibodies to immobilize one or more analyte(s) (e.g., a biomarker) for detection at each location. Surfaces can alternatively comprise one or more discrete particles (e.g., microparticles or nanoparticles) immobilized at discrete locations of a surface, where the microparticles comprise antibodies to immobilize one analyte (e.g., a biomarker) for detection. The protein biochips can further include, for example, protein biochips produced by Ciphergen Biosystems, Inc. (Fremont, Calif.), Packard BioScience Company (Meriden Conn.), Zyomyx (Hayward, Calif.), Phylos (Lexington, Mass.) and Biacore (Uppsala, Sweden). Examples of such protein biochips are described in the following patents or published patent applications: U.S. Pat. No. 6,225,047; PCT International Publication No. WO 99/51773; U.S. Pat. No. 6,329,209, PCT International Publication No. WO 00/56934 and U.S. Pat. No. 5,242,828, each of which is incorporated by reference herein in its entirety.

Identifying biomarkers of ischemic stroke can comprise analyzing a profile of polynucleotides from an ischemic stroke sample. Analyzing a profile of polynucleotides can comprise comparing the profile of polynucleotides to a polynucleotides reference profile. In some cases, comparing a profile of polynucleotides to a reference profile can comprise determining expression level differences between the polynucleotides in the ischemic stroke sample and the polynucleotides in the reference profile. When the expression level of a polynucleotide in the ischemic stroke sample is up-regulated or down-regulated compared to the expression level of the polynucleotide in a reference profile, the polynucleotide can be identified as a biomarker. The biomarker can be associated with ischemic stroke. In some cases, further analysis can be carried out to identify the biomarker as a biomarker of ischemic stroke. A polynucleotide can be identified as a biomarker when an expression level difference in the polynucleotide of at least 0.5, 1, 1.5, 2, 3, 4, 5, 6, 7, 8, 9, 10, 20, 30, 40, 50, 60, 70, 80, 90, or at least 100 fold is detected in an ischemic stroke sample when compared to a polynucleotide reference profile. In some cases, a polynucleotide can be identified as a biomarker when an expression level difference in the polynucleotide increases at least 0.5, 1, 1.5, 2, 3, 4, 5, 6, 7, 8, 9, 10, 20, 30, 40, 50, 60, 70, 80, 90, or at least 100 fold in an ischemic stroke sample when compared to a polynucleotide reference profile. In some cases, a polynucleotide can be identified as a biomarker when an expression level difference in the polynucleotide decreases at least 0.5, 1, 1.5, 2, 3, 4, 5, 6, 7, 8, 9, 10, 20, 30, 40, 50, 60, 70, 80, 90, or at least 100 fold in an ischemic stroke sample when compared to a polynucleotide reference profile.

Identifying biomarkers of ischemic stroke can comprise analyzing a profile of polypeptides from an ischemic stroke sample. Analyzing a profile of polypeptides can comprise comparing the profile of polypeptides to a polypeptides reference profile. In some cases, comparing a profile of polypeptides to a reference profile can comprise determining expression level differences between the polypeptides in an ischemic stroke sample and the polypeptides in a reference profile. When the expression level of a polypeptide in an ischemic stroke sample is up-regulated or down-regulated compared to the expression level of the polypeptide in a reference profile, the polypeptide can be a biomarker. Such biomarker can be associated with ischemic stroke. In some cases, further analysis can be carried out to identify the biomarker as a biomarker of ischemic stroke. A polypeptide can be identified as a biomarker when an expression level difference in the polynucleotide of at least 0.5, 1, 1.5, 2, 3, 4, 5, 6, 7, 8, 9, 10, 20, 30, 40, 50, 60, 70, 80, 90, or at least 100 fold is detected in an ischemic stroke sample when compared to a polypeptide reference profile. In some cases, a polypeptide can be identified as a biomarker when an expression level difference in the polypeptide increases at least 0.5, 1, 1.5, 2, 3, 4, 5, 6, 7, 8, 9, 10, 20, 30, 40, 50, 60, 70, 80, 90, or at least 100 fold in an ischemic stroke sample when compared to a polypeptide reference profile. In some cases, a polypeptide can be identified as a biomarker when an expression level difference in the polypeptide decreases at least 0.5, 1, 1.5, 2, 3, 4, 5, 6, 7, 8, 9, 10, 20, 30, 40, 50, 60, 70, 80, 90, or at least 100 fold in an ischemic stroke sample when compared to a polypeptide reference profile.

In some aspects, analyzing a profile of biomarkers may comprise using multivariate statistical analysis.

Methods for identifying biomarkers of ischemic stroke can comprise one or more of

a) measuring expression of a group of genes in a ischemic stroke sample and expression of the group of genes in a non-ischemic stroke sample, wherein the measuring is performed by an immunoassay, polymerase chain reaction, or a combination thereof; b) analyzing the expression of the group of genes in the ischemic stroke sample and the expression of the group of genes in the non-ischemic stroke sample, thereby identifying a plurality of subgroups of genes predicative of ischemic stroke; and c) designating a gene in the group of genes as the biomarker if the gene is included in the subgroups identified in b) for a number of times that exceeds a reference value.

Biomarkers of ischemic stroke can be identified using methods such as machine learning and or pattern recognition. In some cases, biomarkers of ischemic stroke can be identified by based on a predictive model. Established statistical algorithms and methods useful as models or useful in designing predictive models, can include but are not limited to: analysis of variants (ANOVA); Bayesian networks; boosting and Ada-boosting; bootstrap aggregating (or bagging) algorithms; decision trees classification techniques, such as Classification and Regression Trees (CART), boosted CART, Random Forest (RF), Recursive Partitioning Trees (RPART), and others; Curds and Whey (CW); Curds and Whey-Lasso; dimension reduction methods, such as principal component analysis (PCA) and factor rotation or factor analysis; discriminant analysis, including Linear Discriminant Analysis (LDA), Eigengene Linear Discriminant Analysis (ELDA), and quadratic discriminant analysis; Discriminant Function Analysis (DFA); factor rotation or factor analysis; genetic algorithms; Hidden Markov Models; kernel based machine algorithms such as kernel density estimation, kernel partial least squares algorithms, kernel matching pursuit algorithms, kernel Fisher's discriminate analysis algorithms, and kernel principal components analysis algorithms; linear regression and generalized linear models, including or utilizing Forward Linear Stepwise Regression, Lasso (or LASSO) shrinkage and selection method, and Elastic Net regularization and selection method; glmnet (Lasso and Elastic Net-regularized generalized linear model); Logistic Regression (Log Reg); meta-learner algorithms; nearest neighbor methods for classification or regression, e.g. Kth-nearest neighbor (KNN); non-linear regression or classification algorithms; neural networks; partial least square; rules based classifiers; shrunken centroids (SC); sliced inverse regression; Standard for the Exchange of Product model data, Application Interpreted Constructs (StepAIC); super principal component (SPC) regression; and, Support Vector Machines (SVM) and Recursive Support Vector Machines (RSVM), among others. Additionally, clustering algorithms can also be used in determining subject sub-groups. In some cases, classification methods can be used to identify biomarkers of ischemic stroke. Such classification methods include support vector machine (SVM), k-nearest neighbors (kNN), and classification trees (Hastie, et al. (2001) The Elements of Statistical Learning, Springer, N.Y.). 10-fold cross validation can be used to evaluate the classification accuracy.

In some cases, biomarkers of ischemic stroke can be identified using Genetic Algorithm-K Nearest Neighbors (GA-kNN), a pattern recognition approach designed to identify sets of predictive variables which can optimally discriminate between classes of samples. Analysis of high dimensional genomic datasets using the GA-kNN method has been successfully used in fields such as cancer biology and toxicology to identify diagnostically relevant biomarker panels with powerful predictive ability.

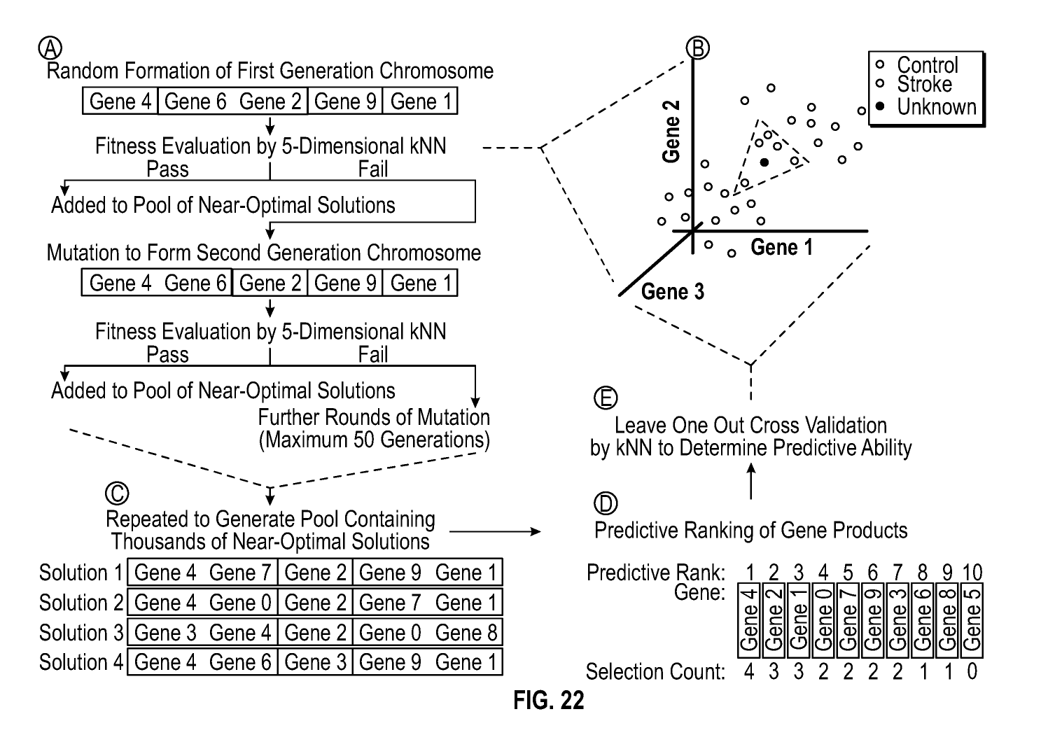

The GA/kNN approach can combine a powerful search heuristic, GA, with a non-parametric classification method, kNN. In GA/kNN analysis, a small combination of genes (referred to as a chromosome) can be generated by random selection from the total pool of gene expression data (FIG. 22, step A). The ability of this randomly generated chromosome to predict sample class can be then evaluated using kNN. In this kNN evaluation, each sample can be plotted as a vector in an n^{th} dimensional space, with the coordinates of each vector being comprised of the expression levels of the genes of the chromosome. The class of each sample can be then predicted based on the majority class of the other samples which lie closest in Euclidian distance, which can be referred to the nearest neighbors (FIG. 22, step B). The predictive ability of the chromosome can be quantified as a fitness score, or the proportion of samples which the chromosome can be correctly able to predict. A termination cutoff (minimum proportion of correct predications) can determine the level of fitness required to pass evaluation. A chromosome which passes kNN evaluation can be identified as a near-optimal solution and can be recorded, while a chromosome which fails evaluation can undergo mutation and can be re-evaluated. This process of mutation and re-evaluation can be repeated until the fitness score of the chromosome exceeds the termination cutoff (FIG. 22, step A). This process can be repeated multiple times (typically thousands) to generate a pool of heterogeneous near-optimal solutions (FIG. 22, step C). The predicative ability of each gene in the total pool of gene expression can be then ranked according to the number of times it is part of a near-optimal solution (FIG. 22 step D). The collective predictive ability of the top ranked genes can then be tested in a leave one out cross validation (FIG. 22, step E).

As used herein, the terms “reference” and “reference profile” can be used interchangeably to refer to a profile (e.g., expression) of biomolecules in a reference subject. In some cases, a reference can be the expression of a group of biomarkers in a reference subject. A reference or reference profile can be a profile of polynucleotides or a profile of polypeptides in a reference subject. In some cases, a reference subject can be a stroke subject. In some cases, a reference subject can be a non-stroke subject. In some cases, a reference subject can be a non-ischemic stroke subject. In some cases, a non-ischemic stroke can be a subject who has no ischemic stroke but has a transient ischemic attack, a non-ischemic stroke, or a stroke mimic. A subject having a non-ischemic stroke can have hemorrhagic stroke. When comparing profiles of polynucleotides and/or polypeptides in an ischemic stroke subject to profiles of the biomolecules in a reference subject, the following groups of subjects can be used: (1) ischemic stroke; (2) hemorrhagic stroke; (3) normals; (4) TIAs; (5) other stroke mimics. One would measure profiles of biomolecules for all the subjects. Then, the members of any one of these 5 groups can be compared to the profiles of the members of any other of these groups to define a function and weighting factor that best differentiates these groups based on the measured profiles. This can be repeated as all 5 groups are compared pairwise. A reference profile can be stored in computer readable form. In some aspects, a reference profile can be stored in a database or a server. In some cases, a reference can be stored in a database that is accessible through a computer network (e.g., Internet). In some cases, a reference can be stored and accessible by Cloud storage technologies.

A biomarker disclosed above can be identified as a biomarker of ischemic stroke with further analysis. In some cases, a polynucleotide biomarker that is up-regulated in an ischemic stroke sample compared to a reference profile can be identified as a biomarker of ischemic stroke if the protein or polypeptide encoded by the polynucleotide biomarker is also up-regulated in the ischemic stroke sample compared to a protein or polypeptide reference profile. For example, a polynucleotide biomarker that is up-regulated in an ischemic stroke sample compared to a reference profile can be identified as a biomarker of ischemic stroke if the protein or polypeptide encoded by the polynucleotide biomarker is also up-regulated at least about 0.5, 1, 1.5, 2, 3, 4, 5, 6, 7, 8, 9, 10, 20, 30, 40, 50, 60, 70, 80, 90, or at least 100 fold in an ischemic stroke sample compared to a protein or polypeptide reference profile. In some cases, a polynucleotide biomarker that is down-regulated in an ischemic stroke sample compared to a reference profile can be identified as a biomarker of ischemic stroke if the protein or polypeptide encoded by the polynucleotide biomarker is also down-regulated in ischemic stroke sample compared to a protein reference profile. For example, a polynucleotide biomarker that is down-regulated in an ischemic stroke sample compared to a reference profile can be identified as a biomarker of ischemic stroke if the protein or polypeptide encoded by the polynucleotide biomarker is also down-regulated at least about 0.5, 1, 1.5, 2, 3, 4, 5, 6, 7, 8, 9, 10, 20, 30, 40, 50, 60, 70, 80, 90, or at least 100 fold in an ischemic stroke sample compared to a protein or polypeptide reference profile. In some cases, a polypeptide that is up-regulated in an ischemic stroke sample compared to a reference profile can be identified as a biomarker of ischemic stroke if the polynucleotide encoding the polypeptide biomarker is also up-regulated in the ischemic stroke sample compared to a protein reference profile. For example, a polypeptide biomarker that is up-regulated in an ischemic stroke sample compared to a reference profile can be identified as a biomarker of ischemic stroke if the polynucleotide encoding the polynucleotide biomarker is also up-regulated at least about 0.5, 1, 1.5, 2, 3, 4, 5, 6, 7, 8, 9, 10, 20, 30, 40, 50, 60, 70, 80, 90, or at least 100 fold in the ischemic stroke sample compared to a polynucleotide reference profile. In some cases, a polypeptide biomarker that is down-regulated in an ischemic stroke sample compared to a reference profile can be identified as a biomarker of ischemic stroke if the polynucleotide biomarker encoding the polypeptide is also down-regulated in the ischemic stroke sample compared to a protein reference profile. For example, a polypeptide biomarker that is down-regulated in an ischemic stroke sample compared to a reference profile can be identified as a biomarker of ischemic stroke if the polynucleotide encoding the polynucleotide biomarker is also down-regulated at least about 0.5, 1, 1.5, 2, 3, 4, 5, 6, 7, 8, 9, 10, 20, 30, 40, 50, 60, 70, 80, 90, or at least 100 fold in the ischemic stroke sample compared to a polynucleotide reference profile.

Methods herein can further comprise determining the effectiveness of a given biomarker (e.g., biomarkers of ischemic stroke) or a given group of biomarkers (e.g., biomarkers of ischemic stroke). Parameters to be measured include those described in Fischer et al., Intensive Care Med. 29: 1043-51, 2003, which is incorporated herein in its entirety. These parameters include sensitivity and specificity, predictive values, likelihood ratios, diagnostic odds ratios, and receiver operating characteristic (ROC) curve areas. One or a group of effective biomarkers can exhibit one or more of the following results on these various parameters: at least 75% sensitivity, combined with at least 75% specificity; ROC curve area of at least 0.7, at least 0.8, at least 0.9, or at least 0.95; and/or a positive likelihood ratio (calculated as sensitivity/(1-specificity)) of at least 5, at least 10, or at least 20, and a negative likelihood ratio (calculated as (1-sensitivity)/specificity) of less than or equal to 0.3, less than or equal to 0.2, or less than or equal to 0.1. The ROC areas can be calculated and used in determining the effectiveness of a biomarker as described in US Patent Application Publication No. 2013/0189243, which is incorporated herein in its entirety.

Methods, devices and kits provided herein can assess a condition (e.g., ischemic stroke or a risk of ischemic stroke) in a subject with high specificity and sensitivity. As used herein, the term “specificity” can refer to a measure of the proportion of negatives that are correctly identified as such (e.g., the percentage of healthy people who are correctly identified as not having the condition). As used herein, the term “sensitivity” can refer to a measure of the proportion of positives that are correctly identified as such (e.g., the percentage of sick people who are correctly identified as having the condition). Methods, devices and kits provided herein can assess a condition (e.g., ischemic stroke) in a subject with a specificity of at least about 75%, 80%, 85%, 90%, 95%, 96%, 97%, 98%, 99%, or 100%. Methods, devices and kits provided herein can assess a condition (e.g., ischemic stroke) in a subject with a sensitivity of at least about 75%, 80%, 85%, 90%, 95%, 96%, 97%, 98%, 99%, or 100%. Methods, devices and kits provided herein can assess a condition (e.g., ischemic stroke) in a subject with a specificity of at least about 70% and a sensitivity of at least about 70%, a specificity of at least about 75% and a sensitivity of at least about 75%, a specificity of at least about 80% and a sensitivity of at least about 80%, a specificity of at least about 85% and a sensitivity of at least about 85%, a specificity of at least about 90% and a sensitivity of at least about 90%, a specificity of at least about 95% and a sensitivity of at least about 95%, a specificity of at least about 96% and a sensitivity of at least about 96%, a specificity of at least about 97% and a sensitivity of at least about 97%, a specificity of at least about 98% and a sensitivity of at least about 98%, a specificity of at least about 99% and a sensitivity of at least about 99%, or a specificity of about 100% a sensitivity of about 100%.

Methods of assessing a condition in a subject herein can achieve high specificity and sensitivity based on the expression of various numbers of biomarkers. In some cases, the methods of assessing a condition in a subject can achieve a specificity of at least about 70% and a sensitivity of at least about 70%, a specificity of at least about 75% and a sensitivity of at least about 75%, a specificity of at least about 80% and a sensitivity of at least about 80%, a specificity of at least about 85% and a sensitivity of at least about 85%, a specificity of at least about 90% and a sensitivity of at least about 90%, a specificity of at least about 95% and a sensitivity of at least about 95%, a specificity of at least about 96% and a sensitivity of at least about 96%, a specificity of at least about 97% and a sensitivity of at least about 97%, a specificity of at least about 98% and a sensitivity of at least about 98%, a specificity of at least about 99% and a sensitivity of at least about 99%, or a specificity of 100% a sensitivity of 100% based on the expression of no more than 2, 3, 4, 5, 6, 7, 8, 9, or 10 biomarkers. In some cases, the methods, devices and kits of assessing a condition in a subject can achieve a specificity of at least about 92% and a sensitivity of at least about 92%, a specificity of at least about 95% and a sensitivity of at least about 95%, a specificity of at least about 96% and a sensitivity of at least about 96%, a specificity of at least about 97% and a sensitivity of at least about 97%, a specificity of at least about 98% and a sensitivity of at least about 98%, a specificity of at least about 99% and a sensitivity of at least about 99%, or a specificity of about 100% and a sensitivity of about 100% based on the expression of two biomarkers. In some cases, the methods of assessing a condition in a subject can comprise measuring the expression of two or more of ANTXR2, STK3, PDK4, CD163, MAL, GRAP, CTSZ, KIF1B, and PLXDC2, and the method can achieve a specificity of at least 90% and a sensitivity of at least 90%, a specificity of at least 92% and a sensitivity of at least 92%, a specificity of at least 95% and a sensitivity of at least 95%, a specificity of at least 96% and a sensitivity of at least 96%, a specificity of at least 97% and a sensitivity of at least 97%, a specificity of at least 98% and a sensitivity of at least 98%, a specificity of at least 99% and a sensitivity of at least 99%, or a specificity of 100% and a sensitivity of 100%. In some cases, the methods of assessing a condition in a subject can comprise measuring the expression of two or more (e.g., four) of ANTXR2, STK3, PDK4, CD163, and the method can achieve a specificity of at least 98% and a sensitivity of at least 98%.

Assessing ischemic stroke can comprise distinguishing a subject with ischemic stroke from a healthy subject, or a subject with stroke mimics. Methods, devices, and kits herein can achieve high specificity and sensitivity in distinguishing a subject with ischemic stroke form a healthy subject, and distinguishing the subject with ischemic stroke from a subject with stroke mimics. For example, methods, devices, and kits herein can achieve a specificity of at least 92% and a sensitivity of at least 92%, a specificity of at least 95% and a sensitivity of at least 95%, a specificity of at least 96% and a sensitivity of at least 96%, a specificity of at least 97% and a sensitivity of at least 97%, a specificity of at least 98% and a sensitivity of at least 98%, a specificity of at least 99% and a sensitivity of at least 99%, or a specificity of 100% and a sensitivity of 100% in distinguishing a subject with ischemic stroke form a healthy subject, and meanwhile can achieve a specificity of at least 92% and a sensitivity of at least 92%, a specificity of at least 95% and a sensitivity of at least 95%, a specificity of at least 96% and a sensitivity of at least 96%, a specificity of at least 97% and a sensitivity of at least 97%, a specificity of at least 98% and a sensitivity of at least 98%, a specificity of at least 99% and a sensitivity of at least 99%, or a specificity of 100% and a sensitivity of 100% in distinguishing the subject with ischemic stroke from a subject with stroke mimics.

In some cases, methods of assessing ischemic stroke (e.g., distinguish ischemic stroke from a healthy condition or a stroke mimics condition) that comprises measuring a level of cell-free nucleic acid can also achieve the specificity and sensitivity disclosed herein. For example, such methods can achieve a sensitivity of at least 80%, and a specificity of at least 75%, a sensitivity of at least 85%, and a specificity of at least 80%, a sensitivity of at least 90%, and a specificity of at least 85%, a sensitivity of at least 95%, and a specificity of at least 80%, a sensitivity of 100%, and a specificity of at least 85%, a sensitivity of 100%, and a specificity of at least 90%, a sensitivity of 100%, and a specificity of at least 95%, a sensitivity of 100%, and a specificity of 100%. In some cases, the specificity can be at least 50%, 60%, 70%, 80%, 90%. In some cases, the sensitivity can be at least 50%, 60%, 70%, 80%, 90%.

Also provided herein are methods of detecting ischemic stroke in a subject. The methods can be used to detect the absence or presence of ischemic stroke. In some cases, the methods can also be used to detect a subject's risk of having a stroke.

The methods of detecting ischemic stroke can comprise measuring a profile of a first group of biomarkers of ischemic stroke and a second group of biomarkers of ischemic stroke, wherein the first and second groups of biomarkers of ischemic stroke are different classes of biomolecules. In some cases, the first group of biomarkers can be polynucleotides and the second group of biomarkers can be polypeptides. The methods can further comprise analyzing the profile of the first and second groups of biomarkers, and detecting ischemic stroke in the subject. In some cases, the analysis can be performed by a computer system.

The biomarkers of ischemic stroke used to detect ischemic stroke can be any biomarkers of ischemic stroke identified by methods provided herein or known in the art. In some cases, the biomarkers of ischemic stroke (e.g., the first group of biomarkers of ischemic stroke) can include polynucleotides encoding at least one of CCL19, CCL21, Galectin 3, RAGE, ENA78, GMCSF, CD30, CCR7, CSPG2, IQGAP1, ORM1, ARG1, LY96, MMP9, CA4, s100A12, Nav3, SAA, IGα, IGγ, IGκ, IGλ, or an active fragment thereof. In some cases, the biomarkers of ischemic stroke (e.g., the second group of biomarkers of ischemic stroke) can include at least one of CCL19, CCL21, Galectin 3, RAGE, ENA78, GMCSF, CD30, CCR7, CSPG2, IQGAP1, ORM1, ARG1, LY96, MMP9, CA4, s100A12, Nav3, SAA, IGα, IGγ, IGκ, IGλ, or an active fragment thereof. In some cases, the biomarkers of ischemic stroke can include one or more cytokines. In some cases, the biomarkers of ischemic stroke (e.g., the first group of biomarkers of ischemic stroke) can include polynucleotides encoding at least one of BAFF, MMP9, APP, Aggrecan, Galectin 3, Fas, RAGE, Ephrin A2, CD30, TNR1, CD27, CD40, TNFα, IL6, IL8, IL10, IL1β, IFNγ, RANTES, IL1α, IL4, IL17, IL2, GMCSF, ENA78, IL5, IL12P70, TARC, GroAlpha, IL33, BLCBCA, IL31, MCP2, IGG3, IGG4, Isoform 2 of Teneurin 1, and isoform 2 of aDisintegrin or an active fragment thereof. In some cases, the biomarkers of ischemic stroke (e.g., the second group of biomarkers of ischemic stroke) can include at least one of BAFF, MMP9, APP, Aggrecan, Galectin 3, Fas, RAGE, Ephrin A2, CD30, TNR1, CD27, CD40, TNFα, IL6, IL8, IL10, IL1β, IFNy, RANTES, IL1α, IL4, IL17, IL2, GMCSF, ENA78, IL5, IL12P70, TARC, GroAlpha, IL33, BLCBCA, IL31, MCP2, TLR2, TLR4, JAK2, CCR7, AKAP7, IL10, SYK, IL8, MyD88, CD3, CD4, IL22R, IL22, CEBPB or an active fragment thereof. In some cases, biomarkers of ischemic stroke provided herein can include at least one biomarkers in Table 1, FIGS. 10A-10H or any active form thereof. In some cases, biomarkers of ischemic stroke provided herein can include polynucleotides encoding at least one biomarkers in Table 1, FIGS. 10A-10H or any active form thereof.

The profiles of biomarkers of ischemic stroke can comprise a profile of at least one biomarkers of ischemic stroke disclosed herein. In some cases, the method can comprise measuring a profile of at least 1, 2, 3, 4, 5, 6, 7, 8, 9, 10, 20, 30, 40, 50, 60, 70, 80, 90, 100, 200, 300, 400, 500, 600, 700, 800, 900, or 1000 biomarkers of ischemic stroke, wherein the biomarkers of ischemic stroke are polynucleotides, and/or measuring a profile of at least 1, 2, 3, 4, 5, 6, 7, 8, 9, 10, 20, 30, 40, 50, 60, 70, 80, 90, 100, 200, 300, 400, 500, 600, 700, 800, 900, or 1000 biomarkers of ischemic stroke, wherein the biomarkers of ischemic stroke are polypeptides. In some cases, the method can comprise measuring the profiles of the same number of polynucleotide biomarkers of ischemic stroke and polypeptide biomarkers of ischemic stroke. In some cases, the method can comprise measuring the profiles of different numbers of polynucleotide biomarkers of ischemic stroke and polypeptide biomarkers of ischemic stroke. In some cases, the method of detecting ischemic stroke can comprise measuring a profile of polynucleotides encoding one or more of LY96, ARG1, and CA4, and/or measuring a profile of one or more of LY96, ARG1, and CA4. In some cases, the method of detecting ischemic stroke can comprise measuring a profile of polynucleotides encoding one or more of CCR7, CSPG2, IQGAP1, and ORM1, and/or measuring a profile of one or more of CCR7, CSPG2, IQGAP1, and ORM1. In some cases, the method of detecting ischemic stroke can comprise measuring a profile of polynucleotides encoding one or more of CCR7, CSPG2, IQGAP1, and ORM1, and/or measuring a profile of one or more of CCR7, CSPG2, IQGAP1, and ORM1. In some cases, the method of detecting ischemic stroke can comprise measuring a profile of polynucleotides encoding one or more of CCR7, CSPG2, IQGAP1, ARG1, LY96, MMP9, CA4, and s100A12 and ORM1, and/or measuring a profile of one or more of CCR7, CSPG2, IQGAP1, ARG1, LY96, MMP9, CA4, and s100A12 and ORM1.

The method of detecting ischemic stroke can further comprise analyzing the profile of a first and second group of biomarkers of ischemic stroke disclosed herein. The analyzing can comprise comparing the profile of the first and second groups of biomarkers of ischemic stroke to their reference profiles. In some cases, the analyzing can include determining the expression level differences of the biomarkers of ischemic stroke in a sample of a subject compared to a reference profile. Ischemic stroke can be detected in the subject if the expression level differences of the biomarkers of ischemic stroke in the sample compared to the reference profile falls outside a reference value range. For example, the reference profiles can be obtained from one or more non-ischemic stroke subjects. In some cases, the analyzing can comprise comparing the profile of the biomarkers of ischemic stroke in a subject to the reference value range, and the ischemic stroke can be detected if the profile of the biomarkers falls inside the reference value range. For example, the reference value range can be pre-determined as the profile of biomarkers of ischemic stroke in an ischemic stroke subject. In some cases, the methods of detecting ischemic stroke can comprise comparing the expression patterns of a first and second group of biomarkers to their reference profiles, and detecting ischemic stroke.

The methods herein can detect ischemic stroke by analyzing profiles of more than one groups of biomarkers of ischemic stroke. In some cases, the methods can comprise analyzing profiles of two groups of biomarkers of ischemic stroke. One group of the biomarkers can comprise a class of biomolecules and the second group can comprise a different class of biomolecules. For example, ischemic stroke can be detected in a subject by analyzing a profile of polynucleotide biomarkers of ischemic stroke and a profile of polypeptide biomarkers of ischemic stroke. Ischemic stroke can be detected in a subject when the outcome of analysis of the profile of both groups of biomarkers of ischemic stroke suggests that the subject has an ischemic stroke.

Methods of assessing ischemic stroke in a subject can comprise comparing the expression of a group of biomarkers to a reference. Ischemic stroke can be indicated by a difference between the expression of one or more biomarkers in the group of biomarkers and a reference. In some cases, ischemic stroke can be indicated by increase of the expression of one or more biomarkers in the group of biomarkers, e.g., increase of at least about 0.1, 0.2, 0.4, 0.6, 0.8, 1, 1.1, 1.2, 1.3, 1.4, 1.5, 1.6, 1.7, 1.8, 1.9, 2, 2.5, 3, 4, 5, 6, 7, 8, 9, 10, 100, or 1000 fold compared to a reference. In some cases, ischemic stroke can be indicated by decrease of the expression of one or more biomarkers in the group of biomarkers, e.g., decrease of at least 0.1, 0.2, 0.4, 0.6, 0.8, 1, 1.1, 1.2, 1.3, 1.4, 1.5, 1.6, 1.7, 1.8, 1.9, 2, 2.5, 3, 4, 5, 6, 7, 8, 9, 10, 100, or 1000 fold compared to a reference. In some cases, ischemic stroke can be indicated by increase of the expression of one or more of ANTXR2, STK3, PDK4, CD163, CTSZ, KIF1B, and PLXDC2. In some cases, ischemic stroke can be indicated by decrease of the expression of one or more of MAL, GRAP, and ID3.

In some cases, ischemic stroke can be indicated by increase of the expression of a first subgroup of a group of biomarkers and decrease of the expression of a second subgroup of the group of biomarkers. The first subgroup of biomarkers can comprise at least 1, 2, 3, 4, 5, 6, 7, 8, 9, 10, 50, or 100 biomarkers, and the second subgroup of biomarkers can comprise at least 1, 2, 3, 4, 5, 6, 7, 8, 9, 10, 50, or 100 biomarkers. In some cases, the first subgroup of biomarkers can comprise 4 biomarkers, and the second subgroup of biomarkers can comprise 3 biomarkers. In certain cases, the first subgroup of biomarkers can comprise 7 biomarkers, and the second subgroup of biomarkers can comprise 3 biomarkers. In some cases, ischemic stroke can be indicated by increase of the expression of one or more of ANTXR2, STK3, PDK4, CD163, CTSZ, KIF1B, and PLXDC2 and decrease of the expression of one or more of MAL, GRAP, and ID3. In some cases, ischemic stroke can be indicated by increase of the expression of ANTXR2, STK3, PDK4, and CD163, and decrease of the expression of MAL, GRAP, and ID3. In some cases, ischemic stroke can be indicated by increase of the expression off ANTXR2, STK3, PDK4, CD163, CTSZ, KIF1B, and PLXDC2, and decrease of the expression of MAL, GRAP, and ID3.

The expression of different groups of biomarkers can be measured for assessing ischemic stroke in different groups of subjects (e.g., to achieve better specificity and sensitivity). In some cases, the expression of different groups of biomarkers can be measured for assessing subjects of different ages, genders, or ethnicities, geographical areas, or weights. In some cases, the expression of different groups of biomarkers can be measured for assessing subjects having different risk factors for stroke. For example, to achieve a specificity of greater than 90% and a sensitivity of greater than 90%, expression of biomarkers #1, #2, #3, and #4 can be measured for assessing ischemic stroke of subjects from geographic area A, and expression of biomarkers #1, #2, #5, and #6 can be measured for assessing ischemic stroke of subjects from geographic area B. In some cases, some, but not all biomarkers in the different groups of biomarkers can be the same. In some cases, no biomarker in the different groups of biomarkers is the same.

Methods of detecting ischemic stroke in a subject herein can also comprise measuring a profile of blood in the subject. A profile of blood can be a profile of blood cells. The profile of blood cells can comprise a total white blood cell count, white blood cell differential (e.g., lymphocyte and neutrophil counts), and a neutrophil/lymphocyte ratio. In some cases, the methods can comprise measuring white blood cell differential in the blood of a subject. White blood cell differential can refer to the proportions of the different types of white blood cells in the blood. In some cases, white blood cell differential can refer to the percentage or absolute number of one or more types of white blood cells. For example, a white blood cell differential can include one or more of the following: absolute neutrophil count or % neutrophils, absolute lymphocyte count or lymphocytes, absolute monocyte count or % monocytes, absolute eosinophil count or % eosinophils, and absolute basophil count or % basophils. In another example, white blood cell differential can be the percentage or absolute number of lymphocytes and neutrophils. The profile of blood cells can comprise a platelet count.

A profile of blood cells can also include the proportion or number of blood cells other than white blood cells. In some cases, a profile of blood cells can include the number or percentage of red blood cells, platelets, or a combination thereof. A profile of blood cells can be measured by other tests known in the art, including a hemoglobin level, a troponin level, a creatinine kinase level, prothrombin time, partial thromboplastin time (e.g., activated partial thromboplastin time), or any combination thereof. A profile of blood can also include hematocrit (e.g., packed cell volume), a mean corpuscular volume, mean corpuscular hemoglobin, mean corpuscular hemoglobin concentration, red blood cell distribution width, or any combination thereof. The profile of blood cells can be used together with any profile of biomarkers of ischemic stroke disclosed herein for detecting ischemic stroke in a subject. In some cases, a subject can be considered to have an ischemic stroke if analysis outcome of both the profile of a group of biomarkers of ischemic stroke and the profile of blood cells suggest that the subject has an ischemic stroke.

In some aspects, the detecting may comprise measuring the amount of creatine kinase in a sample. In some aspects, CKMB is measured.

Detecting ischemic stroke can be performed using methods that can estimate and/or determine whether or not a subject is suffering from, or is at some level of risk of developing an ischemic stroke. A skilled artisan (e.g., stroke clinician or emergency room physician) can detect a disease on the basis of one or more diagnostic indicators, e.g., a biomarker, the risk, presence, absence, or amount of which is indicative of the presence, severity, or absence of the condition, e.g., ischemic stroke.

Methods of detecting ischemic stroke in a subject can further comprise detecting a time of the ischemic stroke onset in the subject. A plurality of biomarkers and/or profile of blood can be combined into one test for efficient processing of multiple samples. In addition, one skilled in the art would recognize the value of testing multiple samples (e.g., at successive time points) from the same individual. Testing of multiple samples from the same subject can allow the identification of changes in biomarker levels over time. Increases or decreases in biomarker levels, as well as the absence of change in biomarker levels, can provide useful information about the disease status that includes identifying the approximate time from onset of the event, the presence and amount of salvageable tissue, the appropriateness of drug therapies, the effectiveness of various therapies as indicated by reperfusion or resolution of symptoms, differentiation of the various types of stroke, identification of the severity of the event, identification of the disease severity, and identification of the patient's outcome, including risk of future events.

In some embodiments, outcome can comprise temporary or permanent symptoms or afflictions. In some embodiments outcome can be an inability to move on one side of the body; weakness on one side of the body; problems with thinking, awareness, attention, learning, judgment, and memory; problems understanding or forming speech; problems with controlling or expressing emotions; numbness or strange sensations; pain in the hands and feet that worsens with movement and temperature changes; depression or a combination thereof. In some embodiments, increased or high level of cfDNA can positively correlate with a worsen outcome. In some embodiments, decreased or low level of cfDNA can positively correlate with a better outcome. In some embodiments, increased or high level a biomarker can positively correlate with a worsen outcome. In some embodiments, decreased or low level of a biomarker can positively correlate with a better outcome.

The time of ischemic stroke onset can be detected by correlating a profile of biomarkers herein and/or profile of blood with the time of ischemic stroke onset and or determining the time of onset when the time of symptom onset is unknown. The methods, devices and kits herein can detect ischemic stroke within 120 hours, 96 hours, 72 hours, 60 hours, 48 hours, 36 hours, 24 hours, 12 hours, 11 hours, 10 hours, 9 hours, 8 hours, 7 hours, 6 hours, 5 hours, 4 hours, 3 hours, 2 hours, 1 hour, or 0.5 hour from the time of ischemic stroke onset. For example, the methods can detect ischemic stroke within 4.5 hours from the onset of ischemic stroke. The time of ischemic stroke symptom onset can be determined by correlating the expression of a group of biomarkers in a sample with the time of ischemic stroke symptom onset.

Methods herein can be performed to assess a condition (e.g., ischemic stroke) in a subject within a period of time from the symptom onset of the condition in the subject. In some cases, the methods can be performed to assess ischemic stroke in a subject within a short period of time from ischemic stroke symptom onset in the subject. For example, the methods can be performed by using a point of care device that can be used to assess ischemic stroke outside of a hospital, e.g., at the home of the subject. In some cases, the methods can be performed to assess a condition in a subject within 120 hours, 96 hours, 72 hours, 60 hours, 48 hours, 36 hours, 24 hours, 12 hours, 11 hours, 10 hours, 9 hours, 8 hours, 7 hours, 6 hours, 5 hours, 4 hours, 3 hours, 2 hours, 1 hour, 30 minutes, 20 minutes, or 10 minutes from the symptom onset of the condition.

Methods herein can further comprise administering a treatment for ischemic stroke to a subject in which ischemic stroke is detected. In some cases, the methods can comprise administering a pharmaceutically effective dose of a drug or a salt thereof for treating ischemic stroke. In some embodiments, a drug for treating ischemic stroke can comprise a thrombolytic agent or antithrombotic agent. In some embodiments, a drug for treating ischemic stroke can be one or more compounds that are capable of dissolving blood clots such as psilocybin, tPA (Alteplase or Activase), reteplase (Retavase), tenecteplase (TNKasa), anistreplase (Eminase), streptoquinase (Kabikinase, Streptase) or uroquinase (Abokinase), and anticoagulant compounds, i.e., compounds that prevent coagulation and include, without limitation, vitamin K antagonists (warfarin, acenocumarol, fenprocoumon and fenidione), heparin and heparin derivatives such as low molecular weight heparins, factor Xa inhibitors such as synthetic pentasaccharides, direct thrombin inhibitors (argatroban, lepirudin, bivalirudin and ximelagatran) and antiplatelet compounds that act by inhibition of platelet aggregation and, therefore, thrombus formation and include, without cyclooxygenase inhibitors (aspirin), adenosine diphosphate receptor inhibitors (clopidrogrel and ticlopidine), phosphodiesterase inhibitors (cilostazol), glycoprotein inhibitors (Abciximab Eptifibatide, Tirofiban and Defibrotide) and adenosine uptake inhibitors (dipiridamol). The drug for treating ischemic stroke can be tissue plasminogen activator (tPA).

In some cases, a treatment can comprise endovascular therapy. In some cases, endovascular therapy can be performed after a treatment is administered. In some cases, endovascular therapy can be performed before a treatment is administered. In some cases, a treatment can comprise a thrombolytic agent. In some cases, an endovascular therapy can be a mechanical thrombectomy. In some cases, a stent retriever can be sent to the site of a blocked blood vessel in the brain to remove a clot. In some cases, after a stent retriever grasps a clot or a portion thereof, the stent retriever and the clot or portions thereof can be removed. In some cases, a catheter can be threaded through an artery up to a blocked artery in the brain. In some cases, a stent can open and grasp a clot or portions thereof, allowing for the removal of the stent with the trapped clot or portions thereof. In some cases, suction tubes can be used. In some cases, a stent can be self-expanding, balloon-expandable, and or drug eluting.

In some cases, the treatments disclosed herein of the invention may be administered by any route, including, without limitation, oral, intravenous, intramuscular, intra-arterial, intramedullary, intrathecal, intraventricular, transdermal, subcutaneous, intraperitoneal, intranasal, enteric, topical, sublingual or rectal route. A review of the different dosage forms of active ingredients and excipients to be used and their manufacturing processes is provided in “Tratado de Farmacia Galénica”, C. Faulí and Trillo, Luzán 5, S. A. de Ediciones, 1993 and in Remington's Pharmaceutical Sciences (A. R. Gennaro, Ed.), 20^{th} edition, Williams & Wilkins Pa., USA (2000). Examples of pharmaceutically acceptable vehicles are known in prior art and include phosphate buffered saline solutions, water, emulsions, such as oil/water emulsions, different types of humectants, sterile solutions, etc. The compositions that comprise said vehicles may be formulated by conventional processes which are known in prior art

In some cases, the methods can comprise administering a pharmaceutically effective dose of a drug for treating ischemic stroke within 24 hours, 12 hours, 11 hours, 10 hours, 9 hours, 8 hours, 7 hours, 6 hours, 5 hours, 4 hours, 3 hours, 2 hours, or 1 hour, 30 minutes, 20 minutes, or 10 minutes from the ischemic stroke onset. For example, the methods can comprise administering a pharmaceutically effective dose of a drug for treating ischemic stroke within 4.5 hours of ischemic stroke onset. In a particular example, the methods can comprise administering a pharmaceutically effective dose of tPA within 4.5 hours of ischemic stroke onset. In some cases, the methods can comprise determining whether or not to take the patient to neuro-interventional radiology for clot removal or intra-arterial tPA. In this particular example, the methods can comprise administering a pharmaceutically effective dose of intra-arterial tPA within 8 hours of ischemic stroke onset. In certain cases, the methods comprise administering a treatment to the subject if the level of the cell-free nucleic acids in the subject is higher than a reference level. In some embodiments, a treatment is not administered if the level of the cell-free nucleic acids in the subject is equal to or less than the reference. In some embodiments, a treatment is administered if ischemic stroke is determined.

A drug for treating ischemic stroke can alter the expression of one or more biomarkers in a subject receiving the drug. In some cases, the drug for treating ischemic stroke can at least partially increase the expression, function, or both of one or more biomarkers in a subject receiving the drug. In some cases, the drug for treating ischemic stroke can at least partially reduce or suppress the expression, function, or both of one or more biomarkers in a subject receiving the drug.

Methods herein can further comprise other applications. In some cases, the methods can further comprise predicting an outcome of the ischemic stroke in the subject. The outcome can be predicted based on the expression of a group of biomarkers or level of nucleic acids (for example cell-free nucleic acids).

The methods can assess a risk of ischemic stroke in the subject. The risk can be assessed based on the expression of a group of biomarkers. In some cases, there is a likelihood of ischemic stroke in the subject if the expression of one or more biomarkers in a group of biomarkers is increased, e.g., by at least about 0.1, 0.2, 0.4, 0.6, 0.8, 1, 1.1, 1.2, 1.3, 1.4, 1.5, 1.6, 1.7, 1.8, 1.9, 2, 2.5, 3, 4, 5, 6, 7, 8, 9, 10, 100, or 1000 fold, compared to a reference. In some cases, there is a likelihood of ischemic stroke in the subject if the expression of one or more biomarkers in a group of biomarkers is decreased, e.g., by at least about 0.1, 0.2, 0.4, 0.6, 0.8, 1, 1.1, 1.2, 1.3, 1.4, 1.5, 1.6, 1.7, 1.8, 1.9, 2, 2.5, 3, 4, 5, 6, 7, 8, 9, 10, 100, or 1000 fold, compared to a reference.

In some cases, detecting stroke, the likelihood of ischemic stroke, the risk of stroke, or the severity of stroke can be further indicated by a second assessment. The second assessment can be a clinical assessment. Such assessment can be a neuroimaging technique, including computerized tomography (CT) scan, magnetic resonance imaging MRI (e.g., Functional magnetic resonance imaging (fMRI), diffuse optical imaging, Event-related optical signal, magnetoencephalography, positron emission tomography (PET), Single-photon emission computed tomography, cranial ultrasound, or any combination thereof.

The methods of assessing ischemic stroke in a subject can be repeated at different time points to monitor ischemic stroke and or a subject. For example, the method can be repeated within 1 day, 2 days, 3 days, 4 days, 5 days, 6 days, 1 week, 2 weeks, 3 weeks, 1 month, 2 months, 3 months, 4 months, 5 months, 6 months, 7 months, 8 months, 9 months, 10 months, 11 months, 1 year, 2 years, 3 years, 4 years, 5 years, 10 years, 20 years, 30 years, 40 years, or 50 years. In some cases, the method can be repeated for at least 1, 2, 3, 4, 5, 6, 7, 8, 9, 10, 20, 40, 60, 80, or 100 times within a time period set forth above. The methods of assessing ischemic stroke can be performed following administration of a treatment to a subject. In these cases, the expression of the group of biomarkers can be determinative of the subject's response to the treatment. In some cases, the subject's response can be an adverse reaction to the treatment. In some cases, the level of cell-free nucleic acids or a subgroup of thereof in a subject is determinative of the subject's response to the treatment.

The methods can further comprise determining whether a subject is eligible for a clinical trial. For example, the expression of a group of biomarkers in a subject can be determinative at least in part for whether the subject is eligible for a clinical trial. In some cases, the subject is eligible for a clinical trial if the expression of one or more biomarkers in a group of biomarkers is increased, e.g., by at least about 0.1, 0.2, 0.4, 0.6, 0.8, 1, 1.1, 1.2, 1.3, 1.4, 1.5, 1.6, 1.7, 1.8, 1.9, 2, 2.5, 3, 4, 5, 6, 7, 8, 9, 10, 100, or 1000 fold, compared to a reference. In some cases, the subject is not eligible for a clinical trial if the expression of one or more biomarkers in a group of biomarkers is decreased, e.g., by at least about 0.1, 0.2, 0.4, 0.6, 0.8, 1, 1.1, 1.2, 1.3, 1.4, 1.5, 1.6, 1.7, 1.8, 1.9, 2, 2.5, 3, 4, 5, 6, 7, 8, 9, 10, 100, or 1000 fold, compared to a reference. In some cases, the subject can be administered with a treatment for a condition (e.g., ischemic stroke), and the expression of a group of biomarkers can be measured. The expression of the group of biomarkers can be determinative of the subject's response to the treatment. In some cases, the level of cell-free nucleic acids or a subgroup of thereof in a subject is determinative of the subject's response to the treatment. The level of response can be used to determine whether the subject is eligible for a clinical trial.

The methods can comprise predicting a response of a subject suspected of having ischemic stroke to a treatment. Such methods can comprise one or more of the following: measuring expression of a group of biomarkers in a sample from the subject; comparing the expression of the group of biomarkers to a reference; administering the treatment to the subject; and predicting the response of the subject to the treatment. The prediction can be made by analyzing the difference between the expression of the group of biomarkers and a reference.

The method can comprise evaluating a drug (e.g., evaluating the efficiency of a drug). Such methods can comprise one or more of the following: measuring expression of a group of biomarkers in a sample from the subject; administering the drug to the subject; measuring the expression of the group of biomarkers in a second sample, where the second sample is obtained from the subject after the subject is administered the drug; comparing the expression of the group of biomarkers in the first sample and the expression of the group of biomarkers in the second sample; and evaluating the drug. The evaluation can be performed by analyzing difference between the expression of the group of biomarkers in the first sample and the expression of the group of biomarkers in the second sample.

The methods can assess the severity of a condition in a subject. In some cases, the methods can assess the severity of ischemic stroke. The methods can comprise measuring the expression of a group of biomarkers. The assessment can be made based on the expression of the group of biomarkers, e.g., by comparing the expression of biomarkers to a reference. For example, the difference between the expression of the biomarkers and the reference can be indicative of the severity of ischemic stroke. In some cases, the difference between the expression of biomarkers and the reference can be correlated with a scale of ischemic stroke severity. For example, the reference can have a reference range of the expression levels of the biomarkers from subject with ischemic stroke of certain severity. If the expression levels of the biomarkers in a subject fall into a reference range correlated to a severity level, the subject can be determined to have ischemic stroke of that severity. The scale of ischemic stroke severity can be any scale known in the art, including National Institutes of Health Stroke Scale (NIESS), Canadian neurological scale, European Stroke scale, Glasgow Coma Scale, Hemispheric Stroke Scale, Hunt & Hess Scale, Mathew Stroke Scale, Orgogozo Stroke Scale, Oxfordshire Community Stroke Project Classification, and Scandinavian Stroke Scale. In some cases, stroke severity increases as the level of one or more biomarkers increases in a sample. In some cases, stroke severity decreases as the level of one or more biomarkers increases in a sample.

The analysis of profiles of biomarkers of ischemic stroke can be carried out to optimize clinical sensitivity or specificity in various clinical settings. These include ambulatory, urgent care, emergency care, critical care, intensive care, monitoring unit, inpatient, outpatient, physician office, medical clinic, and health screening settings. Furthermore, one skilled in the art can use a single biomarker or a subset of biomarkers comprising a larger panel of biomarkers in combination with an adjustment of the diagnostic threshold in each of the aforementioned settings to optimize clinical sensitivity and specificity.

Profiles of biomarkers of ischemic stroke can be measured in a variety of physical formats as well. For example, microtiter plates or automation can be used to facilitate the processing of large numbers of test samples. Alternatively, single sample formats can be developed to facilitate immediate treatment and diagnosis in a timely fashion, for example, in ambulatory transport or emergency room settings.

Profiles of biomarkers of ischemic stroke can be measured and analyzed using any methods of measuring and analyzing profiles of biomarkers herein. In some cases, a number of immunoassays or nucleic acid based tests can be used to rapidly detect the presence of the biomarkers of ischemic stroke herein in a biological sample, in particular, when done in the context of the urgent clinical setting. Examples include radioimmunoassays, enzyme immunoassays (e.g. ELISA), immunofluorescence, immunoprecipitation, latex agglutination, hemagglutination, and histochemical tests. A particularly preferred method, however, because of its speed and ease of use, is latex agglutination. Latex agglutination assays have been described in Beltz, G. A. et al., in Molecular Probes: Techniques and Medical Applications, A. Albertini et al., eds., Raven Press, New York, 1989, incorporated herein by reference. In the latex agglutination assay, antibody raised against a particular biomarker can be immobilized on latex particles. A drop of the latex particles can be added to an appropriate dilution of the serum to be tested and mixed by gentle rocking of the card. With samples lacking sufficient levels of the biomarkers, the latex particles remain in suspension and retain a smooth, milky appearance. However, if biomarkers reactive with the antibody are present, the latex particles clump into visibly detectable aggregates. An agglutination assay can also be used to detect biomarkers wherein the corresponding antibody is immobilized on a suitable particle other than latex beads, for example, on gelatin, red blood cells, nylon, liposomes, gold particles, etc. The presence of antibodies in the assay causes agglutination, similar to that of a precipitation reaction, which can then be detected by such techniques as nephelometry, turbidity, infrared spectrometry, visual inspection, colorimetry, and the like. The term latex agglutination is employed generically herein to refer to any method based upon the formation of detectable agglutination, and is not limited to the use of latex as the immunosorbent substrate. While preferred substrates for the agglutination are latex based, such as polystyrene and polypropylene, particularly polystyrene, other well-known substrates include beads formed from glass, paper, dextran, and nylon. The immobilized antibodies may be covalently, ionically, or physically bound to the solid-phase immunosorbent, by techniques such as covalent bonding via an amide or ester linkage, ionic attraction, or by adsorption. Those skilled in the art will know many other suitable carriers for binding antibodies, or will be able to ascertain such, using routine experimentation.

### Kit of Detecting Ischemic Stroke

Provided herein also include kits of detecting ischemic stroke in a subject. The kits can be used for performing any methods described herein. For example, the kits can be used to assess a condition (e.g., ischemic stroke) in a subject. When assessing the condition with the kits, any specificity and sensitivity disclosed herein can be achieved. The kits can also be used to evaluate a treatment of a condition. For example, kits disclosed herein can comprise a panel of probes and a detecting reagent.

The kits can comprise a probe for measuring a level of cell-free nucleic acids in a sample from the subject. The probe can bind (e.g., directly or indirectly) to at least one of the cell-free nucleic acid in the sample. In some cases, the kits can comprise a probe for measuring a level of cell-free nucleic acids carrying an epigenetic marker in a sample from the subject, wherein the probe binds to the cell-free nucleic acids carrying the epigenetic marker. The kit can further comprise a detecting reagent to examining the binding of the probe to at least one of the cell-free nucleic acids.

The kits can comprise a plurality of probes that can detect one or more biomarkers of ischemic stroke. In some cases, the kits can comprise a first panel of probes for detecting at least one of a first group of biomarkers of ischemic stroke and a second panel of probes for detecting at least one of a second group of biomarkers of stroke. In some cases, the first group of biomarkers can comprise a first class of biomolecules and the second group of biomarkers can comprise a second class of biomolecules. In some cases, the first and second class of biomolecules can be different classes of biomolecules. For example, the first class of biomolecules can be polynucleotides. In another example, the second class of biomolecules can be polypeptides. In another example, the first class of biomolecules can be polynucleotides and the second class of biomolecules can be polypeptides.

The kits can comprise one or more probes that can bind one or more biomarkers of ischemic stroke. In some cases, the probes can be oligonucleotides capable of binding to the biomarkers of ischemic stroke. The biomarkers of ischemic stroke bounded by the oligonucleotides can be polynucleotides, polypeptides or proteins. In some cases, the probes in the kits can be oligonucleotides capable of hybridizing to at least one of the biomarkers of ischemic stroke (e.g., biomarkers of ischemic stroke that are polynucleotides). The oligonucleotides can be any type of nucleic acids including DNA, RNA or hybridization thereof. The oligonucleotides can be any length. In some cases, the probes herein can be other types of molecules, including aptamers.

The probes can also be proteinaceous materials, e.g., polypeptides or polypeptide fragments of the biomarkers of the invention. In some cases, the probe may be a proteinaceous compound. There is a wide variety of protein-protein interactions; however, proteins also bind nucleic acids, metals and other non-proteinaceous compounds (e.g., lipids, hormones, transmitters). Some other examples of proteins that may be used as either targets or probes include antibodies, enzymes, receptors, and DNA- or RNA-binding proteins. Both antibody and antigen preparations can be in a suitable titrated form, with antigen concentrations and/or antibody titers given for easy reference in quantitative applications.

The probes can be antibodies capable of specifically binding at least one of the biomarkers of ischemic stroke. An antibody that “specifically binds to” or is “specific for” a particular polypeptide or an epitope on a particular polypeptide can be one that binds to that particular polypeptide or epitope on a particular polypeptide without substantially binding to any other polypeptide or polypeptide epitope. Alternatively, an antibody that specifically binds to an antigen, in accordance with this invention, refers to the binding of an antigen by an antibody or fragment thereof with a dissociation constant (IQ) of 104 or lower, as measured by a suitable detection instrument, e.g., surface plasmon resonance analysis using, for example, a BIACORE® surface plasmon resonance system and BIACORE® kinetic evaluation software (eg. version 2.1). The affinity or dissociation constant (K_{d}) for a specific binding interaction is preferably about 500 nM or lower, more preferably about 300 nM or lower and preferably at least 300 nM to 50 pM, 200 nM to 50 pM, and more preferably at least 100 nM to 50 pM, 75 nM to 50 pM, 10 nM to 50 pM.

The probes can be labeled. For example, the probes can comprise labels. The labels can be used to track the binding of the probes with biomarkers of ischemic stroke in a sample. The labels can be fluorescent or luminescent tags, metals, dyes, radioactive isotopes, and the like. Examples of labels include paramagnetic ions, radioactive isotopes; fluorochromes, metals, dyes, NMR-detectable substances, and X-ray imaging compounds. Paramagnetic ions include chromium (III), manganese (II), iron (III), iron (II), cobalt (II), nickel (II), copper (II), neodymium (II), samarium (III), ytterbium (III), gadolinium (III), vanadium (II), terbium (III), dysprosium (III), holmium (III) and/or erbium (III), with gadolinium being particularly preferred. Ions useful in other contexts, such as X-ray imaging, include but are not limited to lanthanum (III), gold (III), lead (II), and especially bismuth (III). Radioactive isotopes include ¹⁴-carbon, ¹⁵chromium, ³⁶-chlorine, ⁵⁷cobalt, and the like may be utilized. Among the fluorescent labels contemplated for use include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red. Enzymes (an enzyme tag) that will generate a colored product upon contact with a chromogenic substrate may also be used. Examples of suitable enzymes include urease, alkaline phosphatase, (horseradish) hydrogen peroxidase or glucose oxidase. Secondary binding ligands can be biotin and/or avidin and streptavidin compounds. The use of such labels is well known to those of skill in the art and is described, for example, in U.S. Pat. Nos. 3,817,837; 3,850,752; 3,939,350; 3,996,345; 4,277,437; 4,275,149 and 4,366,241; each incorporated herein by reference.

The probes disclosed herein can be used to measure the expression of a group of biomarkers in methods of assessing ischemic stroke. In some cases, probes used to measure the expression of a group in methods of assessing ischemic stroke can be labeled probes that comprise any labels described herein. In some cases, the probes can be synthetic, e.g., synthesized in vitro. In some cases, the probes can be different from any naturally occurring molecules.

The probes can comprise one or more polynucleotides. In some cases, the probes can comprise polynucleotides that bind (e.g., hybridize) with the group of biomarkers. In some case, the probes can comprise polynucleotides that bind (e.g., hybridize) with the RNA (e.g., mRNA or miRNA) of the group of biomarkers. In some cases, the probes can comprise polynucleotides that bind (e.g., hybridize) with DNA derived (e.g., reversely transcribed) from RNA (e.g., mRNA or miRNA) of the group of biomarkers.

The probes can comprise polypeptides. In some cases, the probes can comprise polypeptides that bind to the proteins (or fragments of the proteins) of the group of biomarkers. Such probes can be antibodies or fragments thereof.

The probes can also comprise any other molecules that bind to the group of biomarkers other than polynucleotides or polypeptides. For example, the probes can be aptamers or chemical compounds. In some cases, the probes can comprise a combination of polynucleotides, polypeptides, aptamers, chemical compounds, and any other type of molecules.

The kits can further comprise a detecting reagent. The detecting reagent can be used for examining binding of the probes with the group of biomarkers. The detecting reagent can comprise any label described herein, e.g., a fluorescent or radioactive label. In some cases, the kits can also include an immunodetection reagent or label for the detection of specific immunoreaction between the provided biomarkers and/or antibody, as the case may be, and the diagnostic sample. Suitable detection reagents are well known in the art as exemplified by radioactive, enzymatic or otherwise chromogenic ligands, which are typically employed in association with the antigen and/or antibody, or in association with a second antibody having specificity for first antibody. Thus, the reaction can be detected or quantified by means of detecting or quantifying the label. Immunodetection reagents and processes suitable for application in connection with the novel methods of the present invention are generally well known in the art.

The reagents can include ancillary agents such as buffering agents and protein stabilizing agents, e.g., polysaccharides and the like. The kit may further include where necessary agents for reducing background interference in a test, agents for increasing signal, apparatus for conducting a test, calibration curves and charts, standardization curves and charts, and the like.

The kits can further comprise a computer-readable medium for assessing a condition in a subject. For example, the computer-readable medium can analyze the difference between the expression of the group of biomarkers in a sample from a subject and a reference, thus assessing a condition in the subject. In some embodiments, a kit disclosed herein can comprise instructions for use.

### Device of Detecting Ischemic Stroke

Disclosed herein are devices for assessing ischemic stroke in a subject. Such devices can comprise a memory that stores executable instructions. The devices can further comprise a processor that executes the executable instructions to perform the methods disclosed herein.

Disclosed herein further include devices of detecting ischemic stroke in a subject. The devices can comprise a memory that stores executive instruction and a processor that executes the executable instructions. The devices can be configured to perform any method of detecting ischemic stroke disclosed herein.

The devices can comprise immunoassay devices for measuring profiles of polypeptides or proteins. See, e.g., U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792, each of which is hereby incorporated by reference in its entirety. These devices and methods can utilize labeled probes in various sandwiches, competitive or non-competitive assay formats, to generate a signal that can be related to the presence or amount of an analyte of interest. Additionally, certain methods and devices, such as biosensors and optical immunoassays, can be employed to determine the presence or amount of analytes without the need for a labeled molecule. See, e.g., U.S. Pat. Nos. 5,631,171; and 5,955,377, each of which is hereby incorporated by reference in its entirety, including all tables, figures and claims. One skilled in the art can also recognize that robotic instrumentation including but not limited to Beckman ACCESS®, Abbott AXSYM®, Roche ELECSYS®, Dade Behring STRATUS® systems are among the immunoassay analyzers that are capable of performing the immunoassays taught herein.

The devices can comprise a filament-based diagnostic device. The filament-based diagnostic device can comprise a filament support which provides the opportunity to rapidly and efficiently move probes between different zones (e.g., chambers, such as the washing chamber or a reporting chamber) of an apparatus and still retain information about their location. It can also permit the use of very small volumes of various samples—as little as nanoliter volume reactions. The filament can be constructed so that the probes are arranged in an annular fashion, forming a probe band around the circumference of the filament. This can also permit bands to be deposited so as to achieve high linear density of probes on the filament.

The filament can be made of any of a number of different materials. Suitable materials include polystyrene, glass (e.g., fiber optic cores), nylon or other substrate derivatized with chemical moieties to impart desired surface structure (3-dimensional) and chemical activity. The filament can also be constructed to contain surface features such as pores, abrasians, invaginations, protrusions, or any other physical or chemical structures that increase effective surface area. These surface features can, in one aspect, provide for enhanced mixing of solutions as the filament passes through a solution-containing chamber, or increase the number and availability of probe molecules. The filament can also contain a probe identifier which allows the user to track large numbers of different probes on a single filament. The probe identifiers may be dyes, magnetic, radioactive, fluorescent, or chemiluminescent molecules. Alternatively, they may comprise various digital or analog tags.

The probes that are attached to the filaments can be any of a variety of biomolecules, including, nucleic acid molecules (e.g., oligonucleotides) and antibodies or antibodies fragments. The probes should be capable of binding to or interacting with a target substance of interest (e.g., the polypeptide biomarkers of the invention or their encoding mRNA molecules) in a sample to be tested (e.g., peripheral blood), such that the binding to or interaction is capable of being detected.

### EXAMPLES

### Example 1—Comparison of the Gene Expression Patterns of Biomarkers Among Ischemic Stroke Patients, Transient Ischemic Attack Patients and Stroke Mimic Patients Using PCR

Peripheral blood plasma samples from four groups of patients (i.e., 8 ischemic stroke patients, 4 transient ischemic attack (TIA) patients, 7 stroke mimic patients, and 19 control patients) were collected into PAXgene blood RNA tubes (Qiagen) within 24 hours from the onset of symptoms. The whole blood RNA was extracted and purified using the PAXgene Blood RNA Kit (Qiagen).

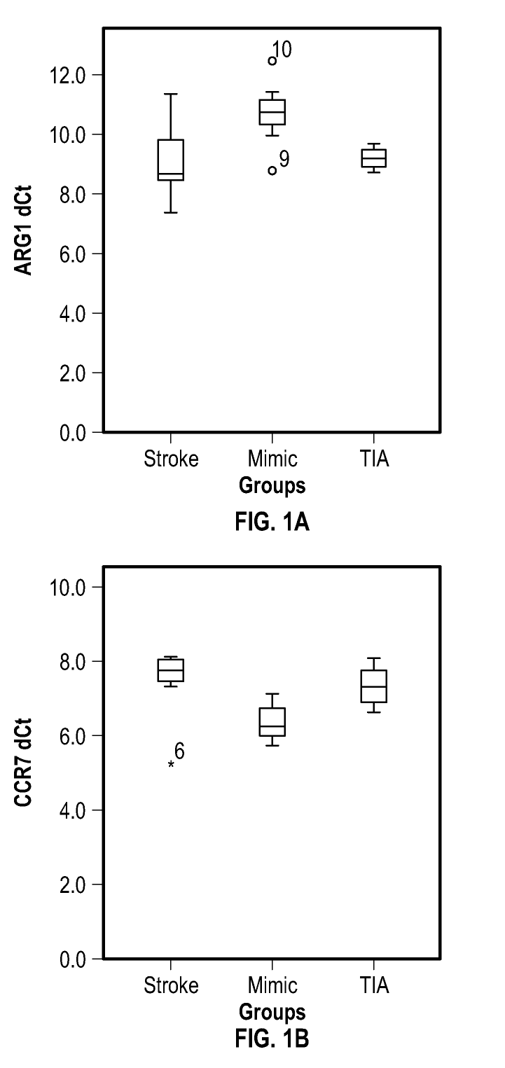

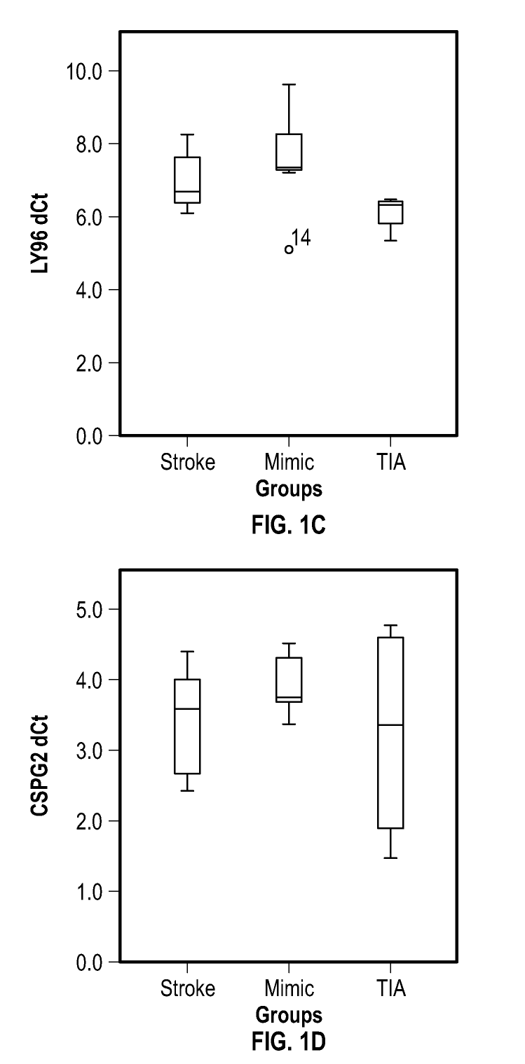

PCR was performed to measure the gene expression of ARG1, CA4, CCR7, CSPG2, IQGAP1, LY96, MMP9, ORM1 and s100a12 relative to the control group. The expression levels of ARG1 (p=0.038), CCR7 (p=0.003), LY96 (p=0.018), CSPG2 (p=0.05) were significantly different across the ischemic stroke group, the TIA group, and the stroke mimic group (FIG. 1).

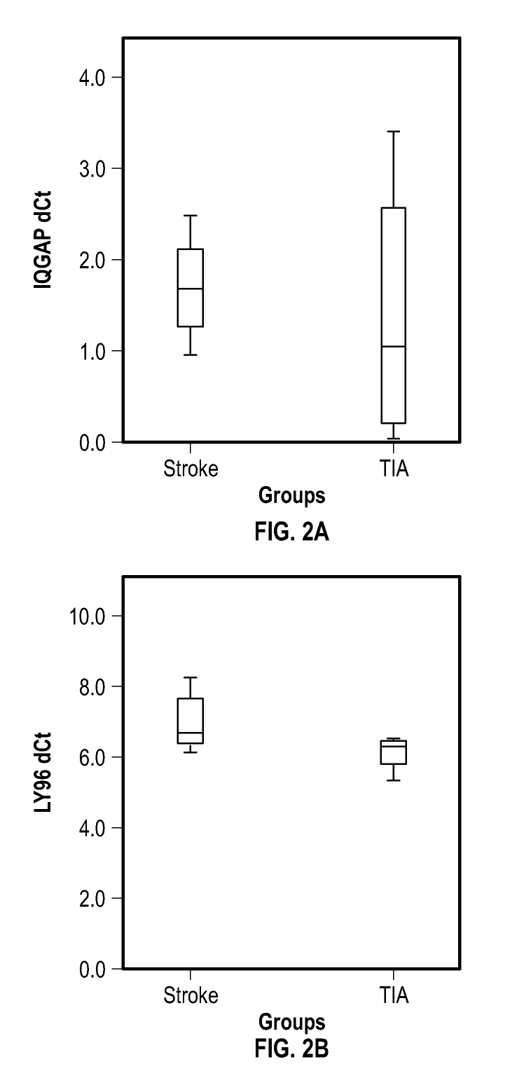

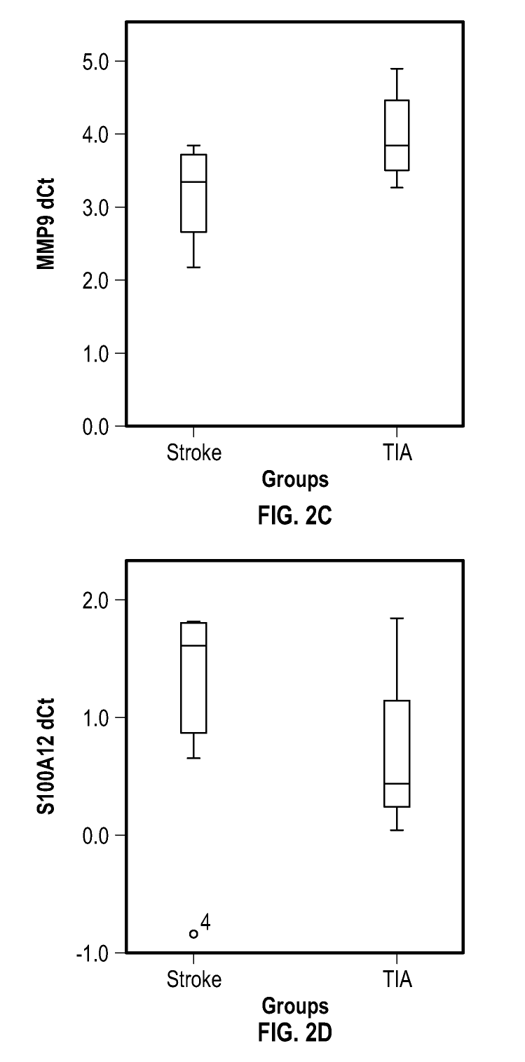

PCR was also performed to measure the gene expression of IQGAP, Ly96, MMP9, and s100a12 relative to an internal control. The expression levels of IQGAP1 (p=0.05), Ly96 (p=0.05), MMP9 (p=0.08), s100a12 (p=0.62, outlier removed for analysis) were significantly different between the ischemic stroke group and the TIA group only (FIG. 2).

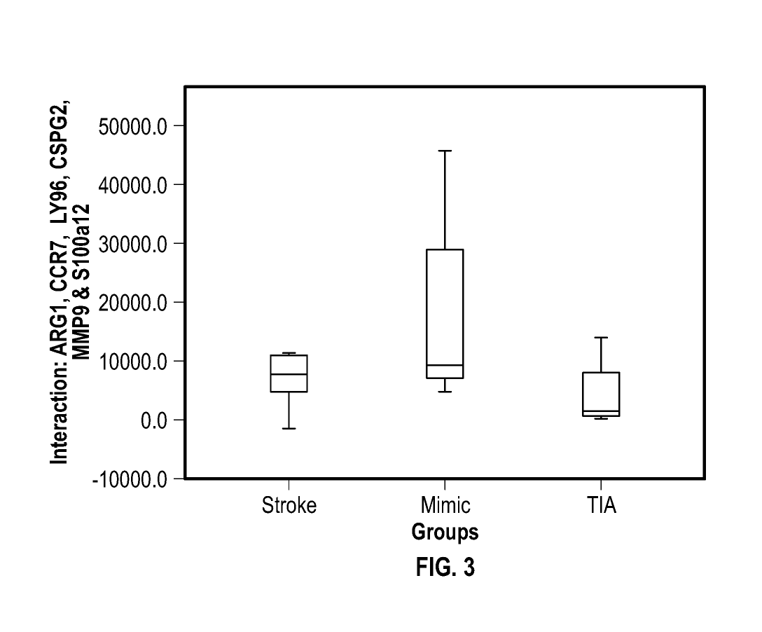

The interaction between ARG1, CCR7, LY96, CSPG2, MMP9 and s100a12 was significantly different across the ischemic stroke group, the TIA group, and the stroke mimic group (p=0.08) (FIG. 3). This interaction was a pattern of expression of all the variables of interest. Pattern recognition and machine learning analyses can be performed to fully capture the patterns of expression for each disease cohort.

The ratios between many of the genes tested herein were also significantly different among the ischemic stroke group, the TIA group, and the stroke mimic group, suggesting the patterns of expression among the three patient groups were different. The most robust statistical models for the point of care (POC) technology can be determined by further optimization.

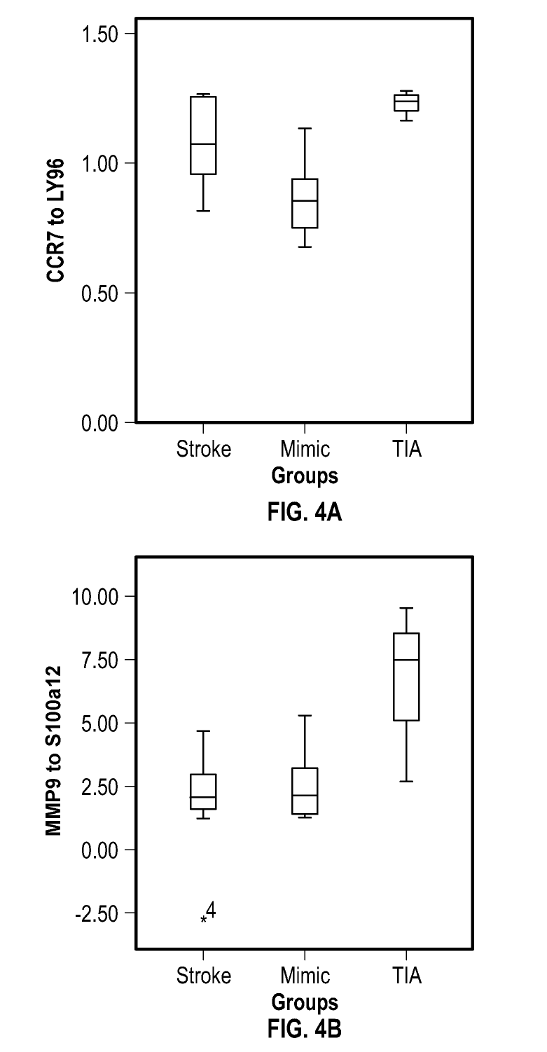

The p values from the comparison of ratios among the ischemic stroke group, the TIA group, and the stroke mimic group were as follows: ARG1 to CCR7: p=0.016; CCR7 to Ly96: p=0.008; CSPG2 to MMP9: p=0.059; IQGAP1 to MMP9: p=0.07; MMP9 to s100a12: p=0.019; ARG1 to s100a12: p=0.04; CCR7 to s100a12: p=0.05; and CA4 to s100a12: p=0.048. The ratios of CCR7 to LY96 and MMP9 to s100a12 are shown in FIGS. 4A-4B.

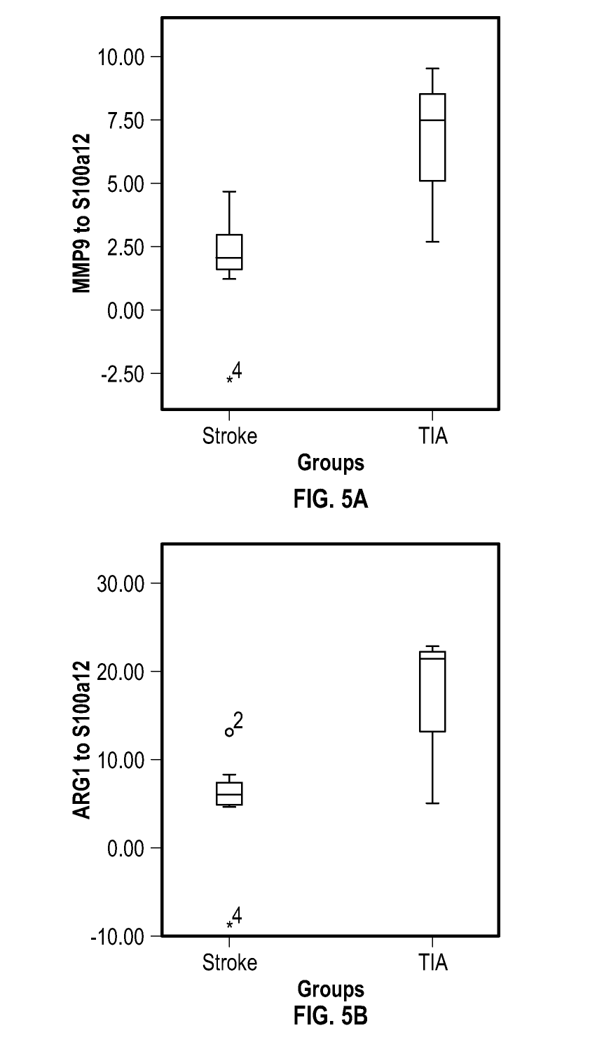

The p values from the comparison of ratios between the ischemic stroke group and the TIA group were as follows: ARG1 to LY96: p=0.09; CSPG2 to MMP9: p=0.05; IQGAP1 to MMP9: p=0.08; MMP9 to s100a12: p=0.024; ARG1 to s100a12: p=0.045; CCR7 to s100a12: p=0.067; and CA4 to s100a12: p=0.053. The ratios of MMP9 to s100a12 and ARG1 to s100a12 are shown in FIGS. 5A-5B.

### Example 2—Comparison of the Gene Expression Patterns of Biomarkers Between Ischemic Stroke Patients and Metabolic Disease Control Patients Using PCR

Peripheral blood plasma samples from 22 ischemic stroke patients and 19 metabolic disease control patients were collected in PAXgene blood RNA tubes (Qiagen) within 24 hours from the onset of symptoms. The whole blood RNA was extracted and purified using the PAXgene Blood RNA Kit.

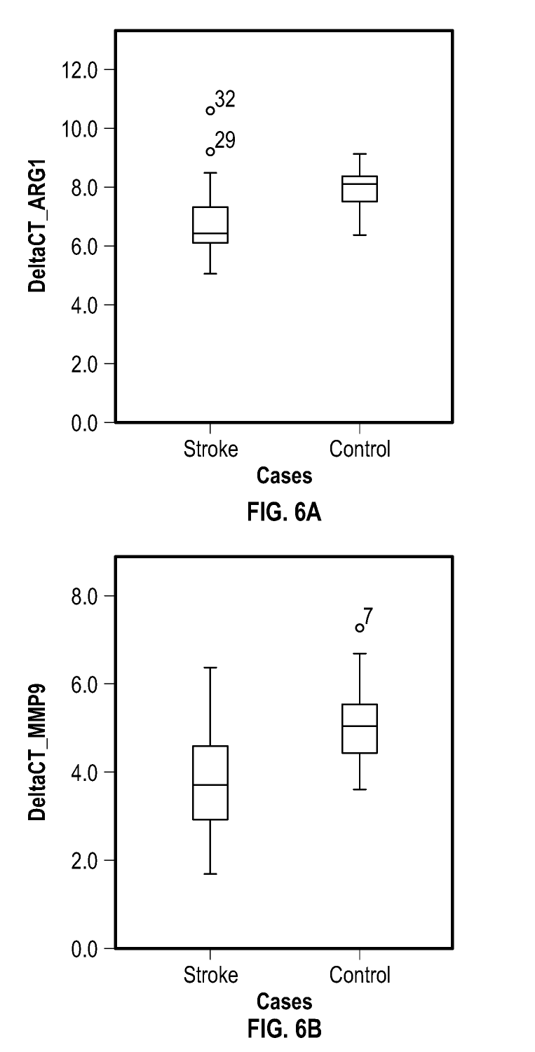

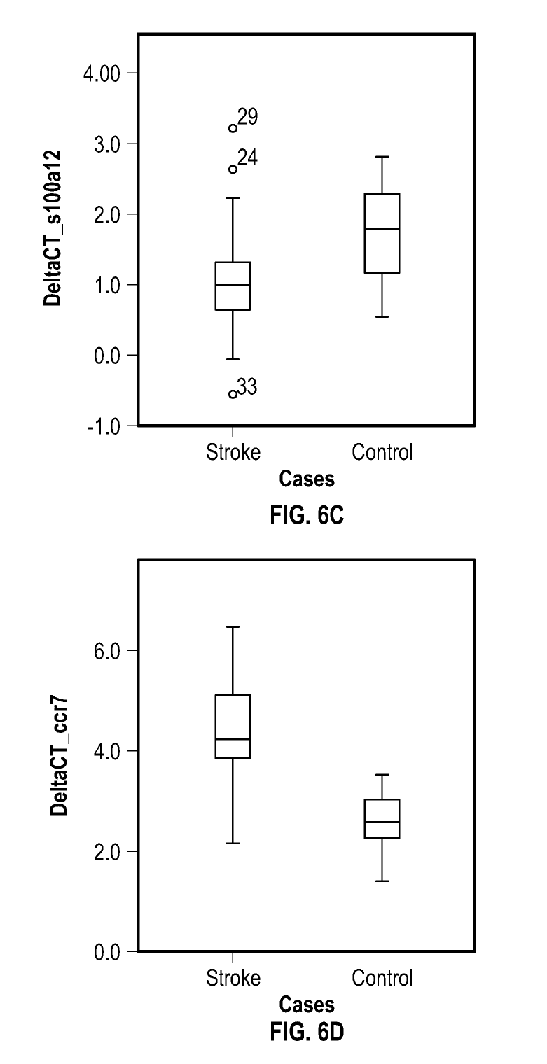

PCR was performed to measure the gene expression of ARG, MMP9, s100a12 and CCR7. The expression levels of ARG1 (p=0.003), MMP9 (p=0.001), s100a12 (p=0.018) and CCR7 (p=0.000) were significantly different among stroke versus metabolic disease controls (FIGS. 6A-6D).

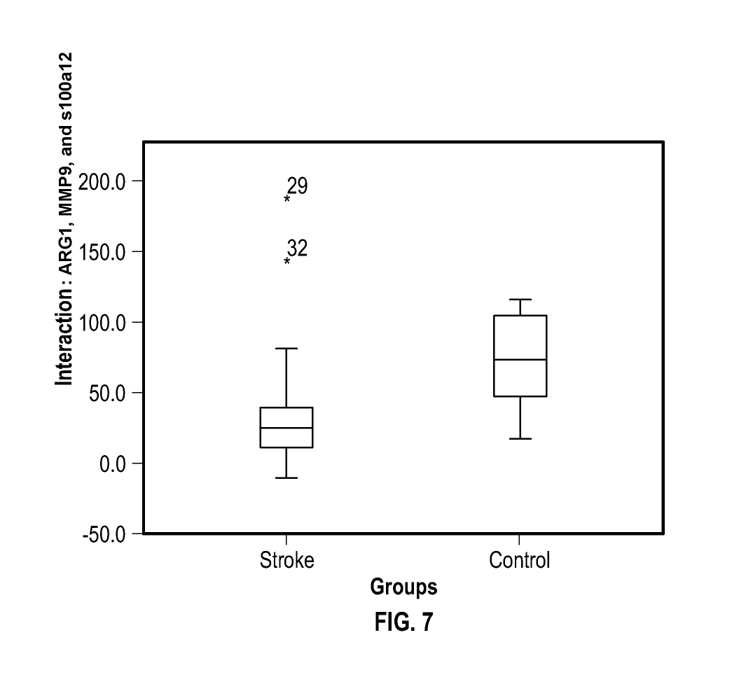

The interaction among ARG1, MMP9 and s100a12 was significantly different across the ischemic stroke group and the metabolic disease control group (p=0.009) (FIG. 7). This interaction was a pattern of expression of all the variables of interest. Pattern recognition and machine learning analyses can be performed to fully capture the patterns of expression for each disease cohort.

### Example 3—Comparison of the Protein Expression Patterns of Biomarkers Among Ischemic Stroke Patients, Transient Ischemic Attack Patients and Stroke Mimic Patients Using ELISA

Whole blood samples from three groups of patients (i.e., 4 ischemic stroke patients, 2 TIA patients, and 2 stroke mimic patients) were collected in EDTA tubes (Becton Dickinson). Plasma was removed by centrifugation.

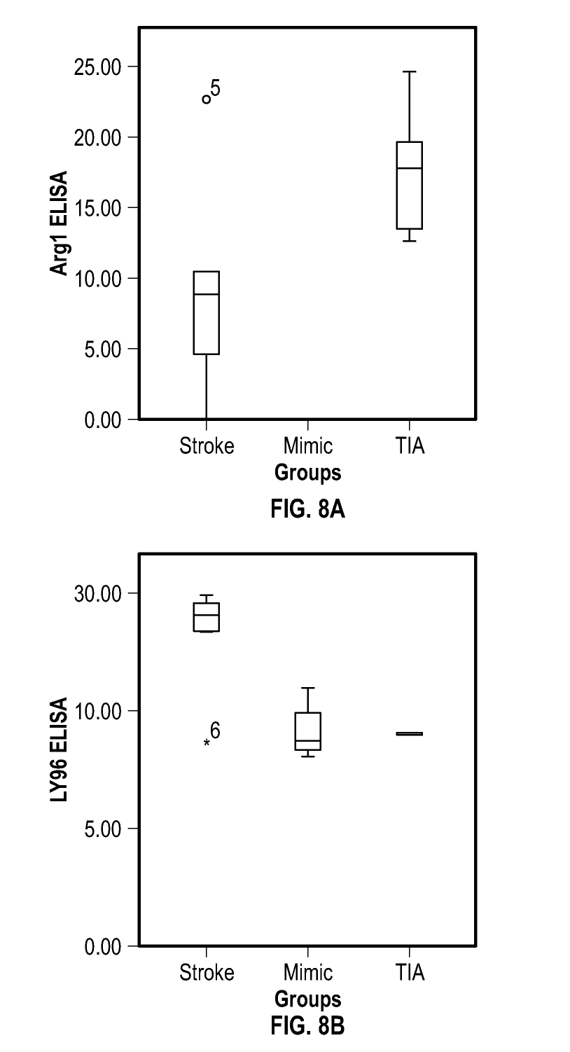

Protein expression of ARG1, CA4, CCR7, CSPG2, IQGAP1, LY96, MMP9, RAGE and ORM1 were measured using commercially available ELISA kits. The protein expression levels of ARG1 (p=0.048) and LY96 (p=0.056) were significantly different among the ischemic stroke group, the TIA group and the stroke mimic group (FIGS. 8A-8B).

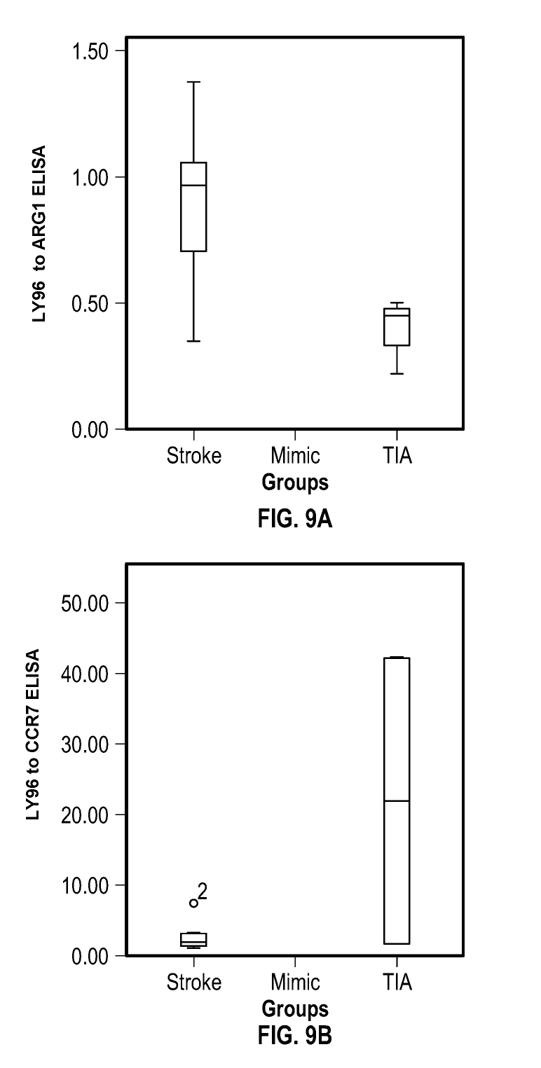

The interactions between LY96 to ARG (p=0.07) and LY96 to CCR7 (p=0.09) was significantly different among the three groups (FIGS. 9A-9B), suggesting different patterns of protein expression among the groups. The interaction was a pattern of expression of all the variables of interest. The patterns of expression for each disease cohort can be fully captured by pattern recognition and machine learning analyses.

### Example 4—Comparison of the Whole Proteomic Profile of Whole Blood Samples Between Ischemic Stroke Patients and TIA Patients

Whole blood samples from two groups of patients (i.e., 10 ischemic stroke patients and 4 TIA patients) were collected in EDTA tubes (Becton Dickinson). Plasma samples were collected from the blood samples by centrifugation. The collected plasma samples were thawed before the proteomic analysis. The proteomic analysis on the plasma samples was performed by mass spectrometry using Protea Bioscience LAESI technology. The entire proteome was screened.

Protein expression levels were compared between the ischemic stroke samples and TIA samples. FIG. 10A listed exemplary proteins that have different expression levels between the ischemic stroke group and TIA group (FIG. 10A). Pathway analysis revealed that most of these proteins were involved in coagulation. There were also significant differences between male patients and female patients (FIGS. 10A-10H).

### Example 5—Comparison of the Expression Patterns of Cytokines Between Ischemic Stroke Patients, TIA Patients, and Stroke Mimic Groups Using the Luminex System

Whole blood samples from three groups of patients (i.e., 17 ischemic stroke patients, 10 stroke mimic and 13 TIA patients) were collected in EDTA tubes (Becton Dickinson). Plasma samples were collected from the blood samples by centrifugation. The collected plasma samples were thawed before the analysis of cytokine expression patterns.

The expression levels of cytokines in the collected plasma samples were measured by a Luminex system via commercially available cytokine kits, which measure the following cytokines: BAFF, MMP9, APP, Aggrecan, Galectin-3, Fas, RAGE, Ephrin A2, CD30, TNFR1, CD27, CD40, TNFα, 116, IL8, IL10, IL1beta, IFNy, RANTES, IL1α, IL4, IL17, 112, GMCSF, ENA78, IL5, IL23P70, TARC, GroAlpha, IL33, BLCBCA, IL31, and MCP2.

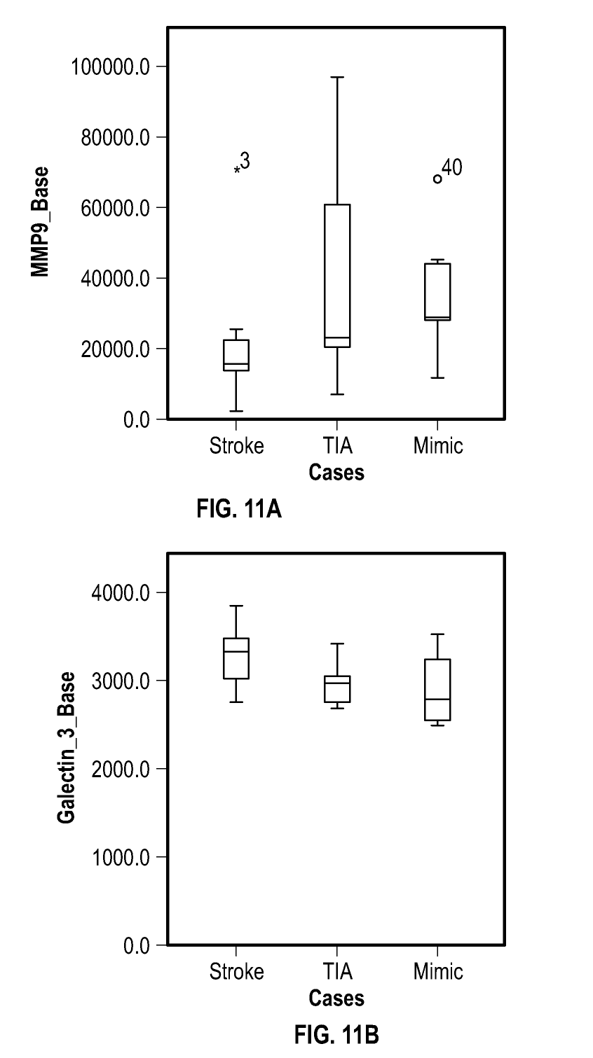

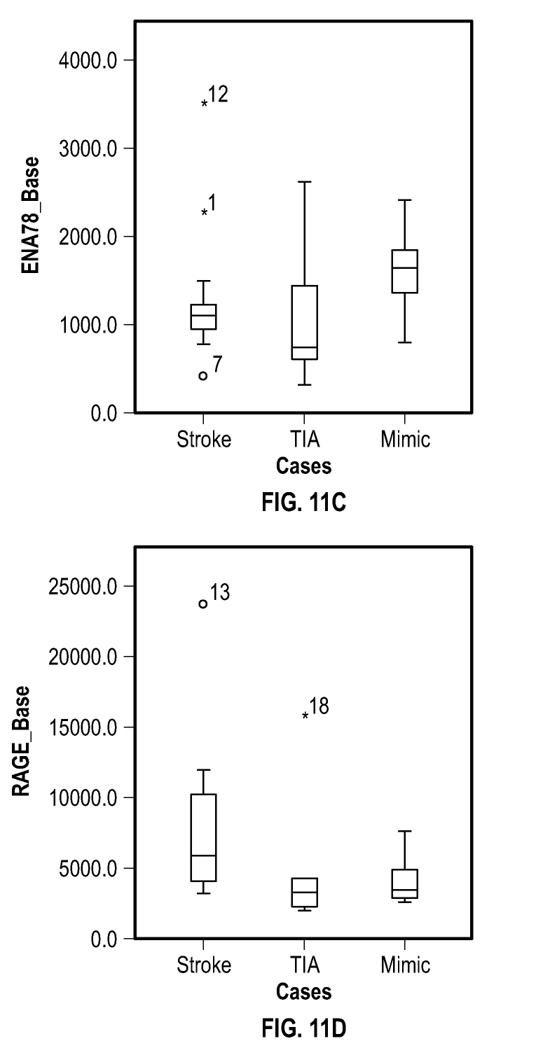

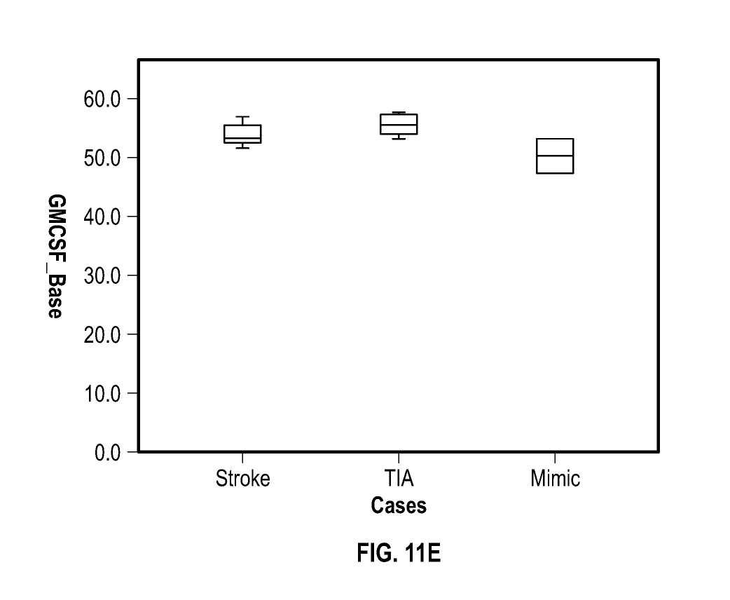

The expression levels of MMP9 (p=0.065), Galectin 3 (p=0.09), RAGE (p=0.06), CD30 (p=0.078), GMCSF (p=0.07), and ENA78 (p=0.028) were significantly different among all three groups (FIGS. 11A-11E).

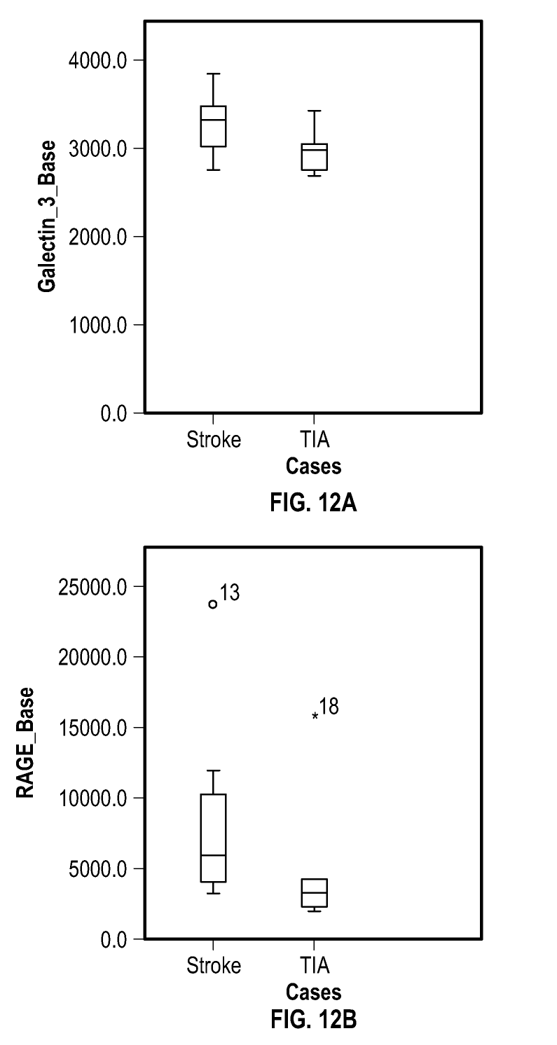

The expression levels of Galectin 3 (p=0.09) and RAGE (p=0.09) were significantly different between the ischemic stroke group and the TIA group (FIGS. 12A-12B).

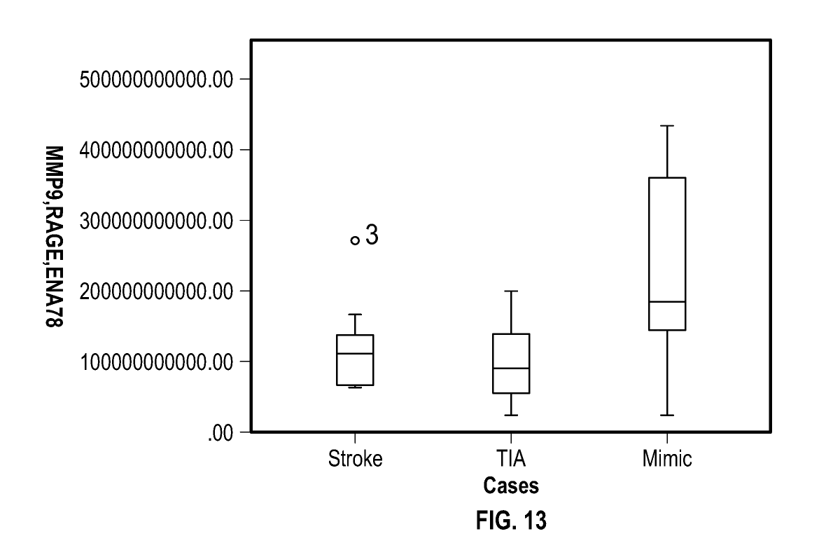

The interaction among MMP9, RAGE, and ENA78 was statistically different among all three groups (p=0.048) (FIG. 13).

### Example 6—Comparison of the Profiles of Blood Samples Among Ischemic Stroke Patients, with Non-Ischemic Stroke Patients

Whole blood samples were collected from five groups of patients (i.e., 43 ischemic stroke patients, 13 TIA patients, 3 hemorrhage stroke patients, 14 traumatic brain injury (TBI) patients, and 22 stroke mimic patients). Various tests were performed to measure the profiles of the blood samples.

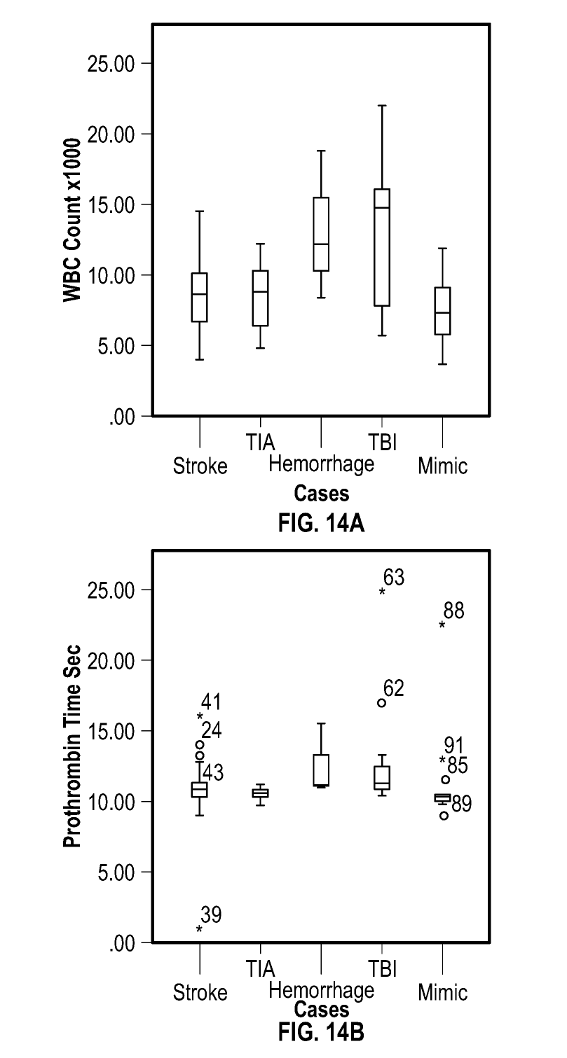

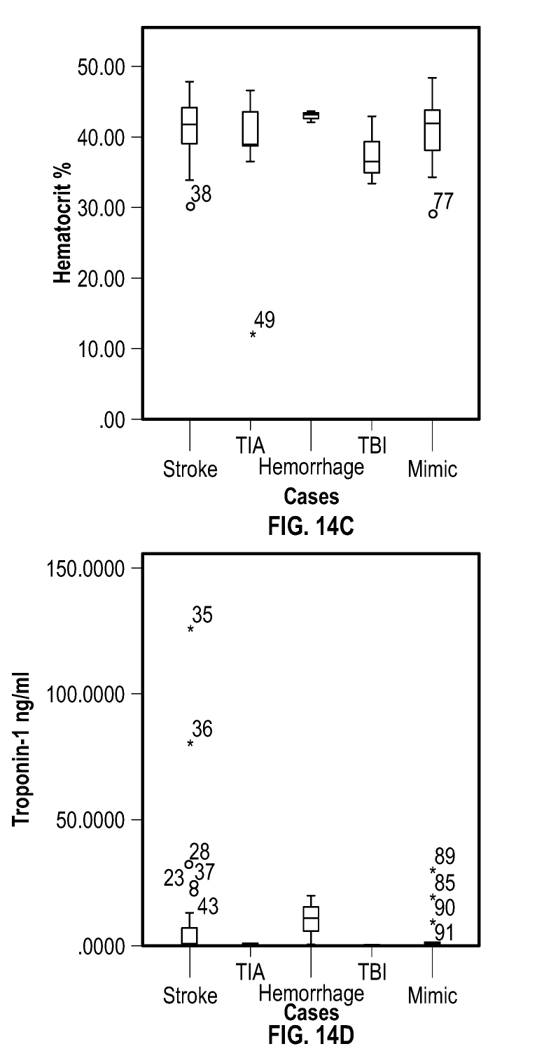

Baseline levels of total white blood cells (p=0.012), platelets count (p=0.07), hematocrit (p=0.036), hemoglobin level (p=0.1), prothrombin time (p=0.004), activated partial thromboplastin time (APTT) (p=0.028), troponin 1 (p=0.09), and creatinine kinase (p=0.07) were significantly different among all five groups (FIGS. 14A-14D).

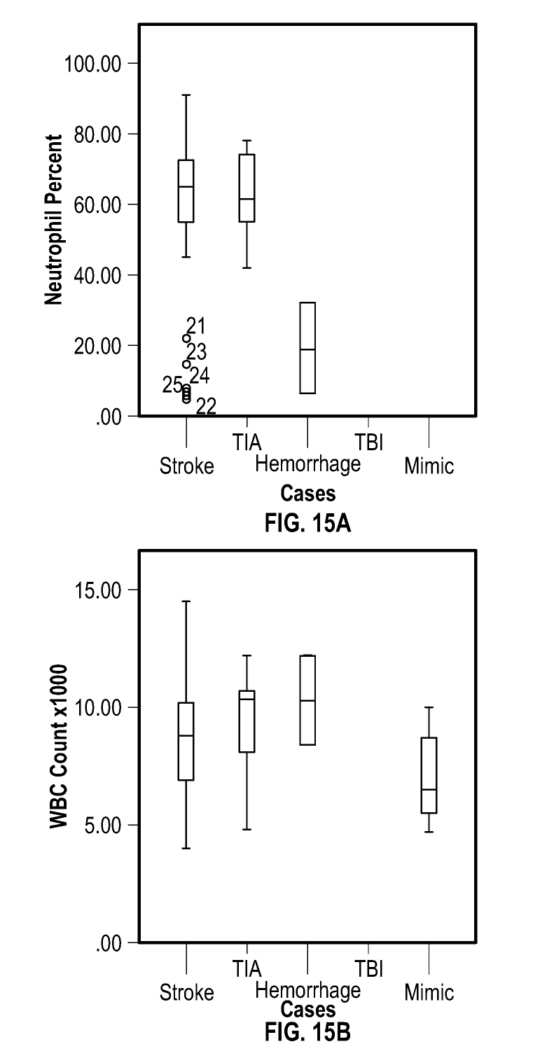

Baseline levels of total white blood cell count (p=0.011), the neutrophil percentage (p=0.05), and creatine kinase-MB (p=0.018) were significantly different among the ischemic stroke group, the TIA group and the hemorrhagic ischemic stroke group (FIGS. 15A-15B).

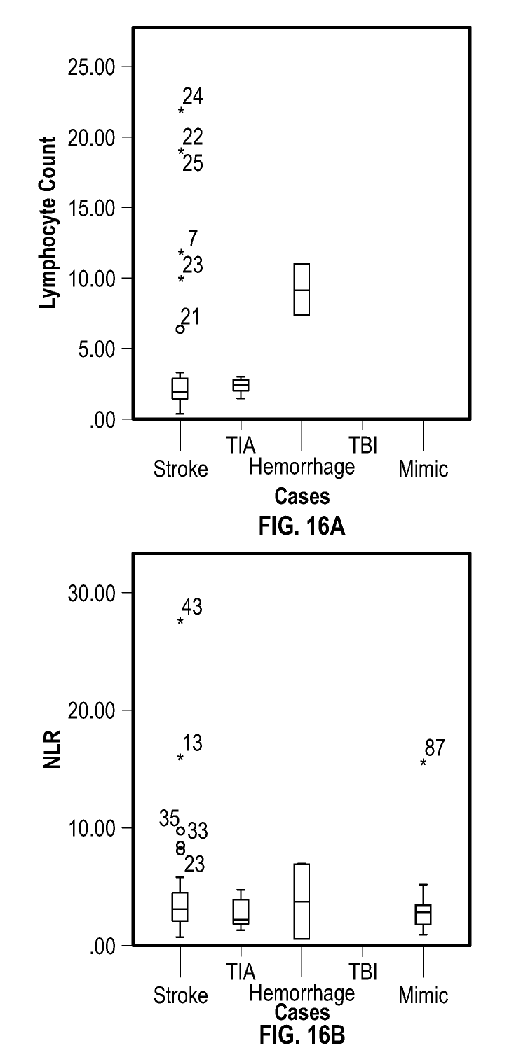

The lymphocyte count and neutrophil lymphocyte ratio were very high in the hemorrhagic stroke group. The lymphocyte count and neutrophil lymphocyte ratio were statistically different between the hemorrhagic stroke group and the TIA group, but not statistically different between the ischemic stroke group and the hemorrhagic stroke group (FIGS. 16A-16B).

### Example 7—Correlations Between Time from Ischemic Stroke Symptom Onset and Biomarkers at Select Time Points

Whole blood RNA was extracted from paxgene tubes per paxgene protocol. Whole genome expression profiling was determined via Illumina human ref8 v2 bead chips. Blood was drawn at two time points (0-24 from stroke onset and again 24-48 hours later). Relationships between gene expression and time from symptom onset were determined using the Pearson correlation. Differences between baseline and follow up were determined by paired samples t-test. Genes in innate and adaptive immune pathways were targeted. Toll like receptor (TLR) genes TLR2, TLR4, LY96, MYD88, JAK2; Cytotoxic T lymphocyte Antigen-4 (CTLA4) genes CD3, CD4, SYK; Genomic markers in other immune pathways (AKAP7, CEBPB, IL10, IL8, IL22R; and Genomic markers from the diagnostic panel (ARG1, CA4, CCR7). N=34 ischemic stroke subjects.

Correlations between time from symptom onset (from 0-48 hours) and target genes at each time point: Toll like receptor (TLR) genes TLR2 (0.1), TLR4 (0.3), LY96 (0.000), MYD88 (0.000), JAK2 (0.006). Cytotoxic T lymphocyte Antigen-4 (CTLA4) genes CD3 (0.002), CD4 (0.006), SYK (0.001) Genomic markers in other immune pathways (AKAP7 (0.002), CEBPB (0.000), IL10 (0.000), IL8 (0.003), IL22R (0.001) Genomic markers from diagnostic panel (ARG1 (0.1), CA4 (0.3), CCR7 (0.1).

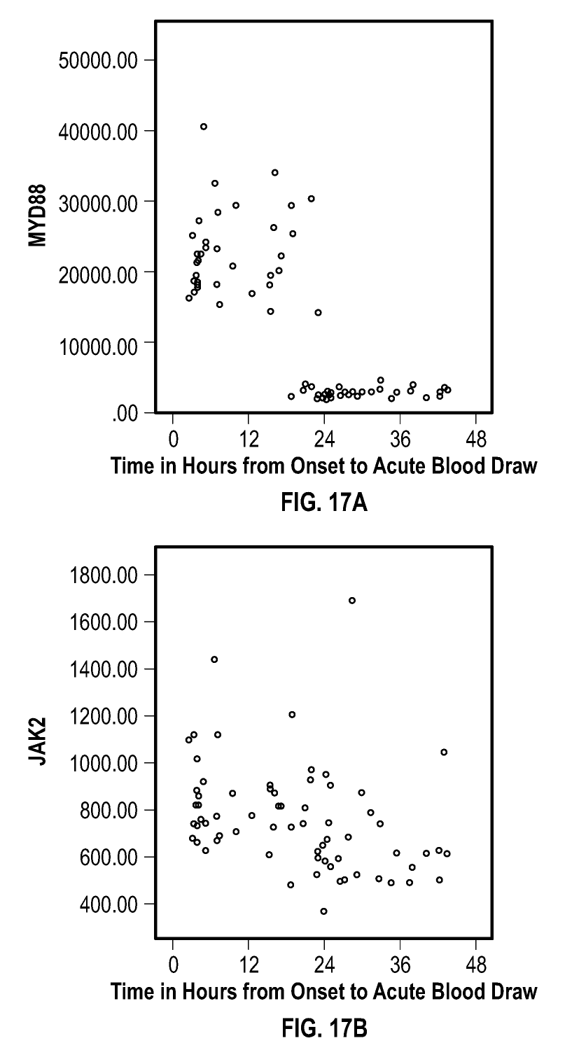

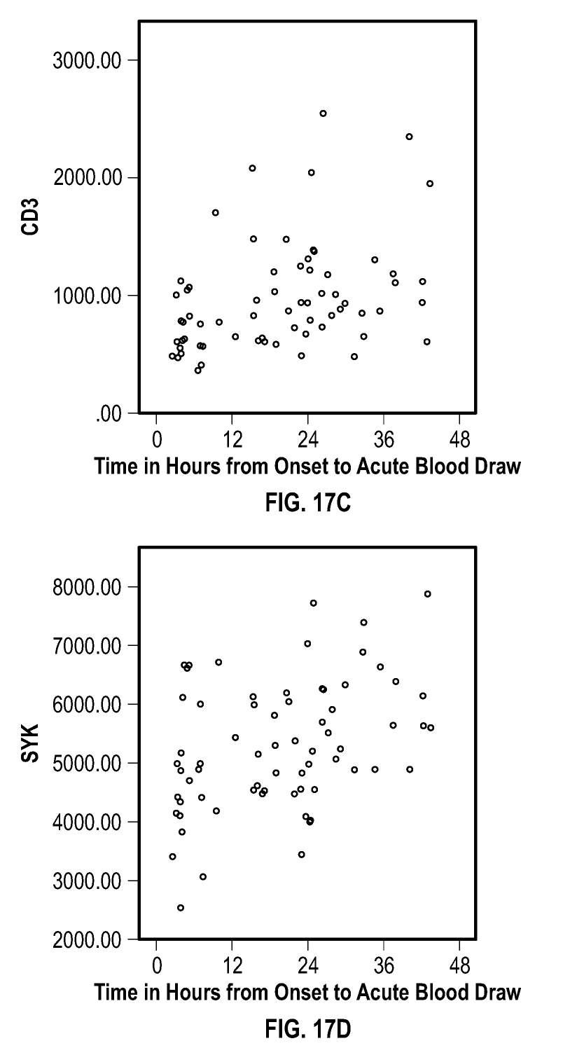

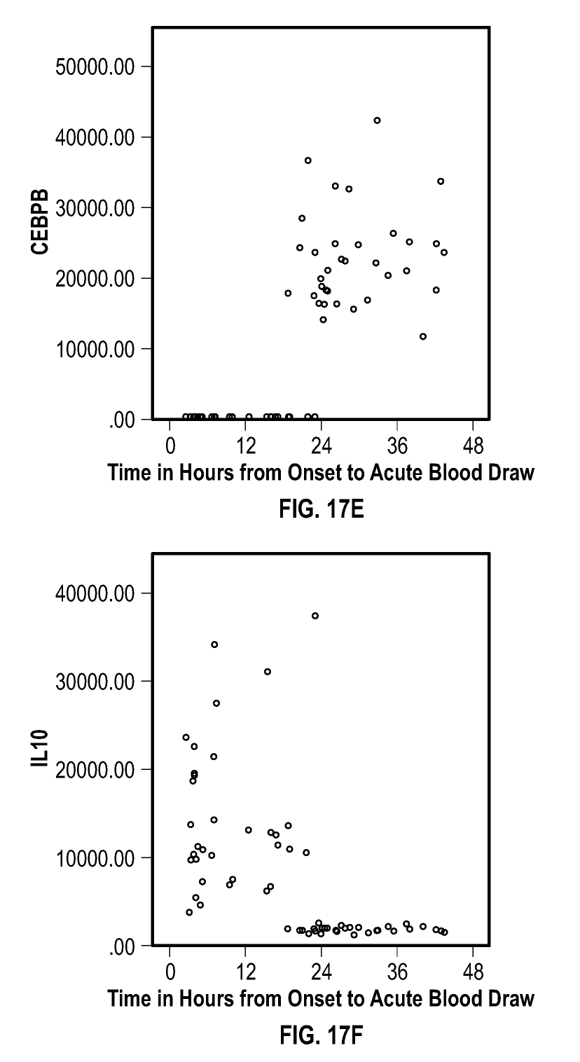

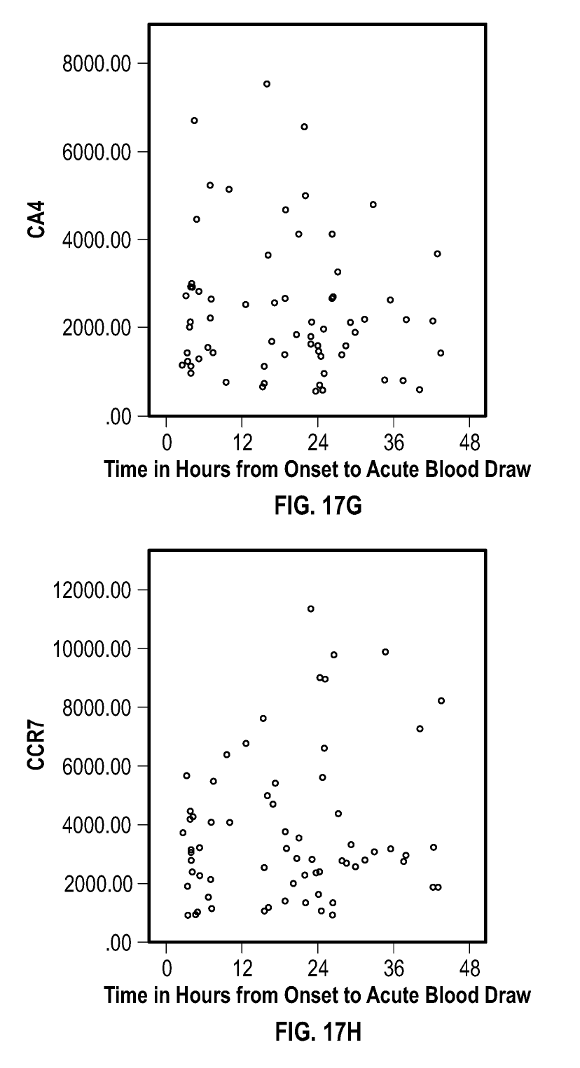

Paired samples t-test for differences between baseline (0-24 hours) and follow up (24-48 hours): Toll like receptor (TLR) genes TLR2 (0.013), TLR4 (0.08), LY96 (0.000), MYD88 (0.000), JAK2 (0.001). Cytotoxic T lymphocyte Antigen-4 (CTLA4) genes CD3 (0.002), CD4 (0.001), SYK (0.001) Genomic markers in other immune pathways (AKAP7 (0.000), CEBPB (0.000), IL10 (0.000), IL8 (0.000), IL22R (0.002). Genomic markers from our diagnostic panel (ARG1 (0.03), CA4 (0.03), CCR7 (0.1). (FIGS. 17A-17H)

### Example 8—Correlations Between Time of Ischemic Stroke Symptom Onset and Select Biomarkers

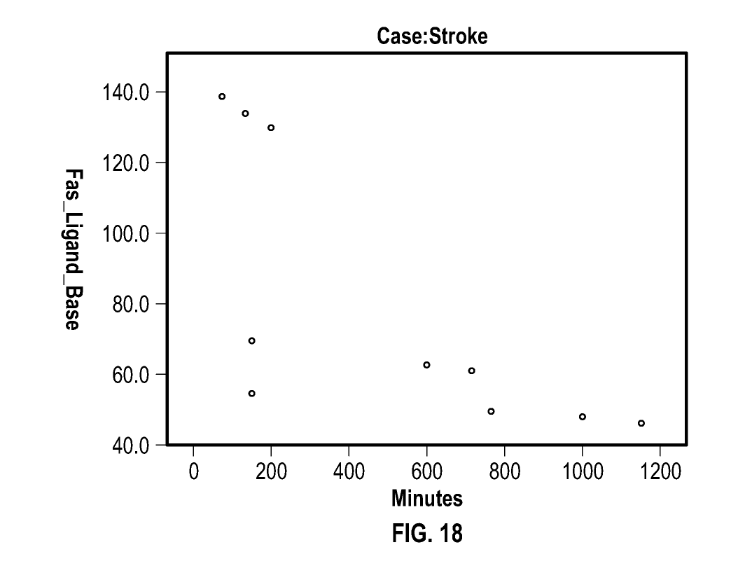

Plasma was separated from whole blood obtained in EDTA tubes via centrifugation, frozen at −80° C. and thawed for analysis on the Luminex system via commercially available cytokine kits. Blood was collected at one time point (0-24 hours from onset of symptoms). N=17 ischemic stroke subjects. The following cytokines were included in the analysis (FAS ligand, IL6, and 1110). (FIG. 18)

Pearson correlation between time from symptom onset and expression of selected markers: Fas ligand r=−0.71; p=0.021, IL6 r=−0.68; p=0.1, IL10 r=−0.5; p=0.3

### Example 9—Correlation Between Time of Ischemic Stroke Symptom Onset and Proteomic Markers

Plasma was separated from whole blood obtained in EDTA tubes via centrifugation, frozen at −80° C. and thawed for analysis via Protea Biosciences LAESI technology (mass spectrometry) which screens the entire proteome. Blood was collected at one time point (0-24 hours from onset of symptoms). N=10 ischemic stroke subjects. Immunoglobulin gamma 3 (IGG3), Isoform 2 of Teneurin1, Immunoglobulin gamma 4 (IGG4), and Isoform 2 of aDisintegrin, were correlated with time from stroke symptom onset.

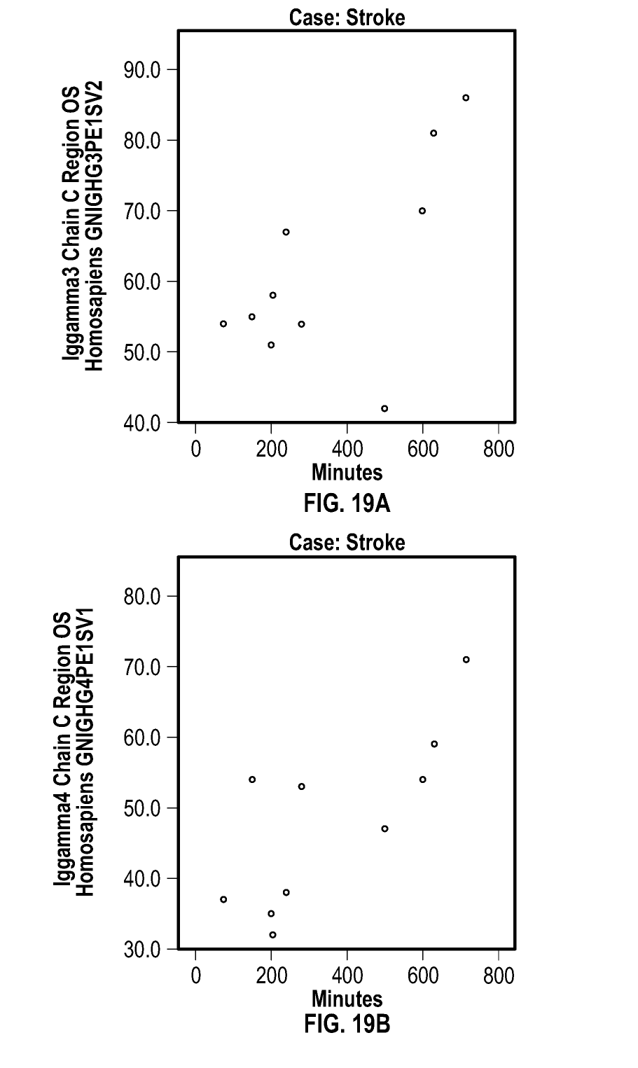

Pearson correlation between time from symptom onset and expression of selected markers: Immunoglobulin gamma 3 (IGG3) (r=0.6; p=0.04), Isoform 2 of Teneurin1 (r=−0.9; p=0.008), Immunoglobulin gamma 4 (IGG4) (r=0.8; p=0.01), and Isoform 2 of aDisintegrin (r=0.9; p=0.07), were correlated with time from stroke symptom onset. (FIGS. 19A-19B).

### Example 10—Correlation Between Time of Ischemic Stroke Symptom Onset and Immune Markers

Creatine kinase MD (CKMB) and platelet counts were obtained via the acute blood draw from medical record. Blood was collected at one time point (0-24 hours from onset of symptoms). N=17 ischemic stroke subjects.

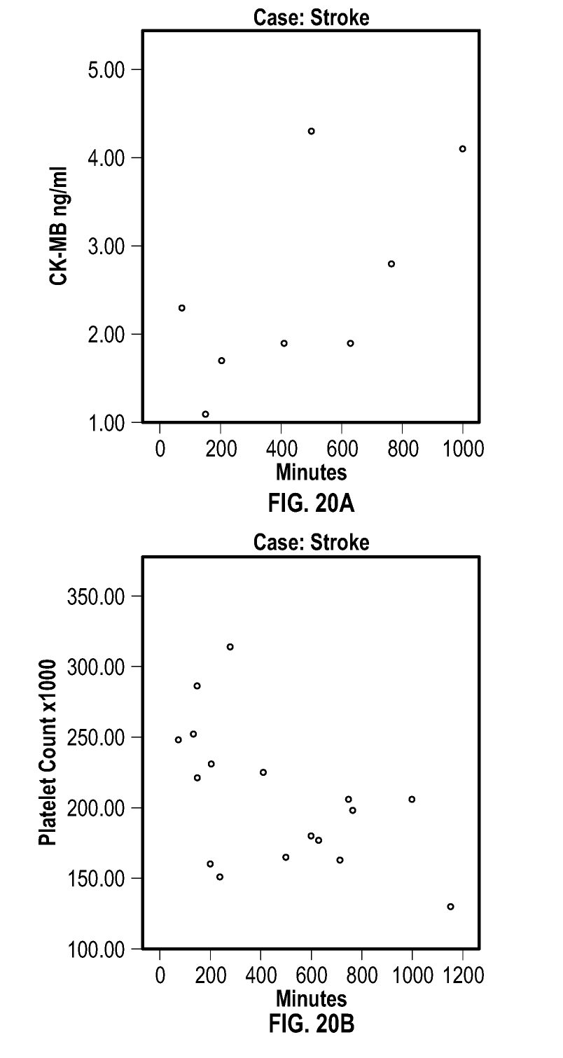

Pearson correlation between time from symptom onset and expression of selected biomarkers: Platelet count: r=−0.5; p=0.026, CK-mb: r=0.6; p=0.08. (FIGS. 20A-20B).

### Example 11—Machine Learning Approach Identified a Pattern of Gene Expression in Peripheral Blood Capable of Identifying Acute Ischemic Stroke with High Levels of Accuracy

A two-stage study design was used which included a discovery cohort and an independent validation cohort. In the discovery cohort, peripheral whole blood samples were obtained from 39 AIS patients upon emergency department admission, as well as from 24 neurologically asymptomatic controls. Microarray was used to measure the expression levels of over 22,000 genes and GA/kNN was used to identify a pattern of gene expression which optimally discriminated between AIS patients and controls. Then, in a separate validation cohort, the gene expression pattern identified in the discovery cohort was evaluated for its ability to discriminate between 39 AIS patients and each of two different control groups, one consisting of 30 neurologically asymptomatic controls, and the other consisting of 15 stroke mimics, with gene expression levels being assessed by qRT-PCR.

### Discovery Cohort:

Acute ischemic stroke patients and neurologically asymptomatic controls were recruited from 2007 to 2008 at Suburban Hospital, Bethesda, Md. For AIS patients, diagnosis was confirmed by MRI and all samples were collected within 24 hours of symptom onset, as determined by the time the patient was last known to be free of AIS symptoms. Injury severity was determined according to the NIH stroke scale (NUBS) at the time of blood draw. Control subjects were deemed neurologically normal by a trained neurologist at the time of enrolment. Demographic information was collected from either the subject or significant other by a trained clinician. All procedures were approved by the institutional review boards of the National Institute of Neurological Disorders/National Institute on Aging at NIH and Suburban Hospital. Written informed consent was obtained from all subjects or their authorized representatives prior to any study procedures.

### Blood Collection and RNA Extraction:

Peripheral whole blood samples were collected via PAXgene RNA tubes (Qiagen, Valencia, Calif.) and stored at −80° C. until RNA extraction. Total RNA was extracted via PreAnalytiX PAXgene blood RNA kit (Qiagen) and automated using the QIAcube system (Qiagen). Quantity and purity of isolated RNA was determined via spectrophotometry (NanoDrop, Thermo Scientific, Waltham, Mass.). Quality of RNA was confirmed by chip capillary electrophoresis (Agilent 2100 Bioanalyzer, Agilent Technologies, Santa Clara, Calif.).

### RNA Amplification and Microarray:

RNA was amplified and biotinylated using the TotalPrep RNA amplification kit (Applied Biosystems, Grand Island, N.Y.). Samples were hybridized to HumanRef-8 expression bead chips (Illumina, San Diego, Calif.) containing probes for transcripts originating from over 22,000 genes and scanned using the Illumina BeadStation. Raw probe intensities were background subtracted, quantile normalized, and then summarized at the gene level using Illumina GenomeStudio. Sample labeling, hybridization, and scanning were performed per standard Illumina protocols.

### GA/kNN Analysis:

Normalized microarray data were filtered based on absolute fold difference between stroke and control regardless of statistical significance; genes exhibiting a greater than 1.7 absolute fold difference in expression between AIS and control were retained for analysis. Filtered gene expression data were z-transformed and GA/kNN analysis was performed using source code developed by Li et al., Gene assessment and sample classification for gene expression data using a genetic algorithm/k-nearest neighbor method. Comb Chem High Throughput Screen. 2001; 4(8):727-739. Two-thousand near-optimal solutions were collected per sample using five nearest neighbors, majority rule, a chromosome length of 5, and a termination cutoff of 0.97. Leave one out cross validation was performed using the top 50 ranked gene products.

### Validation Cohort:

AIS patients, stroke mimics, and neurologically asymptomatic controls were recruited from 2011 to 2015 at Ruby Memorial Hospital, Morgantown, W. Va. As with the discovery cohort, AIS diagnosis was confirmed via neuroradiological imaging and blood was sampled within 24 hours of known symptom onset. Patients admitted to the emergency department with stroke-like systems but receiving a negative diagnosis for stroke upon imaging were identified as stroke mimics. Assessment of injury severity, screening of neurologically asymptomatic controls, and collection of demographic information was performed in an identical manner as it was with the discovery cohort. All procedures were approved by the institutional review boards of West Virginia University and Ruby Memorial Hospital. Written informed consent was obtained from all subjects or their authorized representatives prior to study procedures.

### Quantitative Reverse Transcription PCR:

cDNA was generated from purified RNA using the Applied Biosystems high capacity reverse transcription kit. For qPCR, target sequences were amplified from 10 ng of cDNA input using sequence specific primers (Table 2) and detected via SYBR green (PowerSYBR, Thermo-Fisher) on the RotorGeneQ (Qiagen). Raw amplification plots were background corrected and CT values were generated via the RotorGeneQ software package. All reactions were performed in triplicate. B2M, PPIB, and ACTB were amplified as low variability reference transcripts and normalization was performed using the NORMA-gene data-driven normalization algorithm.¹³ All expression values were presented as fold difference relative to control.

TABLE 2
Primers and thermocycling conditions used for qRT-PCR.

| Gene | Transcripts¹ | Primers (5′ TO 3′)² | | Product (bp) |
|---|---|---|---|---|
| ANTXR2 | NM_058172.5 | FOR: | GATCTCTACTTCGTCCTGGACA | 90 |
| | NM_001145794.1 | REV: | AAATCTCTCCGCAAGTTGCTG | |
| STK3 | NM_006281.3 | FOR: | CGATGTTGGAATCCGACTTGG | 105 |
| | XM_011517258.1 | REV: | GTCTTTGTACTTGTGGTGAGGTT | |
| | XM_011517255.1 | | | |
| | XM_011517254.1 | | | |
| | XM_011517253.1 | | | |
| | XM_011517252.1 | | | |
| | XM_011517251.1 | | | |
| | XM_011517250.1 | | | |
| | XM_011517249.1 | | | |
| | XM_011517247.1 | | | |
| | NM_001256312.1 | | | |
| | NM_001256313.1 | | | |
| PDK4 | NM_002612.3 | FOR: | GACCCAGTCACCAATCAAAATCT | 82 |
| | | REV: | GGTTCATCAGCATCCGAGTAGA | |
| CD163 | NM_004244.5 | FOR: | GCGGGAGAGTGGAAGTGAAAG | 89 |
| | XM_005253529.3 | REV: | GTTACAAATCACAGAGACCGCT | |
| | XM_005253528.3 | | | |
| | NM_203416.3 | | | |
| MAL | NM_002371.3 | FOR: | GCCCTCTTTTACCTCAGCG | 95 |
| | NM_022439.2 | REV: | GCAATGTTTTCATGGTAGTGCCT | |
| GRAP | NM_006613.3 | FOR: | AGCCCTTGCTCAAGTCACC | 180 |
| | | REV: | CGTAACTCCGTGGGAAGAAGC | |
| ID3 | NM_002167.4 | FOR: | GAGAGGCACTCAGCTTAGCC | 170 |
| | | REV: | TCCTTTTGTCGTTGGAGATGAC | |
| CTSZ | NM_001336.3 | FOR: | CAGCGGATCTGCCCAAGAG | 198 |
| | | REV: | CGATGACGTTCTGCACGGA | |
| PLXDC2 | NM_032812.8 | FOR: | ACTCAGATCGAGGAGGATACAGA | 75 |
| | XM_011519750.1 | REV: | CCGGCTGGCAGAATCAGATG | |
| KIF1B | NM_015074.3 | FOR: | AAACAAGGGTAATTTGCGTGTGC | 78 |
| | NM_183416.3 | REV: | GTAACTGCCAACTTGGACAGAT | |
| PPIB | NM_000942.4 | FOR: | AAGTCACCGTCAAGGTGTATTTT | 153 |
| | | REV: | TGCTGTTTTTGTAGCCAAATCCT | |
| B2M | NM_004048.2 | FOR: | GAGGCTATCCAGCGTACTCCA | 248 |
| | XM_006725182.2 | REV: | CGGCAGGCATACTCATCTTTT | |
| | XM_005254549.2 | | | |
| ACTB | NM_001101.3 | FOR: | CATGTACGTTGCTATCCAGGC | 250 |
| | XM_006715764.1 | REV: | CTCCTTAATGTCACGCACGAT | |

¹Listed by NCBI accession number
²All targets were amplified for 40 cycles of 95° C. (15s)/60° C. (60s)

### Statistical Analysis:

Statistical analysis was performed using the SPSS statistical software package (IBM, Chicago, Ill.). Chi squared analysis was used for comparison of dichotomous variables while student t-test was used for the comparison of continuous variables. In the case of multiple t-tests, the Benjamin Hochberg correction was applied to p-values using a false discovery rate cutoff of 5%. The level of significance was established at 0.05 for all statistical testing.

### Results

### Discovery Cohort:

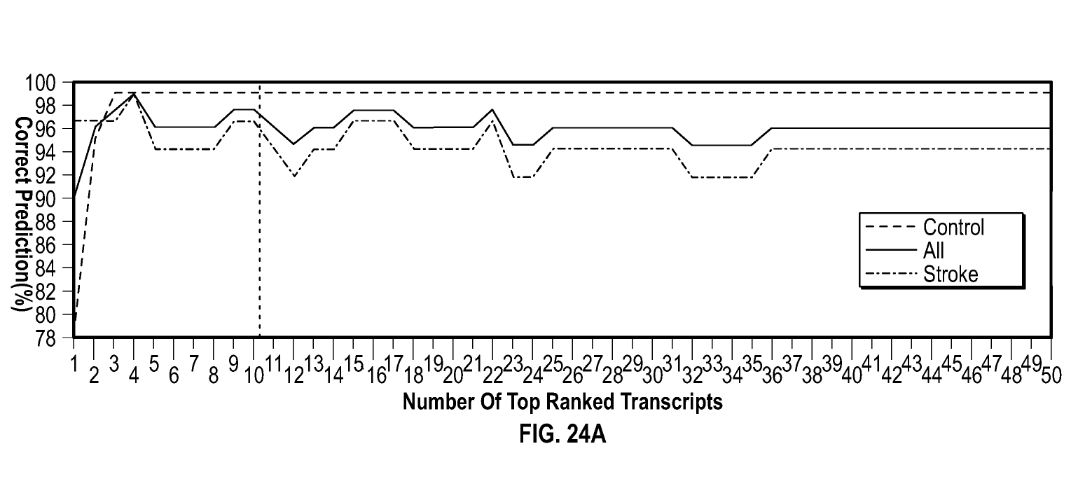

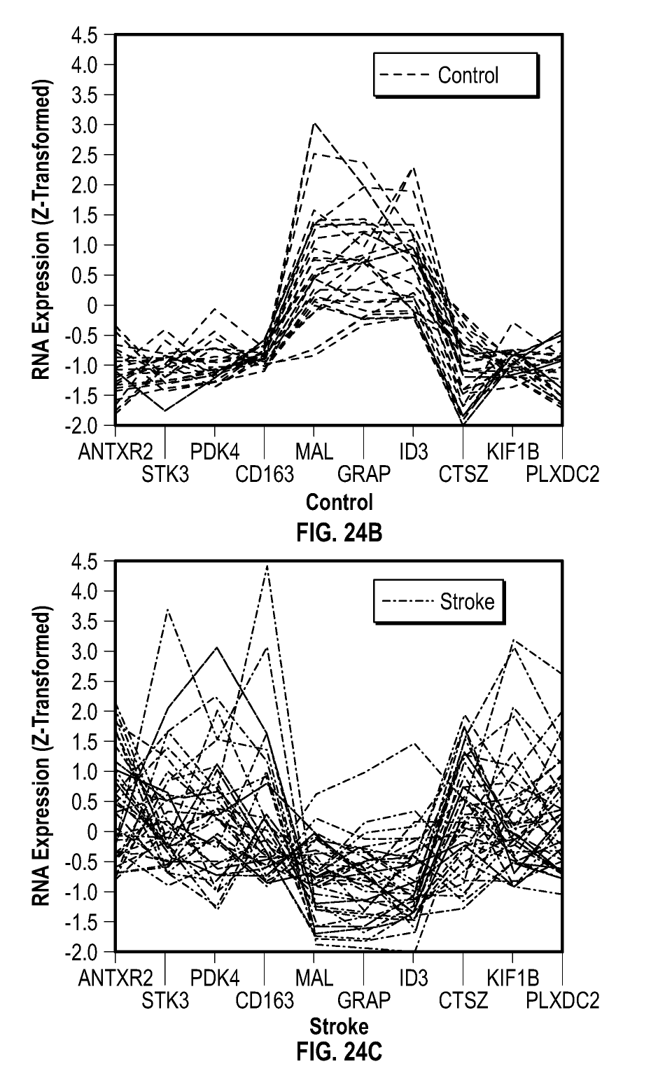

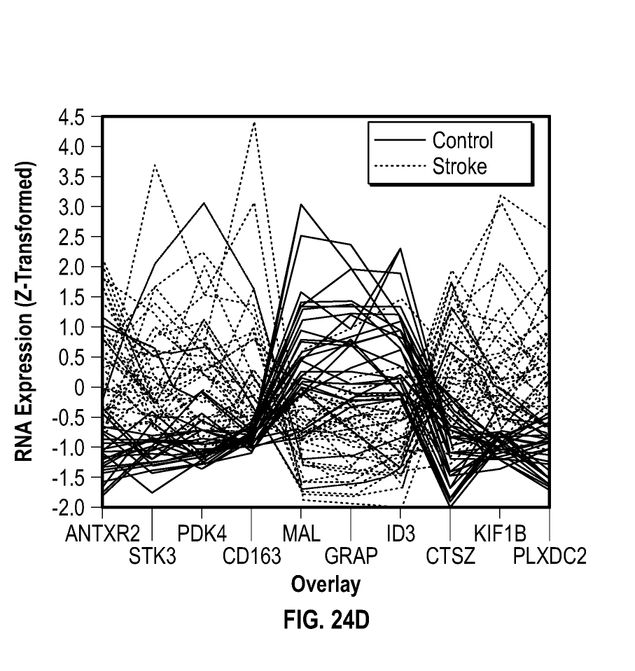

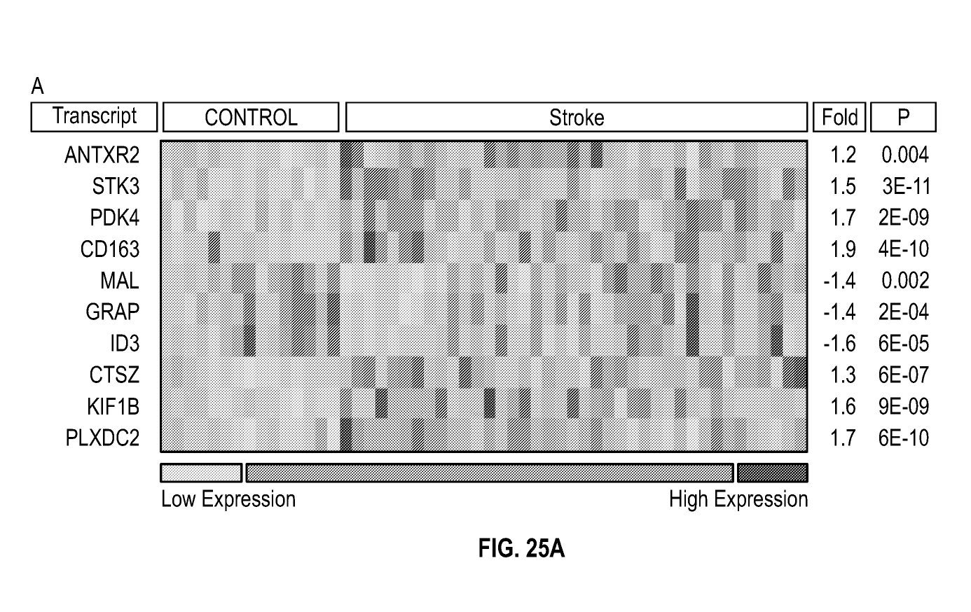

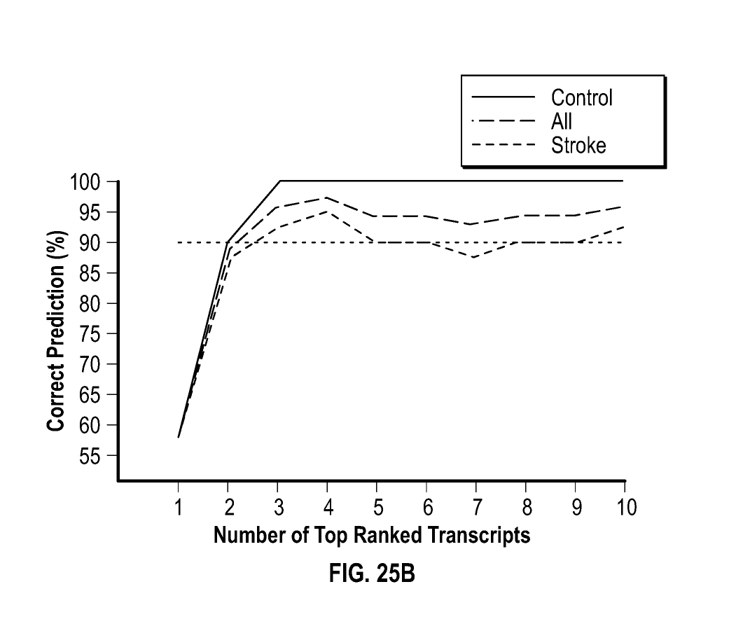

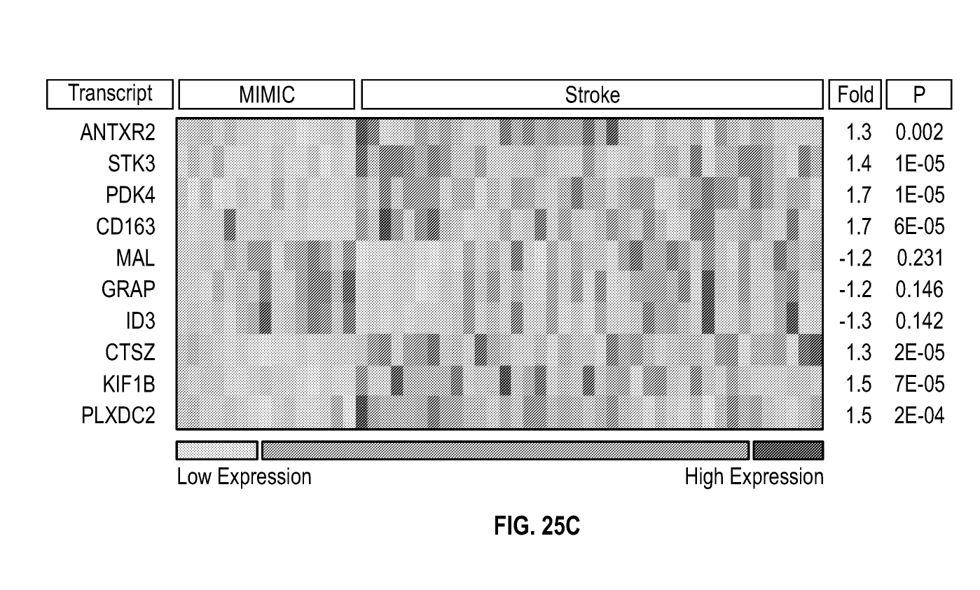

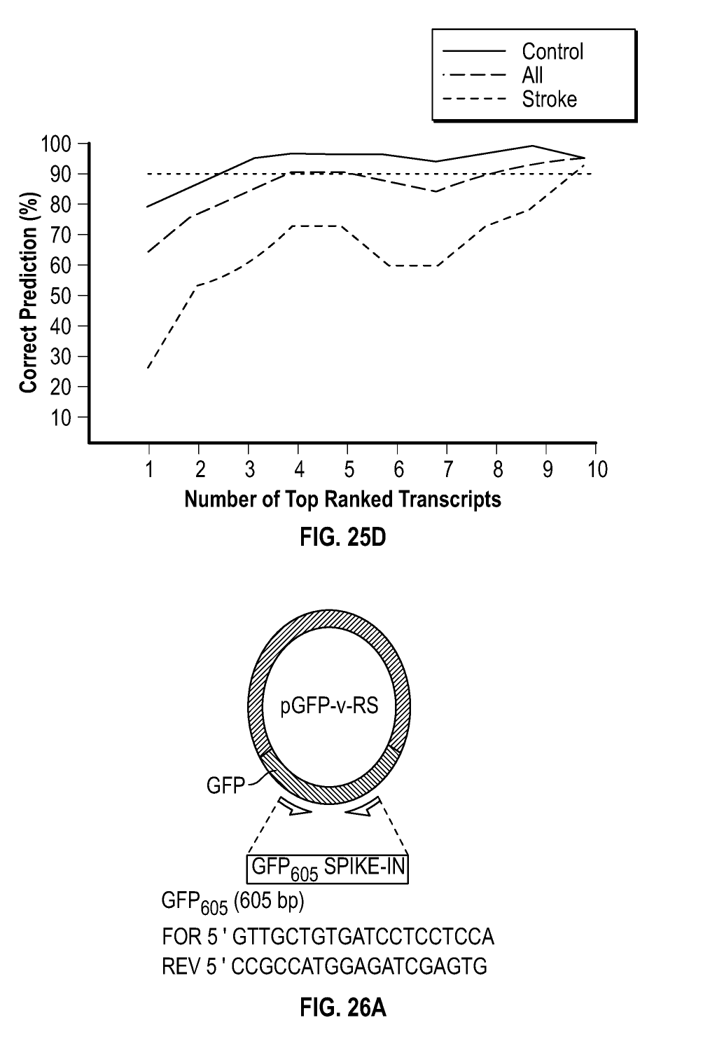

In terms of demographic and clinical characteristics, AIS patients were significantly older than neurologically asymptomatic controls and displayed a higher prevalence of co-morbidities such as hypertension and dyslipidemia (Table 3). The top 50 peripheral blood transcripts ranked by GA/kNN based on their ability to discriminate between AIS patients and controls are depicted in FIG. 23A, ordered by the number of times each transcript was selected as part of a near-optimal solution. Differential peripheral blood expression of the top 50 transcripts between AIS patients and controls are presented in FIG. 23B. The top 50 transcripts identified by GA/kNN displayed a strong ability to discriminate between AIS patients and controls using kNN in leave one out cross validation; a combination of just the top 10 ranking transcripts (ANTXR2, STK3, PDK4, CD163, MAL, GRAP, ID3, CTSZ, KIF1B, and PLXDC2) were able to identify 98.4% of subjects in the discovery cohort correctly with a sensitivity of 97.4% and specificity of 100% (FIGS. 24A and 24B). The combined discriminatory power of the top 10 transcripts was evident when their expression levels were plotted for each individual subject; the overall pattern of expression was different between AIS patients and controls (FIGS. 25B, 25C, and 25D).

TABLE 3
Discovery cohort clinical characteristics

| | ASYMPTOMATIC CONTOL vs STROKE | | | |
|---|---|---|---|---|
| | CONTROL | STROKE | | |
| | (n = 24) | (n = 39) | STAT (df) | p |
| Age (mean ± SD) | 59.9 ± 9.7 | 73.1 ± 14.0 | t = −4.40 (61) | 0.000 * |
| Male n (%) | 9 (41.7) | 17 (43.6) | χ² = 0.12 (1) | 0.731 |
| Female n (%) | 14 (58.3) | 22 (56.4) | χ² = 0.12 (1) | 0.731 |
| NIHSS (mean ± SD) | 0 ± 0.0 | 5.3 ± 6.4 | t = 5.17 (38) | 0.000 * |
| Family history of stroke n (%) | 4 (16.7) | 15 (38.5) | χ² = 7.02 (1) | 0.008 * |
| Hypertension n (%) | 7 (29.2) | 25 (64.1) | χ² = 11.2 (1) | 0.001 * |
| Dyslipidemia n (%) | 0 (0.00) | 18 (46.2) | χ² = 15.5 (1) | 0.000 * |
| Diabetes n (%) | 2 (8.3) | 11 (28.2) | χ² = 3.58 (1) | 0.058 |
| Previous stroke n (%) | 2 (8.30) | 6 (15.4) | χ² = 0.67 (1) | 0.414 |
| Atrial fibrillation n (%) | 0 (0.00) | 6 (15.4) | χ² = 4.08 (1) | 0.043 |
| Myocardial infarction n (%) | 0 (0.00) | 6 (15.4) | χ² = 4.08 (1) | 0.043 |
| Hypertension medication n (%) | 8 (33.3) | 29 (74.4) | χ² = 10.3 (1) | 0.001 * |
| Diabetes medication n (%) | 1 (4.20) | 7 (17.9) | χ² = 2.55 (1) | 0.111 |
| Cholesterol medication n (%) | 5 (20.8) | 17 (43.6) | χ² = 3.39 (1) | 0.066 |
| Anticoagulant or antiplatelet n (%) | 1 (4.20) | 20 (51.3) | χ² = 14.9 (1) | 0.000 * |
| rtPA n (%) | 0 (0.00) | 9 (23.1) | χ² = 6.46 (1) | 0.011 |
| Current smoker n (%) | 2 (8.30) | 2 (5.13) | χ² = 0.26 (1) | 0.612 |

### Validation Cohort:

Like in the discovery cohort, AIS patients were significantly older than neurologically asymptomatic controls, however, AIS patients and asymptomatic controls were better matched in terms of the prevalence of co-morbidities (Table 4). AIS patients were also significantly older than stroke mimics, however, well matched with stroke mimics in terms of the presence of co-morbidities (Table 4).

TABLE 4
Validation cohort clinical characteristics

| | Asymptomatic Control v Stroke | | | | Mimic v Stroke | | | |
|---|---|---|---|---|---|---|---|---|
| | Control | Stroke | Stat | | Mimic | Stroke | Stat | |
| | (n = 30) | (n = 39) | (df) | p | (n = 15) | (n = 39) | (df) | p |
| Age (mean ± SD) | 51.5 ± 14.3 | 73.1 ± 13.3 | t = −6.41 | 0.000 * | 60.2 ± 17.2 | 73.1 ± 13.3 | t = −2.94 | 0.005 * |
| | | | (67) | | | | (52) | |
| Male n (%) | 5 (16.7) | 14 (35.9) | χ² = 3.14 | 0.076 | 7 (46.7) | 14 (35.9) | χ² = 0.53 | 0.467 |
| | | | (1) | | | | (1) | |
| Female n (%) | 25 (83.3) | 25 (64.1) | χ² = 3.14 | 0.076 | 8 (53.3) | 25 (64.1) | χ² = 0.53 | 0.467 |
| | | | (1) | | | | (1) | |
| NIHSS | 0.0 ± 0.0 | 8.6 ± 7.5 | t = 7.16 | 0.000 * | 5.0 ± 4.5 | 8.6 ± 7.5 | t = −1.74 | 0.088 |
| (mean ± SD) | | | (38) | | | | (52) | |
| Family history of | 16 (53.3) | 15 (38.5) | χ² = 1.52 | 0.213 | 4 (26.7) | 15 (38.5) | χ² = 0.66 | 0.416 |
| stroke n (%) | | | (1) | | | | (1) | |
| Hypertension n (%) | 17 (56.7) | 32 (82.1) | χ² = 5.31 | 0.021 * | 13 (86.7) | 32 (82.1) | χ² = 0.16 | 0.684 |
| | | | (1) | | | | (1) | |
| Dyslipidemia n (%) | 11 (36.7) | 16 (41.0) | χ² = 0.14 | 0.713 | 10 (66.7) | 16 (41.0) | χ² = 2.85 | 0.091 |
| | | | (1) | | | | (1) | |
| Diabetes n (%) | 2 (6.70) | 8 (20.5) | χ² = 2.62 | 0.105 | 5 (33.3) | 8 (20.5) | χ² = 0.97 | 0.324 |
| | | | (1) | | | | (1) | |
| Previous stroke | 1 (3.30) | 7 (17.9) | χ² = 3.53 | 0.061 | 4 (26.7) | 7 (17.9) | χ² = 0.51 | 0.476 |
| n (%) | | | (1) | | | | (1) | |
| Atrial fibrillation | 0 (0.00) | 13 (33.3) | χ² = 12.3 | 0.000 * | 3 (20.0) | 13 (33.3) | χ² = 0.92 | 0.337 |
| n (%) | | | (1) | | | | (1) | |
| Myocardial infarction | 0 (0.00) | 11 (28.2) | χ² = 10.0 | 0.002 * | 5 (33.3) | 11 (28.2) | χ² = 0.14 | 0.712 |
| n (%) | | | (1) | | | | (1) | |
| Hypertension medication | 15 (50.0) | 27 (69.2) | χ² = 2.63 | 0.105 | 13 (86.7) | 27 (69.2) | χ² = 1.72 | 0.191 |
| n (%) | | | (1) | | | | (1) | |
| Diabetes medication | 2 (6.70) | 8 (20.5) | χ² = 2.62 | 0.105 | 5 (33.3) | 8 (20.5) | χ² = 0.97 | 0.323 |
| n (%) | | | (1) | | | | (1) | |
| Cholesterol medication | 7 (23.3) | 14 (35.9) | χ² = 1.26 | 0.261 | 9 (60.0) | 14 (35.9) | χ² = 2.57 | 0.109 |
| n (%) | | | (1) | | | | (1) | |
| Anticoagulant or | 1 (3.30) | 23 (59.0) | χ² = 23.1 | 0.000 * | 11 (73.3) | 23 (59.0) | χ² = 0.96 | 0.327 |
| antiplatelet n (%) | | | (1) | | | | (1) | |
| rtPA n (%) | 0 (0.00) | 13 (33.3) | χ² = 12.3 | 0.000 * | 0 (0.00) | 13 (33.3) | χ² = 6.59 | 0.011 * |
| | | | (1) | | | | (1) | |

The overall pattern of differential expression between AIS patients and asymptomatic controls observed across the top 10 transcripts in the discovery cohort was also seen when comparing AIS patients and asymptomatic controls in the validation cohort (FIG. 25A). The strong ability of the top 10 transcripts to differentiate between stroke patients and asymptomatic controls in the discovery cohort using kNN was also recapitulated in the validation cohort; the top 10 transcripts used in combination were able to correctly identify 95.6% of subjects with a sensitivity of 92.3% and a specificity of 100% (FIG. 25B).

When comparing AIS patients to stroke mimics, the overall pattern of differential expression observed across the top 10 markers was identical to that observed when comparing AIS patients to asymptomatic controls, however, the magnitude of the difference in expression level was smaller in the case of several transcripts (FIG. 25C). Despite this reduction in the magnitude of differential expression, the top 10 markers used in combination were still able to identify a high percentage of subjects correctly using kNN when comparing AIS patients and stroke mimics, correctly identifying 96.3% of subjects with a specificity of 97.4% and a sensitivity of 93.3% (FIG. 25D).

### Example 12.—Predicting Ischemic Stroke in a Subject

Peripheral blood will be drawn from a subject and collected via PAXgene RNA tubes (Qiagen, Valencia, Calif.). Total RNA will be extracted via PreAnalytiX PAXgene blood RNA kit (Qiagen) and automated using the QIAcube system (Qiagen). The cDNA will be generated from purified RNA using the Applied Biosystems high capacity reverse transcription kit.

Expression levels of biomarkers described herein, such as for example ANTXR2, STK3, PDK4, CD163, MAL, GRAP, ID3, CTSZ, KIF1B, and PLXDC2 in the blood sample will be determined by qPCR. B2M, PPIB, and ACTB gene expression will be use as internal controls. The expression levels of the biomarkers will be compared to a reference. The reference can have average values of the expression levels of the biomarkers evaluated in one or more healthy subject who do not have a risk of stroke.

Ischemic stroke will be predicted when the expression the evaluated biomarkers for example ANTXR2, STK3, PDK4, CD163, CTSZ, KIF1B, and PLXDC2 are increased by at least 1 fold, and the expression of MAL, GRAP, and ID3 are decreased by at least 1 fold in the subject, compared to the reference. The prediction can have a specificity of greater than 98% and a sensitivity of greater than 98%.

### Example 13.—Predicting the Response of an Ischemic Stroke Patient to tPA Treatment

Biomarkers whose expression levels alter in response to tPA treatment will be identified using, for example, the GA/kNN method described in Example 11. Reference ranges of expression levels of the biomarkers that correlate to levels of response to tPA treatment will be established, so that when expression levels of the biomarkers in a patient fall into a reference range. It will be predicted that the patient's response to tPA treatment is at the level correlated with the reference range.

Peripheral blood will be drawn from the patient and collected via PAXgene RNA tubes (Qiagen, Valencia, Calif.). Total RNA will be extracted via PreAnalytiX PAXgene blood RNA kit (Qiagen) and automated using the QIAcube system (Qiagen). The cDNA will be generated from purified RNA using the Applied Biosystems high capacity reverse transcription kit.

Expression levels of biomarkers identified above in the blood sample will be determined by qPCR. B2M, PPIB, and ACTB gene expression will be used as internal controls. The expression levels of the biomarkers will be compared to the reference ranges established above. Based on the range in which the expression levels of the biomarkers fall, the patient's response to tPA treatment will be predicted.

### Example 14.—Identifying Stroke Severity in an Ischemic Stroke Patient

Biomarkers whose expression levels correlate with a stroke severity scale will be identified using, for example, the GA/kNN method described in Example 11. Reference ranges of expression levels of the biomarkers for different levels of severity will be established, so that when expression levels of the biomarkers in a patient fall into a range indicative of a severity level, the stroke severity in the patient is identified.

Peripheral blood will be drawn from the patient and collected via PAXgene RNA tubes (Qiagen, Valencia, Calif.). Total RNA will be extracted via PreAnalytiX PAXgene blood RNA kit (Qiagen) and automated using the QIAcube system (Qiagen). The cDNA will be generated from purified RNA using the Applied Biosystems high capacity reverse transcription kit.

Expression levels of biomarkers identified above in the blood sample will be determined by qPCR. B2M, PPIB, and ACTB gene expression is used as internal controls. The expression levels of the biomarkers will be compared to the reference ranges. Based on the range in which the expression levels of the biomarkers fall, the stroke severity in the patient will be identified. Stroke severity will be (1) no stroke symptoms, (2) minor stroke, (3) moderate stroke, (4) moderate to severe stroke, or (5) severe stroke.

### Example 15.—Cell-Free DNA was Elevated in the Peripheral Circulation of Acute Ischemic Stroke Patients and was Associated with Innate Immune System Activation

Forty-three AIS patients and twenty stroke mimics were recruited. Peripheral blood was sampled at emergency department admission, and plasma cfDNA levels were assessed with qRT-PCR. Peripheral blood neutrophil count was used as a measure of peripheral blood innate immune system status, and infarct volume and NIHSS were used to assess injury severity. cfDNA levels were compared between AIS patients and stroke mimics, and the relationships between cfDNA levels, injury severity, and neutrophil count were assessed.

Samples were collected at ED admission, and within 24 hours of symptom onset, as determined by the time the patient was last known to be free of stroke symptoms. Injury severity was determined according to the NIH stroke scale (NIHSS) at the time of blood draw. Demographic information was collected from either the subject or significant other by a trained clinician.

Venous blood was collected via K2 EDTA vacutainer. For plasma isolation, EDTA-treated blood was spun at 2,000 g for 10 minutes to sediment blood cells. Plasma was collected and spun at 10,000*g for 10 minutes to remove any residual blood cells or debris. Samples were stored at −80° C. until analysis. To identify hemolyzed samples, plasma absorbance was measured at 385 and 414 nm via spectrophotometry and used to calculate a hemolysis score (HS). Non-hemolyzed plasma spiked with serial dilutions of sonicated red blood cells were used as a positive control. Plasma samples with a HS of greater than 0.57 were excluded from cfDNA analysis.

Total DNA was extracted from 200 μL of plasma using the QIAamp DNA micro kit (Qiagen, Valencia, Calif.) and automated using the QIAcube system (Qiagen). Purified DNA was eluted in a 35 μL volume of ultrapure H2O.

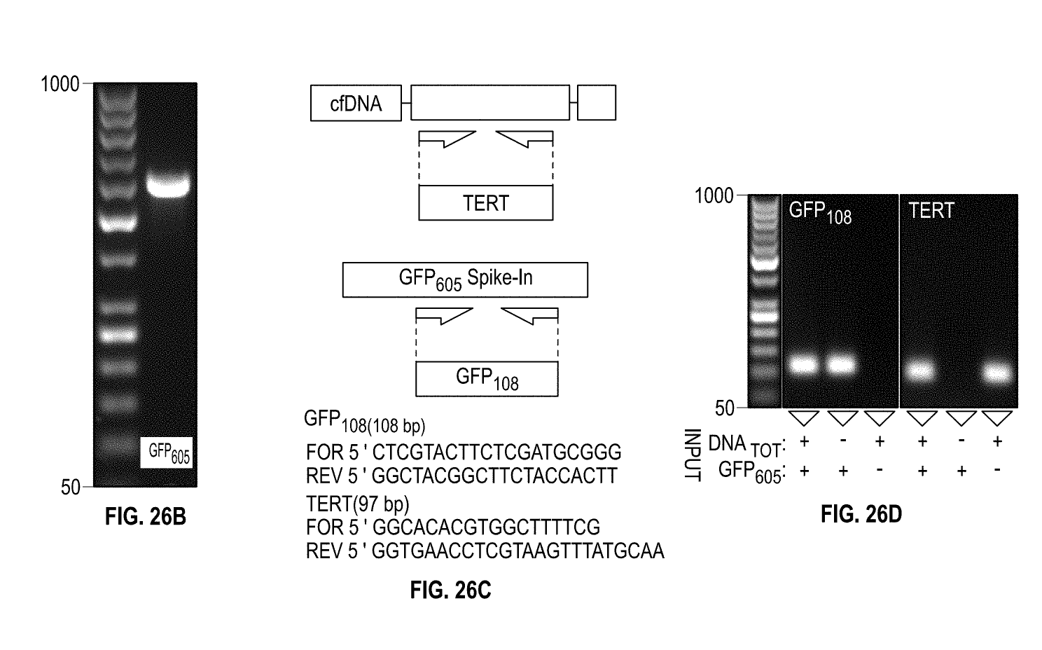

To control for inter-sample variability in DNA extraction efficiency, plasma samples were spiked with a non-human 605 bp DNA fragment originating from the GFP-encoding portion of the pontellina plumata genome (GFP605) prior to DNA extraction. This GFP605 spike-in control was generated via PCR using sequence specific primers and purified pGFP-V-RS plasmid (Origene) as template (FIG. 26A). GFP605 PCR product was electrophoresed via agarose gel and purified using the QIAquick gel extraction kit (Qiagen, FIG. 26B). The concentration and purity of GFP605 was determined via spectophometry. Plasma samples were spiked with purified GFP605 at a final concentration of 10,000 copies per mL. cfDNA levels in plasma eluent were quantified by detection of the single-copy nuclear human Telomerase Reverse Transcriptase (TERT) gene via qPCR. TERT was detected by amplification of a 97 bp fragment. GFP605 spike-in was detected in parallel via amplification of a 108 bp internal fragment (GFP108), which was used for normalization (FIG. 26C). Target sequences were amplified from 5 μL of eluent and detected via SYBR green (PowerSYBR, Thermo-Fisher) on the RotorGeneQ (Qiagen). Raw amplification plots were background corrected and CT values were generated via the RotorGeneQ software package. TERT CT values were normalized via GFP108 CT values, and TERT levels were compared between groups using the 2^{-ΔΔCT} method. All reactions were performed in triplicate and the presence of a single PCR product was confirmed with melting curve analysis. Experiments confirmed that the presence of the GFP605 spike-in did not interfere with TERT detection, and that the GFP spike-in was detectable in the presence of total human DNA (FIG. 26D).

Neuroradiological imaging was performed using either Mill or CT within 24 hours of symptom onset. The Brainlab iPlan software package was used to calculate infarct volume via manual tracing, and all infarct volume calculations were verified by a neuroradiologist.

Neutrophil count was assessed using a standard clinical automated hematology system.

All statistics were performed using the GraphPad Prism statistical software package. Chi-squared analysis was used for inter-group comparison of dichotomous variables, and student t-test was used for inter-group comparison of continuous variables. Spearman's rho was used to test the strength of observed correlations. ROC analysis was used to test the performance of binary classifiers. Optimal cutoff value was determined by the cutoff which yielded the greatest level of combined sensitivity and specificity, and 95% confidence intervals (0.95 CI) were calculated. The level of significance was established at 0.05 for all statistical testing.

### Results:

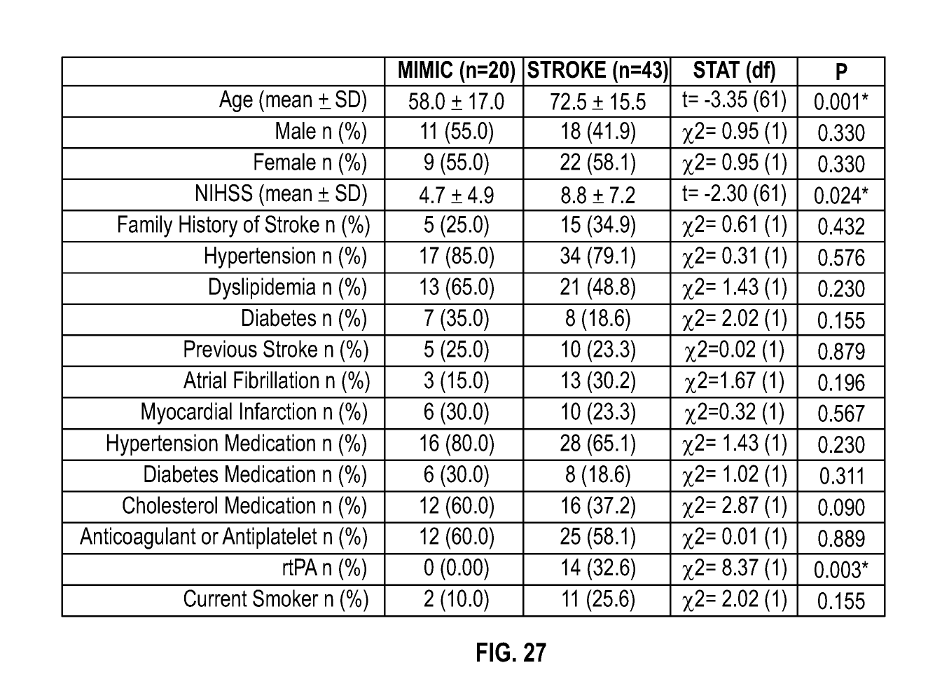

AIS patients were older than stroke mimics, however groups were well matched in terms of cardiovascular disease risk factors and comorbidities (FIG. 27). The median time from symptom onset to blood draw across all subjects was 6.7 hours.

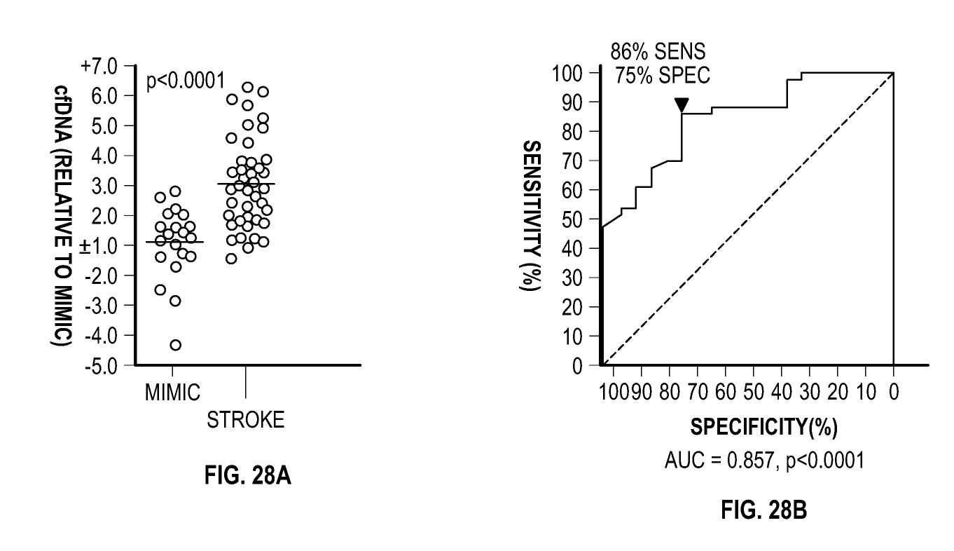

AIS patients displayed close to three-fold higher circulating levels of cfDNA than stroke mimics, as measured by qPCR targeting TERT (FIG. 28A). ROC analysis to test the ability of cfDNA levels to discriminate between AIS patients and stroke mimics produced an area under curve of 0.86, suggesting that cfDNA levels may be diagnostically useful. At optimal cutoff, cfDNA levels were 86% (0.95 CI=72-95%) sensitive and 75% (0.95 CI=51-91%) specific for AIS (FIG. 28B).

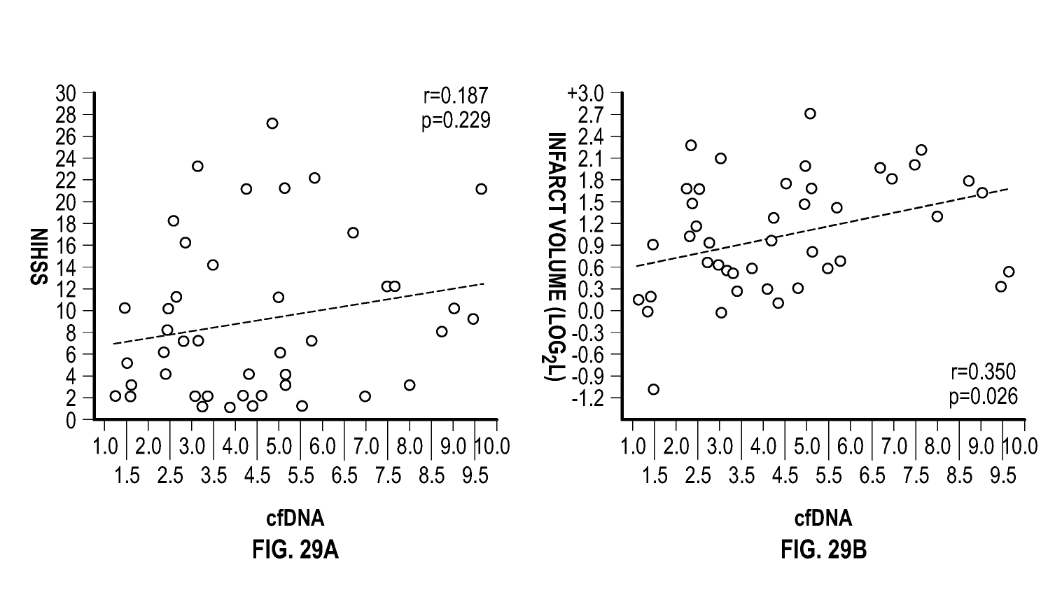

Not only were circulating cfDNA levels higher in AIS patients, they were also positively correlated with injury severity. Circulating cfDNA levels exhibited a weak positive correlation with NUBS (FIG. 29A), however exhibited a significant positive correlation with infarct volume (FIG. 29B).

Circulating cfDNA Levels were Positively Associated with Neutrophil Count:

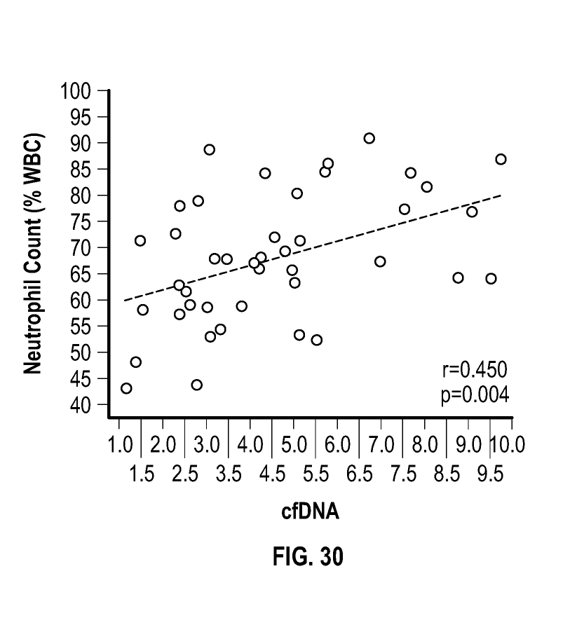

Circulating cfDNA levels were also positively associated with post-stroke neutrophil count in AIS patients, suggesting that cfDNA levels may contribute to post-stroke activation of the innate immune system (FIG. 30).

While some embodiments described herein have been shown and described herein, such embodiments are provided by way of example only. Numerous variations, changes, and substitutions will now occur to those skilled in the art without departing from the disclosure provided herein. It should be understood that various alternatives to the embodiments described herein can be employed in practicing the methods described herein.

1.-135. (canceled)

136. A method comprising:
a) measuring a subject level of peripherally circulating extracellular DNA in a sample from a subject;
b) comparing the subject level to a reference level of peripherally circulating extracellular DNA, wherein the reference level is indicative of a level of peripherally circulating extracellular DNA in a reference sample, wherein the reference sample is a stroke mimic sample; and
c) determining whether the sample or the reference sample has a higher level of peripherally circulating extracellular DNA.

137. The method of claim 136, wherein the subject level is higher than the reference level.

138. The method of claim 137, wherein the subject is an ischemic stroke subject.

139. The method of claim 138, further comprising determining a time of ischemic stroke symptom onset based on the subject level.

140. The method of claim 137, further comprising administering a treatment to the subject.

141. The method of claim 136, further comprising differentiating ischemic stroke from a stroke mimic based on the determining, wherein the subject is an ischemic stroke subject when the subject level is higher than the reference level.

142. The method of claim 141, wherein the differentiating is performed with a sensitivity of at least 80% and a specificity of at least 75%.

143. The method of claim 142, wherein the sensitivity is at least 85%.

144. The method of claim 142, wherein the specificity is at least 80%.

145. The method of claim 136, wherein the peripherally circulating extracellular DNA in the sample comprises an epigenetic marker.

146. The method of claim 145, wherein the epigenetic marker is specific to one or more types of cells.

147. The method of claim 146, wherein the epigenetic marker is specific to a cell from a neurovascular unit.

148. The method of claim 146, wherein the epigenetic marker comprises acetylation, methylation, ubiquitylation, phosphorylation, sumoylation, ribosylation, citrullination, or any combination thereof.

149. The method of claim 136, wherein the sample comprises blood or a fraction thereof.

150. The method of claim 136, wherein stroke severity, activation of innate immune system of the subject or stroke-induced injury is positively correlated with the subject level.

151. The method of claim 136, further comprising measuring a profile of blood cells in the sample from the subject.

152. The method of claim 151, wherein the profile of blood cells comprises white blood cell differentiation, levels of muscle-type creatine kinase and brain-type creatine kinase, a hematocrit percent, a prothrombin time, a white blood cell count, a lymphocyte count, a platelet count, a neutrophil percent in the sample, or a combination thereof.

153. The method of claim 136, wherein the measuring comprises determining a level of a gene or a fragment thereof in the peripherally circulating extracellular DNA in the sample.

154. The method of claim 153, wherein the level of the gene or the fragment thereof were determined by quantitative polymerase chain reaction.

155. The method of claim 153, wherein the gene encodes telomerase reverse transcriptase, beta-globin, cluster of differentiation 240D, a member of albumin family, ribonuclease P RNA component H1, Alu J element, endogenous retrovirus group 3, glyceraldehyde 3-phosphate dehydrogenase, N-acetylglucosamine kinase, or alcohol dehydrogenase.


SEQUENCE LISTING (32 sequences)

SEQ ID NO: 1 - DNA, 22 bp, Artificial Sequence
  Description of Artificial Sequence Synthetic primer
gatctctacttcgtcctggaca

SEQ ID NO: 2 - DNA, 21 bp, Artificial Sequence
  Description of Artificial Sequence Synthetic primer
aaatctctccgcaagttgctg

SEQ ID NO: 3 - DNA, 21 bp, Artificial Sequence
  Description of Artificial Sequence Synthetic primer
cgatgttggaatccgacttgg

SEQ ID NO: 4 - DNA, 23 bp, Artificial Sequence
  Description of Artificial Sequence Synthetic primer
gtctttgtacttgtggtgaggtt

SEQ ID NO: 5 - DNA, 23 bp, Artificial Sequence
  Description of Artificial Sequence Synthetic primer
gacccagtcaccaatcaaaatct

SEQ ID NO: 6 - DNA, 22 bp, Artificial Sequence
  Description of Artificial Sequence Synthetic primer
ggttcatcagcatccgagtaga

SEQ ID NO: 7 - DNA, 21 bp, Artificial Sequence
  Description of Artificial Sequence Synthetic primer
gcgggagagtggaagtgaaag

SEQ ID NO: 8 - DNA, 22 bp, Artificial Sequence
  Description of Artificial Sequence Synthetic primer
gttacaaatcacagagaccgct

SEQ ID NO: 9 - DNA, 19 bp, Artificial Sequence
  Description of Artificial Sequence Synthetic primer
gccctcttttacctcagcg

SEQ ID NO: 10 - DNA, 23 bp, Artificial Sequence
  Description of Artificial Sequence Synthetic primer
gcaatgttttcatggtagtgcct

SEQ ID NO: 11 - DNA, 19 bp, Artificial Sequence
  Description of Artificial Sequence Synthetic primer
agcccttgctcaagtcacc

SEQ ID NO: 12 - DNA, 21 bp, Artificial Sequence
  Description of Artificial Sequence Synthetic primer
cgtaactccgtgggaagaagc

SEQ ID NO: 13 - DNA, 20 bp, Artificial Sequence
  Description of Artificial Sequence Synthetic primer
gagaggcactcagcttagcc

SEQ ID NO: 14 - DNA, 22 bp, Artificial Sequence
  Description of Artificial Sequence Synthetic primer
tccttttgtcgttggagatgac

SEQ ID NO: 15 - DNA, 19 bp, Artificial Sequence
  Description of Artificial Sequence Synthetic primer
cagcggatctgcccaagag

SEQ ID NO: 16 - DNA, 19 bp, Artificial Sequence
  Description of Artificial Sequence Synthetic primer
cgatgacgttctgcacgga

SEQ ID NO: 17 - DNA, 23 bp, Artificial Sequence
  Description of Artificial Sequence Synthetic primer
actcagatcgaggaggatacaga

SEQ ID NO: 18 - DNA, 20 bp, Artificial Sequence
  Description of Artificial Sequence Synthetic primer
ccggctggcagaatcagatg

SEQ ID NO: 19 - DNA, 23 bp, Artificial Sequence
  Description of Artificial Sequence Synthetic primer
aaacaagggtaatttgcgtgtgc

SEQ ID NO: 20 - DNA, 22 bp, Artificial Sequence
  Description of Artificial Sequence Synthetic primer
gtaactgccaacttggacagat

SEQ ID NO: 21 - DNA, 23 bp, Artificial Sequence
  Description of Artificial Sequence Synthetic primer
aagtcaccgtcaaggtgtatttt

SEQ ID NO: 22 - DNA, 23 bp, Artificial Sequence
  Description of Artificial Sequence Synthetic primer
tgctgtttttgtagccaaatcct

SEQ ID NO: 23 - DNA, 21 bp, Artificial Sequence
  Description of Artificial Sequence Synthetic primer
gaggctatccagcgtactcca

SEQ ID NO: 24 - DNA, 21 bp, Artificial Sequence
  Description of Artificial Sequence Synthetic primer
cggcaggcatactcatctttt

SEQ ID NO: 25 - DNA, 21 bp, Artificial Sequence
  Description of Artificial Sequence Synthetic primer
catgtacgttgctatccaggc

SEQ ID NO: 26 - DNA, 21 bp, Artificial Sequence
  Description of Artificial Sequence Synthetic primer
ctccttaatgtcacgcacgat

SEQ ID NO: 27 - DNA, 20 bp, Artificial Sequence
  Description of Artificial Sequence Synthetic primer
gttgctgtgatcctcctcca

SEQ ID NO: 28 - DNA, 19 bp, Artificial Sequence
  Description of Artificial Sequence Synthetic primer
ccgccatggagatcgagtg

SEQ ID NO: 29 - DNA, 20 bp, Artificial Sequence
  Description of Artificial Sequence Synthetic primer
ctcgtacttctcgatgcggg

SEQ ID NO: 30 - DNA, 20 bp, Artificial Sequence
  Description of Artificial Sequence Synthetic primer
ggctacggcttctaccactt

SEQ ID NO: 31 - DNA, 18 bp, Artificial Sequence
  Description of Artificial Sequence Synthetic primer
ggcacacgtggcttttcg

SEQ ID NO: 32 - DNA, 24 bp, Artificial Sequence
  Description of Artificial Sequence Synthetic primer
ggtgaacctcgtaagtttatgcaa